(19) United States
(12) Patent Application Publication
(10) Pub. No.: US 2026/0241039 A1
(43) Pub. Date: Aug. 20, 2026

(54) FLUORESCENT PROBES FOR MAGL

(71) Applicant: Hoffmann-La Roche Inc., Little Falls, NJ (US)

(72) Inventors: Joerg BENZ, Rheinfelden (DE); Maude GIROUD, Basel (CH); Uwe GRETHER, Efringen-Kirchen (DE); Monica GUBERMAN, Berlin (DE); Axel HENTSCH, Berlin (DE); Bernd KUHN, Reinach BL (CH); Rainer Eugen MARTIN, Basel (CH); Marc NAZARE, Berlin (DE); Fionn Susannah O’HARA, Basel (CH); Jerome PAUL, Berlin (DE); Bernd PUELLMANN, Aesch (CH); Martin RITTER, Mumpf (CH)

(73) Assignee: Hoffmann-La Roche Inc., Little Falls, NJ (US)

(21) Appl. No.: 18/876,839

(22) PCT Filed: Jun. 22, 2023
(86) PCT No.: PCT/EP2023/066902

§ 371 Date: Dec. 19, 2024

(30) Foreign Application Priority Data
Jun. 24, 2022 (EP) 22181018.7

(51) Int. Cl. A61K 49/00; C07D 498/04; C07F 5/02; G01N 33/557; G01N 33/573; G01N 33/58

(52) U.S. Cl. CPC A61K 49/0021; C07D 498/04; C07F 5/022; G01N 33/557; G01N 33/573; G01N 33/582; G01N 2333/918

(57) ABSTRACT

The invention provides compounds having the general formula (I) wherein A, L, R¹ and R² are as described herein, compositions including the compounds, processes of manufacturing the compounds and methods of using the compounds.

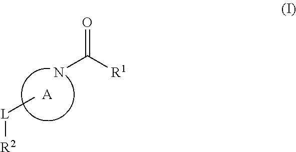

### FIELD OF THE INVENTION

The present invention relates to organic compounds useful as fluorescent probes for monoacylglycerol lipase (MAGL).

### BACKGROUND OF THE INVENTION

Fluorescent imaging probes have emerged as high resolution tools to investigate localization, e.g. expression levels and protein distribution in health and disease, structure, dynamics and function of proteins in living cells (L. A. Stoddart, L. E. Kilpatrick, S. J. Briddon, S. J. Hill, Neuropharmacology 2015, 98, 48-57). Such probes can e.g. be applied in flow cytometry fluorescence-activated cell sorting (FACS) experiments or cellular trafficking studies using confocal live cell imaging. Furthermore, fluorescent imaging probes allow for real-time monitoring of ligand-receptor interactions and protein visualization with high spatiotemporal precision (A. J. Vernall, S. J. Hill, B. Kellam, Br. J Pharmacol. 2014, 171, 1073-1084; C. Iliopoulos-Tsoutsouvas, R. N. Kulkarni, A. Makriyannis, S. P. Nikas, Expert Opin. Drug Discov. 2018, 13, 933-947). In addition, such probes offer the potential for generating equilibrium and kinetic binding data in a high-throughput fashion, without handling radioactive material using e.g. time-resolved fluorescence resonance energy transfer (TR-FRET). Fluorescent imaging probes can also be useful to support the translation of preclinical pharmacological animal data to clinics and can be applied for dose selection in humans. They can e.g. be used as markers of target engagement via the generation of ex vivo quantitative receptor binding data in whole blood. Depending on the respective application, a fluorescent imaging probe needs to match specific criteria, including affinity, selectivity and specificity for the respective target, favorable photophysical properties, and applicability across distinct techniques and cell types.

### SUMMARY OF THE INVENTION

In a first aspect, the present invention provides a compound of Formula (I)

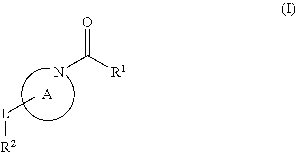

or pharmaceutically acceptable salts thereof, wherein A, L, R¹, and R² are as defined herein.

In a further aspect, the present invention provides a process of manufacturing the compounds of formula (I) as described herein, comprising:
(a) reacting a first amine 1,

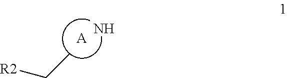

wherein A and R² are as defined herein;
with a second amine selected from:

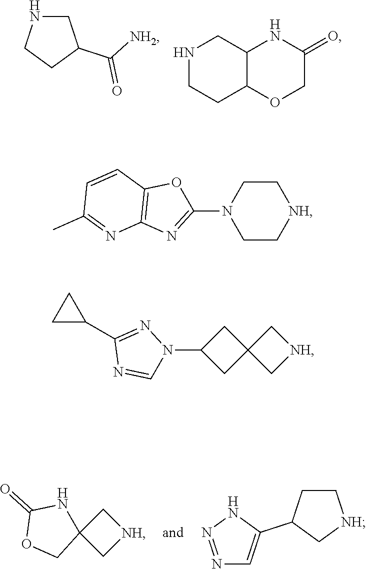 [SMILES: NC(=O)C1CCNC1 ; O=C1COC2CCNCC2N1 ; Cc1ccc2oc(N3CCNCC3)nc2n1 ; c1nc(C2CC2)nn1C1CC2(CNC2)C1 ; O=C1NC2(CNC2)CO1 ; c1nn[nH]c1C1CCNC1]

in the presence of a base and a urea forming reagent,
to form said compound of formula (I); or
(b) reacting an amine 1,

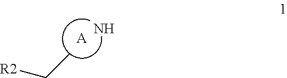

wherein A and R² are as defined herein;
with a carboxylic acid selected from:

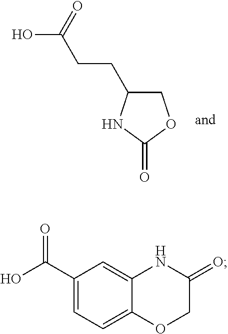 [SMILES: O=C(O)CCC1COC(=O)N1 ; O=C1COc2ccc(C(=O)O)cc2N1]

in the presence of a base and an amide coupling reagent,
to form said compound of formula (I).

In a further aspect, the present invention provides a compound of formula (I) as described herein, when manufactured according to the processes described herein.

In a further aspect, the present invention provides a method of studying monoacylglycerol lipase (MAGL) occupancy, comprising contacting MAGL with a compound of formula (I) described herein.

In a further aspect, the present invention provides a method of diagnostic imaging of monoacylglycerol lipase (MAGL) in a mammal, comprising contacting MAGL with a compound of formula (I) described herein.

In a further aspect, the present invention provides a method of generating monoacylglycerol lipase (MAGL) equilibrium and kinetic binding data, comprising contacting MAGL with a compound of formula (I) described herein.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 12 carbon atoms. In some preferred embodiments, the alkyl group contains 1 to 6 carbon atoms, e.g., 1, 2, 3, 4, 5, or 6 carbon atoms (“C₁-C₆-alkyl”). In other embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. A particularly preferred, yet non-limiting example of alkyl is methyl.

The term “alkoxy” refers to an alkyl group, as previously defined, attached to the parent molecular moiety via an oxygen atom. Unless otherwise specified, the alkoxy group contains 1 to 12 carbon atoms. In some preferred embodiments, the alkoxy group contains 1 to 6 carbon atoms (“C₁-C₆-alkoxy”). In other embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “aryl” refers to a monocyclic, bicyclic, or tricyclic carbocyclic ring system having a total of 6 to 14 ring members (“C₆₋₁₄-aryl”), preferably, 6 to 12 ring members, and more preferably 6 to 10 ring members, and wherein at least one ring in the system is aromatic. Some non-limiting examples of aryl include phenyl and 9H-fluorenyl (e.g. 9H-fluoren-9-yl). A particularly preferred, yet non-limiting example of aryl is phenyl.

The term “heteroaryl” refers to a mono- or multivalent, monocyclic or bicyclic ring system having a total of 5 to 14 ring members, preferably, 5 to 12 ring members, and more preferably 5 to 10 ring members, wherein at least one ring in the system is aromatic, and at least one ring in the system contains one or more heteroatoms. Preferably, “heteroaryl” refers to a 5-10 membered heteroaryl comprising 1, 2, 3 or 4 heteroatoms independently selected from O, S and N. Most preferably, “heteroaryl” refers to a 5-10 membered heteroaryl comprising 1 to 2 heteroatoms independently selected from O, S and N. Some preferred, yet non-limiting examples of heteroaryl include thiazolyl (e.g. thiazol-2-yl); oxazolyl (e.g. oxazol-2-yl); oxadiazolyl; 5,6-dihydro-4H-cyclopenta[d]thiazol-2-yl; 1,2,4-oxadiazol-5-yl; pyridyl (e.g. 2-pyridyl); pyrazolyl (e.g. pyrazol-1-yl); triazolyl; tetrazolyl; pyrazinyl; imidazolyl (e.g. imidazole-1-yl); benzoxazolyl (e.g. benzoxazol-2-yl), 2,3-dihydrobenzofuranyl; and oxazolo[5,4-c]pyridin-2-yl. Some particularly preferred, yet non-limiting examples of heteroaryl include oxadiazolyl; pyridyl; triazolyl; tetrazolyl; pyrazinyl and imidazolyl.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are hydrochloride salts.

The term “protective group” (PG) denotes the group which selectively blocks a reactive site in a multifunctional compound such that a chemical reaction can be carried out selectively at another unprotected reactive site in the meaning conventionally associated with it in synthetic chemistry. Protective groups can be removed at the appropriate point. Exemplary protective groups are amino-protective groups, carboxy-protective groups or hydroxy-protective groups. Particular protective groups are the tert-butoxycarbonyl (Boc), benzyloxycarbonyl (Cbz), fluorenylmethoxycarbonyl (Fmoc) and benzyl (Bn). Further particular protective groups are the tert-butoxycarbonyl (Boc) and the fluorenylmethoxycarbonyl (Fmoc). More particular protective group is the tert-butoxycarbonyl (Boc). Exemplary protective groups and their application in organic synthesis are described, for example, in “Protective Groups in Organic Chemistry” by T. W. Greene and P. G. M. Wutts, 5th Ed., 2014, John Wiley & Sons, N.Y.

The term “urea forming reagent” refers to a chemical compound that is able to render a first amine to a species that will react with a second amine, thereby forming an urea derivative. Non-limiting examples of a urea forming reagent include bis(trichloromethyl) carbonate, phosgene, trichloromethyl chloroformate, (4-nitrophenyl)carbonate and 1,1′-carbonyldiimidazole. The urea forming reagents described in G. Sartori et al., Green Chemistry 2000, 2, 140 are incorporated herein by reference.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The abbreviation “MAGL” refers to the enzyme monoacylglycerol lipase. The terms “MAGL” and “monoacylglycerol lipase” are used herein interchangeably.

### Compounds of the Invention

In a first aspect (A1), the present invention provides a compound of Formula (I)

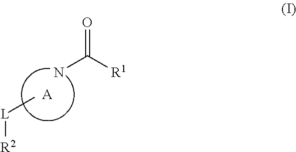

or a pharmaceutically acceptable salt thereof, wherein:
R¹ is selected from:

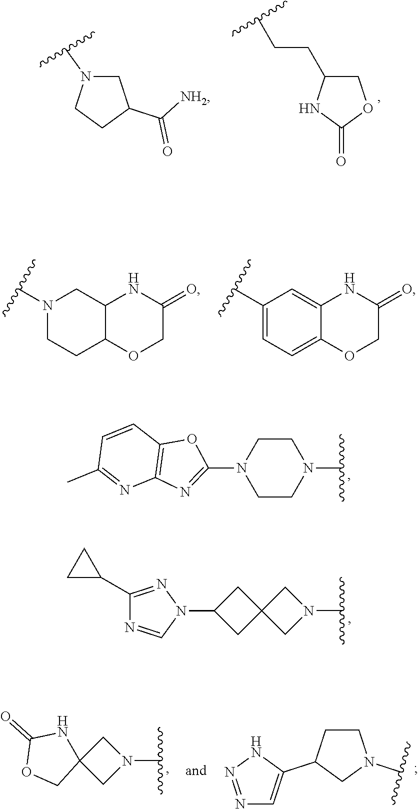

wherein a wavy line indicates the point of attachment to the carbonyl group of formula (I);
R² is selected from:

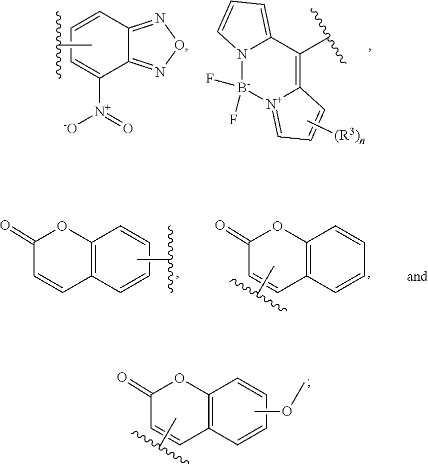

wherein a wavy line indicates the point of attachment to L;
each R³ is independently selected from hydrogen, halogen, C₁-C₆-alkyl, C₁-C₆-alkoxy, phenyl,

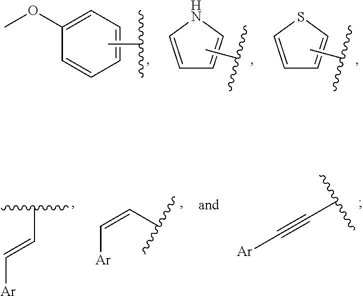

n is 1, 2 or 3;
Ar is C₆-C₁₀-aryl or 5- to 14-membered heteroaryl;
A is selected from:
   (i)

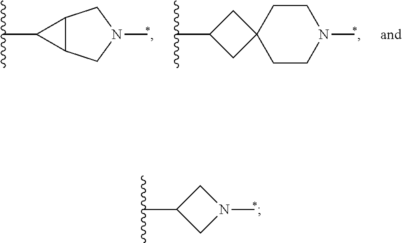

and
L is selected from a covalent bond, —CH₂—, —O—, —OCH₂—, —CH₂O— and —CH₂OCH₂—; and
(ii)

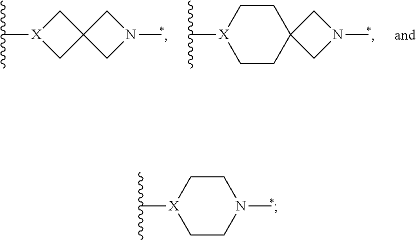

wherein
(i) X is N; and L is selected from a covalent bond, —CH₂—, and —OCH₂—; or
(ii) X is CH; and L is selected from a covalent bond, —CH₂—, —NH—, —NMe-, —O—, —OCH₂—, —CH₂O— and —CH₂OCH₂—; or
(iii) X and L, taken together, form a group

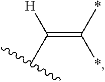

wherein the wavy line indicates the point of attachment of L to R², and the two asterisks indicate the points of attachment of X to the respective neighbouring atoms within ring A;
wherein a wavy line indicates the point of attachment to L; and
an asterisk indicates the point of attachment to the carbonyl group of formula (I).

In one embodiment, the present invention provides a compound of formula (I) as described herein, or a pharmaceutically acceptable salt thereof, wherein R¹ is selected from

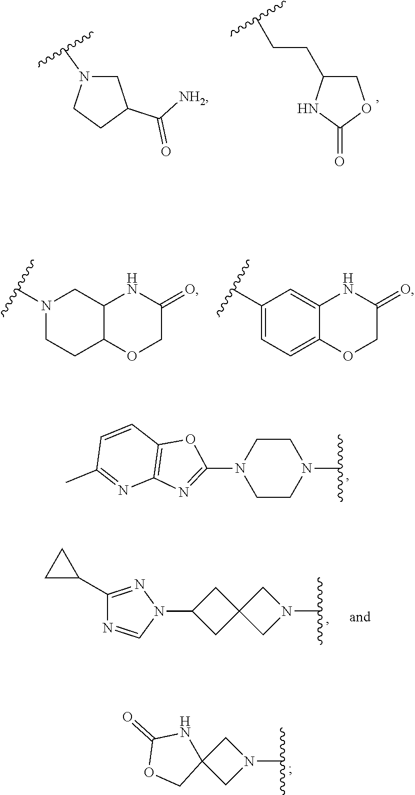

wherein a wavy line indicates the point of attachment to the carbonyl group of formula (I).

In one embodiment, the present invention provides a compound of formula (I) as described herein, or a pharmaceutically acceptable salt thereof, wherein R¹ is selected from

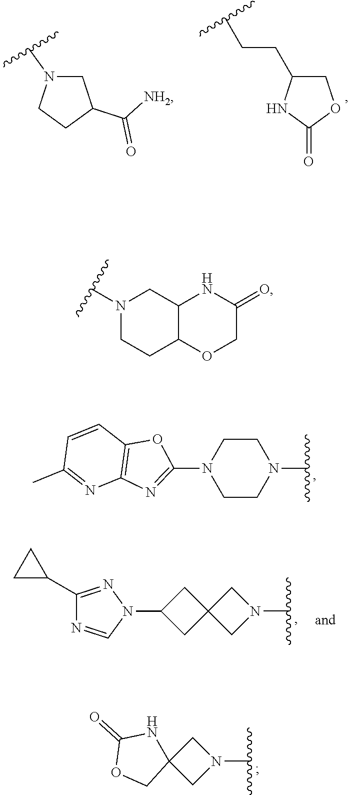 [SMILES: *N1CCC(C(N)=O)C1 ; *CCC1COC(=O)N1 ; *N1CCC2OCC(=O)NC2C1 ; *N1CCN(c2nc3nc(C)ccc3o2)CC1 ; *N1CC2(CC(n3cnc(C4CC4)n3)C2)C1 ; *N1CC2(COC(=O)N2)C1]

wherein a wavy line indicates the point of attachment to the carbonyl group of formula (I).

In a preferred embodiment, the present invention provides a compound of formula (I) as described herein, or a pharmaceutically acceptable salt thereof, wherein R¹ is selected from

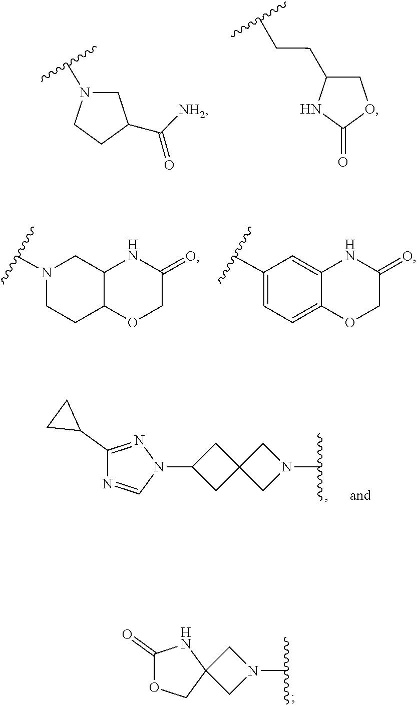

wherein a wavy line indicates the point of attachment to the carbonyl group of formula (I).

In a preferred embodiment, the present invention provides a compound of formula (I) as described herein, or a pharmaceutically acceptable salt thereof, wherein R¹ is selected from

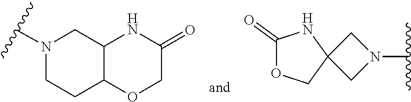 [SMILES: *N1CCC2OCC(=O)NC2C1 ; *N1CC2(COC(=O)N2)C1]

wherein a wavy line indicates the point of attachment to the carbonyl group of formula (I).

In a particularly preferred embodiment, the present invention provides a compound of formula (I) as described herein, or a pharmaceutically acceptable salt thereof, wherein R¹ is selected from

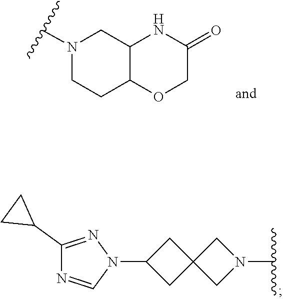 [SMILES: *N1CCC2OCC(=O)NC2C1 ; *N1CC2(CC(n3cnc(C4CC4)n3)C2)C1]

wherein a wavy line indicates the point of attachment to the carbonyl group of formula (I).

In a particularly preferred embodiment, the present invention provides a compound of formula (I) as described herein, or a pharmaceutically acceptable salt thereof, wherein R¹ is

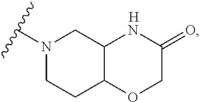

wherein a wavy line indicates the point of attachment to the carbonyl group of formula (I).

In a particularly preferred embodiment, the present invention provides a compound of formula (I) as described herein, or a pharmaceutically acceptable salt thereof, wherein R¹ is

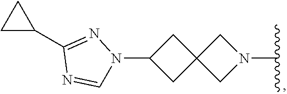 [SMILES: *N1CC2(CC(n3cnc(C4CC4)n3)C2)C1]

wherein a wavy line indicates the point of attachment to the carbonyl group of formula (I).

In a particularly preferred embodiment, the present invention provides a compound of formula (I) as described herein, or a pharmaceutically acceptable salt thereof, wherein R¹ is

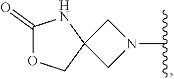 [SMILES: *N1CC2(COC(=O)N2)C1]

wherein a wavy line indicates the point of attachment to the carbonyl group of formula (I).

In one embodiment, the present invention provides a compound of formula (I) as described herein, or a pharmaceutically acceptable salt thereof, wherein:
R² is selected from:

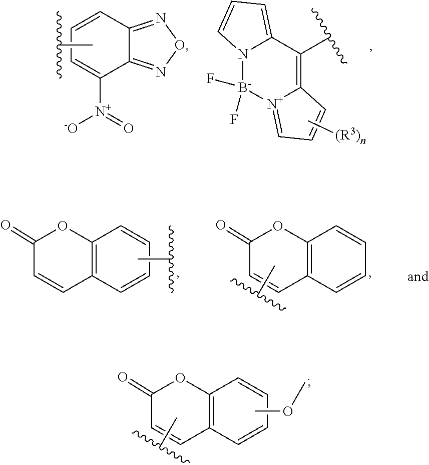

wherein a wavy line indicates the point of attachment to L;
R³ is selected from hydrogen, phenyl, and

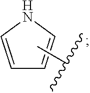

and
n is 1.

In a preferred embodiment, the present invention provides a compound of formula (I) as described herein, or a pharmaceutically acceptable salt thereof, wherein:
R² is selected from:

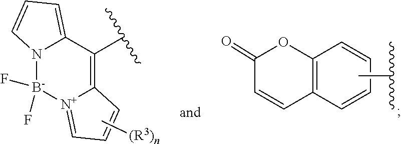

wherein a wavy line indicates the point of attachment to L;
R³ is selected from hydrogen and

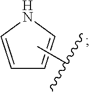

and
n is 1.

In a particularly preferred embodiment, the present invention provides a compound of formula (I) as described herein, or a pharmaceutically acceptable salt thereof, wherein:
R² is:

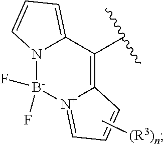

wherein a wavy line indicates the point of attachment to L;
R³ is hydrogen; and
n is 1.

In one embodiment, the present invention provides a compound of formula (I) as described herein, or a pharmaceutically acceptable salt thereof, wherein:
A is selected from:
   (i)

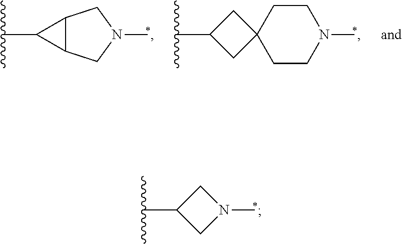

and
L is selected from a covalent bond, —O—, and —OCH₂—; and
(ii)

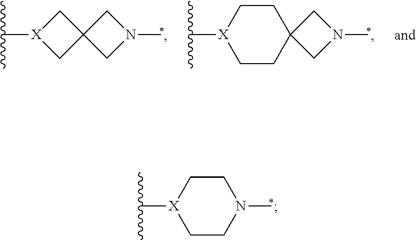

wherein
(i) X is N; and L is —CH₂—; or
(ii) X is CH; and L is selected from —CH₂—, —NH— and —O—; or
(iii) X and L, taken together, form a group

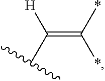

wherein the wavy line indicates the point of attachment of L to R², and the two is asterisks indicate the points of attachment of X to the respective neighbouring atoms within ring A;
wherein a wavy line indicates the point of attachment to L; and
an asterisk indicates the point of attachment to the carbonyl group of formula (I).

In a preferred embodiment, the present invention provides a compound of formula (I) as described herein, or a pharmaceutically acceptable salt thereof, wherein:
A is selected from:
(i)

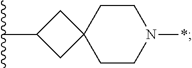

and
L is —O—; and
(ii)

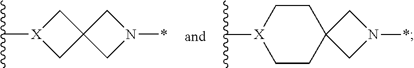

wherein
(i) X is CH; and L is selected from —CH₂— and —O—; or
(ii) X and L, taken together, form a group

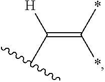

wherein the wavy line indicates the point of attachment of L to R², and the two asterisks indicate the points of attachment of X to the respective neighbouring atoms within ring A;
wherein a wavy line indicates the point of attachment to L; and
an asterisk indicates the point of attachment to the carbonyl group of formula (I).

In a particularly preferred embodiment, the present invention provides a compound of formula (I) as described herein, or a pharmaceutically acceptable salt thereof, wherein:
A is

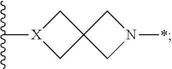

X is CH; ana
L is —CH₂—;
wherein the wavy line indicates the point of attachment to L; and
the asterisk indicates the point of attachment to the carbonyl group of formula (I).

In one embodiment, the present invention provides a compound of formula (I) as described herein, or a pharmaceutically acceptable salt thereof, wherein:
R¹ is selected from

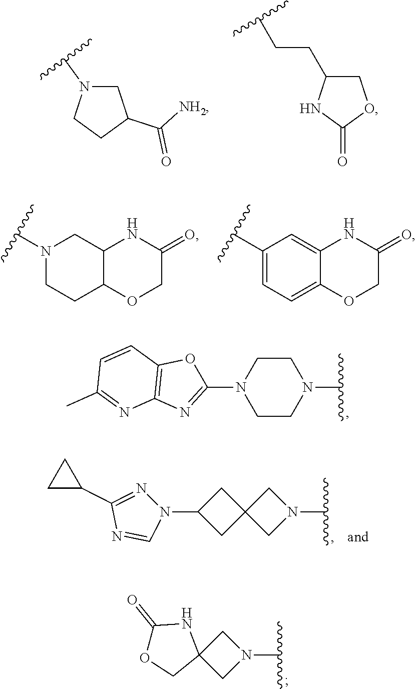

wherein a wavy line indicates the point of attachment to the carbonyl group of formula (I);
R² is selected from:

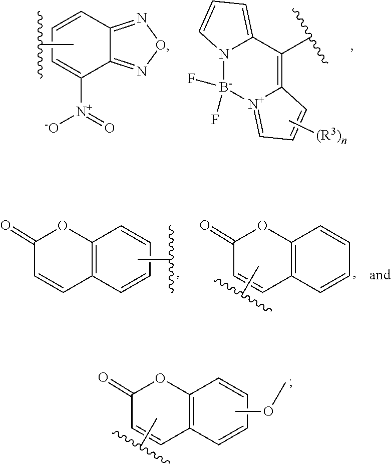

wherein a wavy line indicates the point of attachment to L;
R³ is selected from hydrogen, phenyl, and

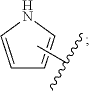

n is 1;
A is selected from:
   (i)

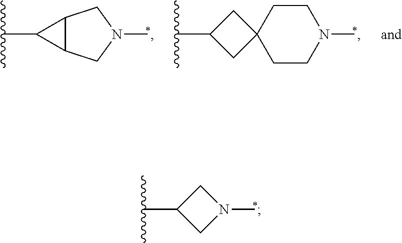

and
L is selected from a covalent bond, —O—, and —OCH₂—; and
(ii)

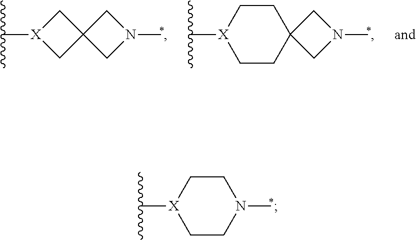

wherein
(i) X is N; and L is —CH₂—; or
(ii) X is CH; and L is selected from —CH₂—, —NH— and —O—; or
(iii) X and L, taken together, form a group

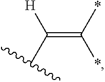

wherein the wavy line indicates the point of attachment of L to R², and the two asterisks indicate the points of attachment of X to the respective neighbouring atoms within ring A;
wherein a wavy line indicates the point of attachment to L; and
an asterisk indicates the point of attachment to the carbonyl group of formula (I).

In a preferred embodiment, the present invention provides a compound of formula (I) as described herein, or a pharmaceutically acceptable salt thereof, wherein:
R¹ is selected from

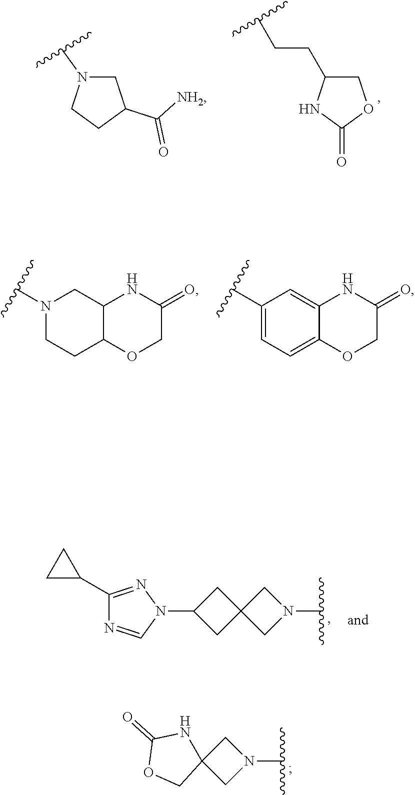

wherein a wavy line indicates the point of attachment to the carbonyl group of formula (I);
R² is selected from:

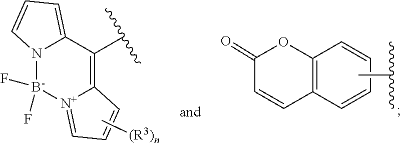

wherein a wavy line indicates the point of attachment to L;
R³ is selected from hydrogen and

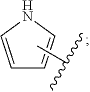

n is 1;
A is selected from:
   (i)

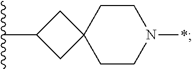

and
L is —O—; and
(ii)

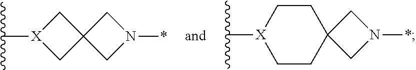

wherein
(i) X is CH; and L is selected from —CH₂— and —O—; or
(ii) X and L, taken together, form a group

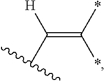

wherein the wavy line indicates the point of attachment of L to R², and the two asterisks indicate the points of attachment of X to the respective neighbouring atoms within ring A;
wherein a wavy line indicates the point of attachment to L; and
an asterisk indicates the point of attachment to the carbonyl group of formula (I).

In a particularly preferred embodiment, the present invention provides a compound of formula (I) as described herein, or a pharmaceutically acceptable salt thereof, wherein:
R¹ is selected from

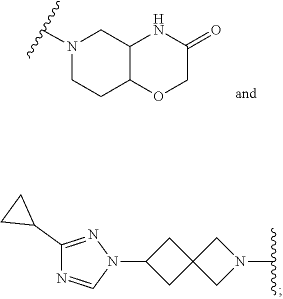 [SMILES: *N1CCC2OCC(=O)NC2C1 ; *N1CC2(CC(n3cnc(C4CC4)n3)C2)C1]

wherein a wavy line indicates the point of attachment to the carbonyl group of formula (I);
R² is:

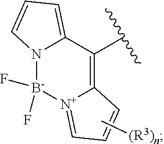

wherein a wavy line indicates the point of attachment to L;
R³ is hydrogen;
n is 1;
A is

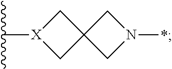

X is CH; and
L is —CH₂—;
   wherein the wavy line indicates the point of attachment to L; and the asterisk indicates the point of attachment to the carbonyl group of formula (I).

In one embodiment, the present invention provides a compound of formula (I) as described herein, or a pharmaceutically acceptable salt thereof, wherein:
R¹ is selected from

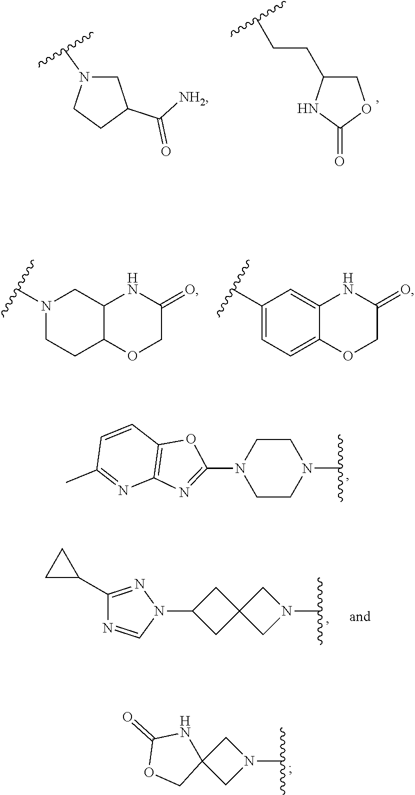

wherein a wavy line indicates the point of attachment to the carbonyl group of formula (I).

In one embodiment, the present invention provides a compound of formula (I) as described herein, or a pharmaceutically acceptable salt thereof, wherein R² is selected from:

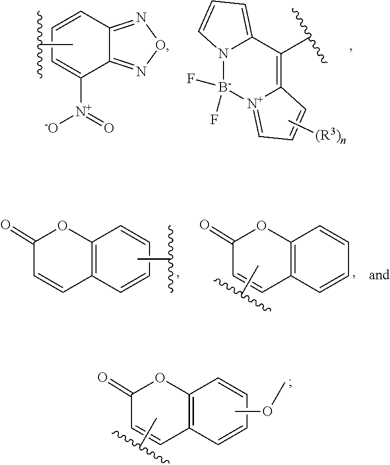

wherein a wavy line indicates the point of attachment to L.

In one embodiment, the present invention provides a compound of formula (I) as described herein, or a pharmaceutically acceptable salt thereof, wherein R³ is selected from hydrogen, phenyl, and

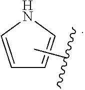

In one embodiment, the present invention provides a compound of formula (I) as described herein, or a pharmaceutically acceptable salt thereof, wherein n is 1.

In one embodiment, the present invention provides a compound of formula (I) as described herein, or a pharmaceutically acceptable salt thereof, wherein A is selected from:
(i)

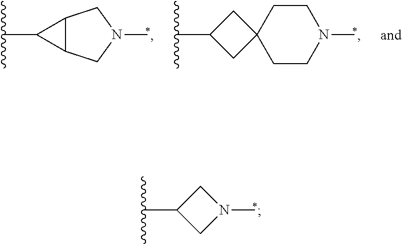

and
L is selected from a covalent bond, —O—, and —OCH₂—; and
(ii)

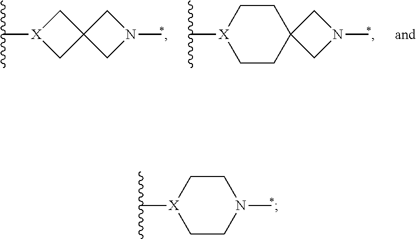

wherein
(i) X is N; and L is —CH₂—; or
(ii) X is CH; and L is selected from —CH₂—, —NH— and —O—; or
(iii) X and L, taken together, form a group

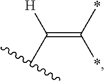

wherein the wavy line indicates the point of attachment of L to R², and the two asterisks indicate the points of attachment of X to the respective neighbouring atoms within ring A;
wherein a wavy line indicates the point of attachment to L; and
an asterisk indicates the point of attachment to the carbonyl group of formula (I).

In a preferred embodiment, the present invention provides a compound of formula (I) as described herein, or a pharmaceutically acceptable salt thereof, wherein R¹ is selected from

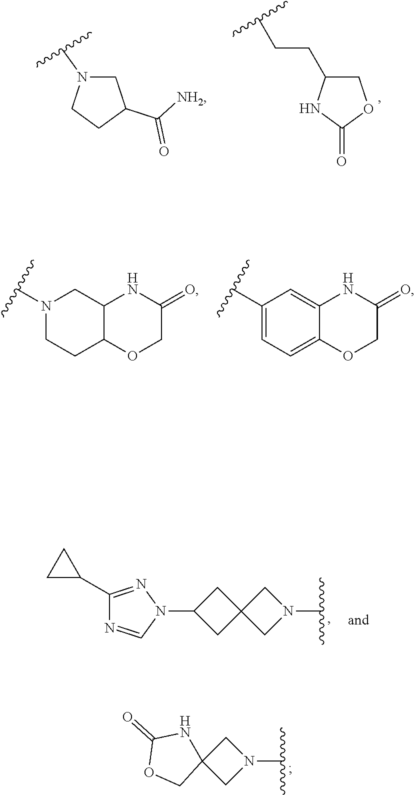

wherein a wavy line indicates the point of attachment to the carbonyl group of formula (I).

In a preferred embodiment, the present invention provides a compound of formula (I) as described herein, or a pharmaceutically acceptable salt thereof, wherein R² is selected from:

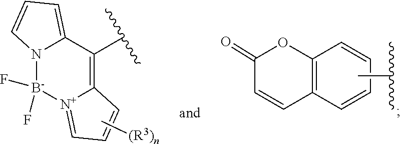

wherein a wavy line indicates the point of attachment to L.

In a preferred embodiment, the present invention provides a compound of formula (I) as described herein, or a pharmaceutically acceptable salt thereof, wherein R³ is selected from hydrogen and

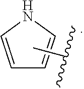

In a preferred embodiment, the present invention provides a compound of formula (I) as described herein, or a pharmaceutically acceptable salt thereof, wherein A is selected from:
(i)

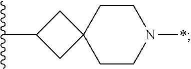

and
L is —O—; and
(ii)

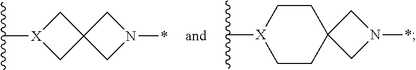

wherein
(i) X is CH; and L is selected from —CH₂— and —O—; or
(ii) X and L, taken together, form a group

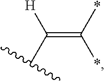

wherein the wavy line indicates the point of attachment of L to R², and the two asterisks indicate the points of attachment of X to the respective neighbouring atoms within ring A;
wherein a wavy line indicates the point of attachment to L; and
an asterisk indicates the point of attachment to the carbonyl group of formula (I).

In a particularly preferred embodiment, the present invention provides a compound of formula (I) as described herein, or a pharmaceutically acceptable salt thereof, wherein R¹ is selected from

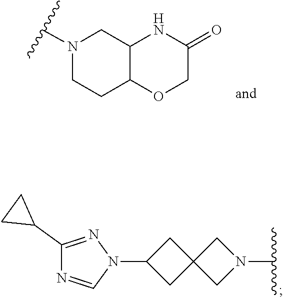 [SMILES: *N1CCC2OCC(=O)NC2C1 ; *N1CC2(CC(n3cnc(C4CC4)n3)C2)C1]

wherein a wavy line indicates the point of attachment to the carbonyl group of formula (I).

In a particularly preferred embodiment, the present invention provides a compound of formula (I) as described herein, or a pharmaceutically acceptable salt thereof, wherein R¹ is

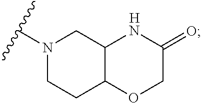

wherein a wavy line indicates the point of attachment to the carbonyl group of formula (I).

In a particularly preferred embodiment, the present invention provides a compound of formula (I) as described herein, or a pharmaceutically acceptable salt thereof, wherein R¹ is

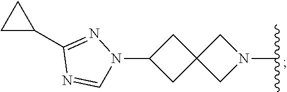 [SMILES: *N1CC2(CC(n3cnc(C4CC4)n3)C2)C1]

wherein a wavy line indicates the point of attachment to the carbonyl group of formula (I).

In a particularly preferred embodiment, the present invention provides a compound of formula (I) as described herein, or a pharmaceutically acceptable salt thereof, wherein R² is

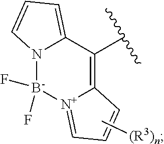

wherein a wavy line indicates the point of attachment to L.

In a particularly preferred embodiment, the present invention provides a compound of formula (I) as described herein, or a pharmaceutically acceptable salt thereof, wherein R² is

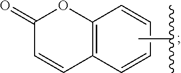

wherein a wavy line indicates the point of attachment to L.

In a particularly preferred embodiment, the present invention provides a compound of formula (I) as described herein, or a pharmaceutically acceptable salt thereof, wherein R³ is hydrogen.

In a particularly preferred embodiment, the present invention provides a compound of formula (I) as described herein, or a pharmaceutically acceptable salt thereof, wherein R³ is

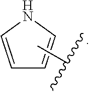

In a particularly preferred embodiment, the present invention provides a compound of formula (I) as described herein, or a pharmaceutically acceptable salt thereof, wherein:
A is

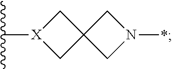

X is CH; and
L is —CH₂—;
   wherein the wavy line indicates the point of attachment to L; and
   the asterisk indicates the point of attachment to the carbonyl group of formula (I).

In a particularly preferred embodiment, the present invention provides a compound of formula (I) as described herein, or a pharmaceutically acceptable salt thereof, wherein:
A is

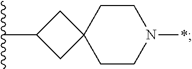

and
L is —O—.

In a particularly preferred embodiment, the present invention provides a compound of formula (I) as described herein, or a pharmaceutically acceptable salt thereof, wherein:
A is

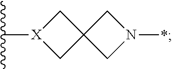

X is CH; and
L is selected from —CH₂— and —O—;
   wherein a wavy line indicates the point of attachment to L; and
   an asterisk indicates the point of attachment to the carbonyl group of formula (I).

In a particularly preferred embodiment, the present invention provides a compound of formula (I) as described herein, or a pharmaceutically acceptable salt thereof, wherein:
A is

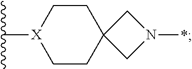

X is CH; and
L is selected from —CH₂— and —O—; and
   wherein a wavy line indicates the point of attachment to L; and
   an asterisk indicates the point of attachment to the carbonyl group of formula (I).

In a particularly preferred embodiment, the present invention provides a compound of formula (I) as described herein, or a pharmaceutically acceptable salt thereof, wherein:
A is

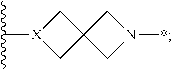

X and L, taken together, form a group

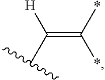

wherein the wavy line indicates the point of attachment of L to R², and the two asterisks indicate the points of attachment of X to the respective neighbouring atoms within ring A;
wherein a wavy line indicates the point of attachment to L; and
an asterisk indicates the point of attachment to the carbonyl group of formula (I).

In a particularly preferred embodiment, the present invention provides a compound of formula (I) as described herein, or a pharmaceutically acceptable salt thereof, wherein:
A is

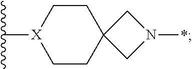

and
X and L, taken together, form a group

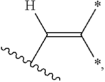

wherein the wavy line indicates the point of attachment of L to R², and the two asterisks indicate the points of attachment of X to the respective neighbouring atoms within ring A;
wherein a wavy line indicates the point of attachment to L; and
an asterisk indicates the point of attachment to the carbonyl group of formula (I).

In one embodiment, the present invention provides a compound of formula (I) as described herein, or a pharmaceutically acceptable salt thereof, wherein the compound of formula (I) is selected from:
(4aR,8aS)-6-(6-((4,4,5,5-tetramethyl-1,3,2-dioxaborolan-2-yl)methylene)-2-azaspiro[3.3]heptane-2-carbonyl)hexahydro-2H-pyrido[4,3-b][1,4]oxazin-3(4H)-one;
(4aR,8aS)-6-(3-(((2-oxo-2H-chromen-7-yl)oxy)methyl)azetidine-1-carbonyl)hexahydro-2H-pyrido[4,3-b][1,4]oxazin-3(4H)-one;
(4aR,8aS)-6-(3-(((2-oxo-2H-chromen-4-yl)oxy)methyl)azetidine-1-carbonyl)hexahydro-2H-pyrido[4,3-b][1,4]oxazin-3(4H)-one;
(4aR,8aS)-6-(6-((2-oxo-2H-chromen-4-yl)oxy)-2-azaspiro[3.3]heptane-2-carbonyl)hexahydro-2H-pyrido[4,3-b][1,4]oxazin-3(4H)-one;
(4aR,8aS)-6-(4-((7-methoxy-2-oxo-2H-chromen-4-yl)methyl)piperazine-1-carbonyl)hexahydro-2H-pyrido[4,3-b][1,4]oxazin-3(4H)-one;
(4aR,8aS)-6-(4-((7-nitrobenzo[c][1,2,5]oxadiazol-4-yl)amino)piperidine-1-carbonyl)hexahydro-2H-pyrido[4,3-b][1,4]oxazin-3(4H)-one;
(4aR,8aS)-6-(3-(((5,5-difluoro-5H-4λ⁴,5λ⁴-dipyrrolo[1,2-c:2′,1′-f][1,3,2]diazaborinin-10-yl)oxy)methyl)azetidine-1-carbonyl)hexahydro-2H-pyrido[4,3-b][1,4]oxazin-3(4H)-one;
(4aR,8aS)-6-(6-((5,5-difluoro-5H-4λ⁴,5λ⁴-dipyrrolo[1,2-c:2′,1′-f][1,3,2]diazaborinin-10-yl)oxy)-2-azaspiro[3.3]heptane-2-carbonyl)hexahydro-2H-pyrido[4,3-b][1,4]oxazin-3(4H)-one;
(4aR,8aS)-6-(4-((5,5-difluoro-5H-4λ⁴,5λ⁴-dipyrrolo[1,2-c:2′,1′-f][1,3,2]diazaborinin-10-yl)oxy)piperidine-1-carbonyl)hexahydro-2H-pyrido[4,3-b][1,4]oxazin-3(4H)-one;
(4aR,8aS)-6-(7-((5,5-difluoro-5H-4λ⁴,5λ⁴-dipyrrolo[1,2-c:2′,1′-f][1,3,2]diazaborinin-10-yl)oxy)-2-azaspiro[3.5]nonane-2-carbonyl)hexahydro-2H-pyrido[4,3-b][1,4]oxazin-3(4H)-one;
2-(6-((5,5-difluoro-5H-4λ⁴,5λ⁴-dipyrrolo[1,2-c:2′,1′-f][1,3,2]diazaborinin-10-yl)oxy)-2-azaspiro[3.3]heptane-2-carbonyl)-7-oxa-2,5-diazaspiro[3.4]octan-6-one;
(4aR,8aS)-6-(2-((5,5-difluoro-5H-4λ⁴,5λ⁴-dipyrrolo[1,2-c:2′,1′-f][1,3,2]diazaborinin-10-yl)oxy)-7-azaspiro[3.5]nonane-7-carbonyl)hexahydro-2H-pyrido[4,3-b][1,4]oxazin-3(4H)-one;
2-(2-((5,5-difluoro-5H-4λ4,5λ4-dipyrrolo[1,2-c:2′,1′-f][1,3,2]diazaborinin-10-yl)oxy)-7-azaspiro[3.5]nonane-7-carbonyl)-7-oxa-2,5-diazaspiro[3.4]octan-6-one;
(4aR,8aS)-6-((1R,5S,6R)-6-(((5,5-difluoro-5H-4λ⁴,5λ⁴-dipyrrolo[1,2-c:2′,1′-f][1,3,2]diazaborinin-10-yl)oxy)methyl)-3-azabicyclo[3.1.0]hexane-3-carbonyl)hexahydro-2H-pyrido[4,3-b][1,4]oxazin-3(4H)-one;
(4aR,8aS)-6-(6-((5,5-difluoro-5H-4λ⁴,5λ⁴-dipyrrolo[1,2-c:2′,1′-f][1,3,2]diazaborinin-10-yl)methylene)-2-azaspiro[3.3]heptane-2-carbonyl)hexahydro-2H-pyrido[4,3-b][1,4]oxazin-3(4H)-one;
(4aR,8aS)-6-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one;
(4aR,8aS)-6-(4-((5,5-difluoro-5H-4λ⁴,5λ⁴-dipyrrolo[1,2-c:2′,1′-f][1,3,2]diazaborinin-10-yl)methylene)piperidine-1-carbonyl)hexahydro-2H-pyrido[4,3-b][1,4]oxazin-3(4H)-one;
(4aR,8aS)-6-(7-((5,5-difluoro-5H-4λ⁴,5λ⁴-dipyrrolo[1,2-c:2′,1′-f][1,3,2]diazaborinin-10-yl)methylene)-2-azaspiro[3.5]nonane-2-carbonyl)hexahydro-2H-pyrido[4,3-b][1,4]oxazin-3(4H)-one;
(4aR,8aS)-6-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonane-2-carbonyl)hexahydro-2H-pyrido[4,3-b][1,4]oxazin-3(4H)-one;
2-(7-((5,5-difluoro-5H-4λ⁴,5λ⁴-dipyrrolo[1,2-c:2′,1′-f][1,3,2]diazaborinin-10-yl)methylene)-2-azaspiro[3.5]nonane-2-carbonyl)-7-oxa-2,5-diazaspiro[3.4]octan-6-one;
2-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonane-2-carbonyl)-7-oxa-2,5-diazaspiro[3.4]octan-6-one;
2-(6-((5,5-difluoro-5H-4λ⁴,5λ⁴-dipyrrolo[1,2-c:2′,1′-f][1,3,2]diazaborinin-10-yl)methylene)-2-azaspiro[3.3]heptane-2-carbonyl)-7-oxa-2,5-diazaspiro[3.4]octan-6-one;
2-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)-7-oxa-2,5-diazaspiro[3.4]octan-6-one;
6-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)-2H-benzo[b][1,4]oxazin-3(4H)-one;
(6-(3-cyclopropyl-1H-1,2,4-triazol-1-yl)-2-azaspiro[3.3]heptan-2-yl)(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)methanone;
(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)(4-(5-methyloxazolo[4,5-b]pyridin-2-yl)piperazin-1-yl)methanone;
(S)-1-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)pyrrolidine-3-carboxamide;
(R)-4-(3-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)-3-oxopropyl)oxazolidin-2-one;
(4aR,8aS)-6-(6-((5,5-difluoro-3-phenyl-5H-4λ⁴,5)⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one;
(4aR,8aS)-6-(6-((5,5-difluoro-3-(1H-pyrrol-2-yl)-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one;
(S)-(3-(1H-1,2,3-triazol-5-yl)pyrrolidin-1-yl)(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)methanone;
(S)-(3-(1H-1,2,3-triazol-5-yl)pyrrolidin-1-yl)(7-((5,5-difluoro-5H-4λ⁴,5λ⁴-dipyrrolo[1,2-c:2′,1′-f][1,3,2]diazaborinin-10-yl)methyl)-2-azaspiro[3.5]nonan-2-yl)methanone;
(6-(3-cyclopropyl-1H-1,2,4-triazol-1-yl)-2-azaspiro[3.3]heptan-2-yl)(7-((5,5-difluoro-5H-4λ⁴,5λ⁴-dipyrrolo[1,2-c:2′,1′-f][1,3,2]diazaborinin-10-yl)methyl)-2-azaspiro[3.5]nonan-2-yl)methanone;
6-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonane-2-carbonyl)-2H-benzo[b][1,4]oxazin-3(4H)-one;
(R)-4-(3-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonan-2-yl)-3-oxopropyl)oxazolidin-2-one;
(S)-1-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonane-2-carbonyl)pyrrolidine-3-carboxamide;
(7-((5,5-difluoro-5H-4λ⁴,5λ⁴-dipyrrolo[1,2-c:2′,1′-f][1,3,2]diazaborinin-10-yl)methyl)-2-azaspiro[3.5]nonan-2-yl)(4-(5-methyloxazolo[4,5-b]pyridin-2-yl)piperazin-1-yl)methanone;
(4aR,8aS)-6-(2-((5,5-difluoro-5H-4λ⁴,5λ⁴-dipyrrolo[1,2-c:2′,1′-f][1,3,2]diazaborinin-10-yl)methyl)-7-azaspiro[3.5]nonane-7-carbonyl)hexahydro-2H-pyrido[4,3-b][1,4]oxazin-3(4H)-one;
2-(2-((5,5-difluoro-5H-4λ⁴,5λ⁴-dipyrrolo[1,2-c:2′,1′-f][1,3,2]diazaborinin-10-yl)methyl)-7-azaspiro[3.5]nonane-7-carbonyl)-7-oxa-2,5-diazaspiro[3.4]octan-6-one;
(S)-(3-(1H-1,2,3-triazol-5-yl)pyrrolidin-1-yl)(2-((5,5-difluoro-5H-4λ⁴,5λ⁴-dipyrrolo[1,2-c:2′,1′-f][1,3,2]diazaborinin-10-yl)methyl)-7-azaspiro[3.5]nonan-7-yl)methanone;
(6-(3-cyclopropyl-1H-1,2,4-triazol-1-yl)-2-azaspiro[3.3]heptan-2-yl)(2-((5,5-difluoro-5H-4λ⁴,5λ⁴-dipyrrolo[1,2-c:2′,1′-f][1,3,2]diazaborinin-10-yl)methyl)-7-azaspiro[3.5]nonan-7-yl)methanone;
6-(2-((5,5-difluoro-5H-4λ⁴,5λ⁴-dipyrrolo[1,2-c:2′,1′-f][1,3,2]diazaborinin-10-yl)methyl)-7-azaspiro[3.5]nonane-7-carbonyl)-2H-benzo[b][1,4]oxazin-3(4H)-one;
(R)-4-(3-(2-((5,5-difluoro-5H-4λ⁴,5λ⁴-dipyrrolo[1,2-c:2′,1′-f][1,3,2]diazaborinin-10-yl)methyl)-7-azaspiro[3.5]nonan-7-yl)-3-oxopropyl)oxazolidin-2-one;
(S)-1-(2-((5,5-difluoro-5H-4λ⁴,5λ⁴-dipyrrolo[1,2-c:2′,1′-f][1,3,2]diazaborinin-10-yl)methyl)-7-azaspiro[3.5]nonane-7-carbonyl)pyrrolidine-3-carboxamide;
(2-((5,5-difluoro-5H-4λ⁴,5λ⁴-dipyrrolo[1,2-c:2′,1′-f][1,3,2]diazaborinin-10-yl)methyl)-7-azaspiro[3.5]nonan-7-yl)(4-(5-methyloxazolo[4,5-b]pyridin-2-yl)piperazin-1-yl)methanone; and
(6-(3-cyclopropyl-1H-1,2,4-triazol-1-yl)-2-azaspiro[3.3]heptan-2-yl)(6-((5,5-difluoro-3-(1H-pyrrol-2-yl)-5H-5λ⁴,6λ⁴-dipyrrolo[1,2-c:2′,1′-f][1,3,2]diazaborinin-10-yl)methyl)-2-azaspiro[3.3]heptan-2-yl)methanone.

In a preferred embodiment, the present invention provides a compound of formula (I) as described herein, or a pharmaceutically acceptable salt thereof, wherein the compound of formula (I) is selected from:
(4aR,8aS)-6-(6-((4,4,5,5-tetramethyl-1,3,2-dioxaborolan-2-yl)methylene)-2-azaspiro[3.3]heptane-2-carbonyl)hexahydro-2H-pyrido[4,3-b][1,4]oxazin-3(4H)-one;
(4aR,8aS)-6-(7-((5,5-difluoro-5H-4λ⁴,5λ⁴-dipyrrolo[1,2-c:2′,1′-f][1,3,2]diazaborinin-10-yl)oxy)-2-azaspiro[3.5]nonane-2-carbonyl)hexahydro-2H-pyrido[4,3-b][1,4]oxazin-3(4H)-one;
2-(6-((5,5-difluoro-5H-4λ⁴,5λ⁴-dipyrrolo[1,2-c:2′,1′-f][1,3,2]diazaborinin-10-yl)oxy)-2-azaspiro[3.3]heptane-2-carbonyl)-7-oxa-2,5-diazaspiro[3.4]octan-6-one;
2-(2-((5,5-difluoro-5H-4λ⁴,5λ⁴-dipyrrolo[1,2-c:2′,1′-f][1,3,2]diazaborinin-10-yl)oxy)-7-azaspiro[3.5]nonane-7-carbonyl)-7-oxa-2,5-diazaspiro[3.4]octan-6-one;
(4aR,8aS)-6-(6-((5,5-difluoro-5H-4λ⁴,5λ⁴-dipyrrolo[1,2-c:2′,1′-f][1,3,2]diazaborinin-10-yl)methylene)-2-azaspiro[3.3]heptane-2-carbonyl)hexahydro-2H-pyrido[4,3-b][1,4]oxazin-3(4H)-one;
(4aR,8aS)-6-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one;
(4aR,8aS)-6-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonane-2-carbonyl)hexahydro-2H-pyrido[4,3-b][1,4]oxazin-3(4H)-one;
2-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonane-2-carbonyl)-7-oxa-2,5-diazaspiro[3.4]octan-6-one;
2-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)-7-oxa-2,5-diazaspiro[3.4]octan-6-one;
6-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)-2H-benzo[b][1,4]oxazin-3(4H)-one;
(6-(3-cyclopropyl-1H-1,2,4-triazol-1-yl)-2-azaspiro[3.3]heptan-2-yl)(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)methanone;
(S)-1-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)pyrrolidine-3-carboxamide;
(R)-4-(3-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)-3-oxopropyl)oxazolidin-2-one;
(4aR,8aS)-6-(6-((5,5-difluoro-3-phenyl-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one; and
(4aR,8aS)-6-(6-((5,5-difluoro-3-(1H-pyrrol-2-yl)-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one.

In a particularly preferred embodiment, the present invention provides a compound of formula (I) as described herein, or a pharmaceutically acceptable salt thereof, wherein the compound of formula (I) is selected from:
(4aR,8aS)-6-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one;
(6-(3-cyclopropyl-1H-1,2,4-triazol-1-yl)-2-azaspiro[3.3]heptan-2-yl)(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)methanone; and
(4aR,8aS)-6-(6-((5,5-difluoro-3-(1H-pyrrol-2-yl)-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one.

In a particularly preferred embodiment, the present invention provides a compound of formula (I) as described herein, or a pharmaceutically acceptable salt thereof, wherein the compound of formula (I) is (4aR,8aS)-6-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one.

In a particularly preferred embodiment, the present invention provides a compound of formula (I) as described herein, or a pharmaceutically acceptable salt thereof, wherein the compound of formula (I) is (6-(3-cyclopropyl-1H-1,2,4-triazol-1-yl)-2-azaspiro[3.3]heptan-2-yl)(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)methanone.

In a particularly preferred embodiment, the present invention provides a compound of formula (I) as described herein, or a pharmaceutically acceptable salt thereof, wherein the compound of formula (I) is (4aR,8aS)-6-(6-((5,5-difluoro-3-(1H-pyrrol-2-yl)-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one.

In a particular embodiment, the present invention provides pharmaceutically acceptable salts of the compounds according to formula (I) as described herein. In a further particular embodiment, the present invention provides compounds according to formula (I) as described herein as free bases.

### Processes of Manufacturing

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates.

Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)—insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, NY. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

The following abbreviations are used in the present text:
AcOH=acetic acid, ACN=acetonitrile, Boc=tert-butyloxycarbonyl, CAS RN=chemical abstracts registration number, Cbz=benzyloxycarbonyl, Cs₂CO₃=cesium carbonate, CO=carbon monoxide, CuCl=copper(I) chloride, CuCN=copper(I) cyanide, CuI=copper(I) iodide, CuTC=Cu(I)2-thiophencarboxylate, DMAP=4-dimethylaminopyridine, DME=dimethoxyethane, DMEDA=N,N′-dimethylethylenediamine, DMF=N,N-dimethylformamide, DMP=Dess-Martin periodinane DIPEA=N,N-diisopropylethylamine, dppf=1,1 bis(diphenyl phosphino)ferrocene, EDC·HCl=N-(3-dimethylaminopropyl)-N′-ethylcarbodiimide hydrochloride, EI=electron impact, ESI=electrospray ionization, EtOAc=ethyl acetate, EtOH=ethanol, h=hour(s), FA=formic acid, H₂O=water, H₂SO₄=sulfuric acid, Hal=halogen, HATU=1-[bis(dimethylamino)methylene]-1H-1,2,3-triazolo[4,5-b]pyridinium-3-oxide hexafluorophosphate, HBTU=O-benzotriazole-N,N,N′,N′-tetramethyl-uronium-hexafluoro-phosphate, HCl=hydrogen chloride, HOBt=1-hydroxy-1H-benzotriazole; HPLC=high performance liquid chromatography, iPrMgCl=isopropylmagnesium chloride, I₂=iodine, IPA=2-propanol, (Ir[dF(CF₃)ppy]₂(dtbpy))PF₆=[4,4′-bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate, ISP=ion spray positive (mode), ISN=ion spray negative (mode), K₂CO₃=potassium carbonate, KHCO₃=potassium bicarbonate, KI=potassium iodide, KOH=potassium hydroxide, K₃PO₄=potassium phosphate tribasic, LiAlH₄ or LAH=lithium aluminium hydride, LiHMDS=lithium bis(trimethylsilyl)amide, LiGH=lithium hydroxide, MgSO₄=magnesium sulfate, min=minute(s), mL=milliliter, MPLC=medium pressure liquid chromatography, MS=mass spectrum, NaH=sodium hydride, NaHCO₃=sodium hydrogen carbonate, NaNO₂=sodium nitrite, NaOH=sodium hydroxide, Na₂CO₃=sodium carbonate, Na₂SO₄=sodium sulfate, Na₂S₂O₃=sodium thiosulfate, NBS=N-bromosuccinimide, nBuLi=n-butyllithium, NEt₃=triethylamine (TEA), NH₄Cl=ammonium chloride, NiCl₂ glyme=Nickel(II) chloride ethylene glycol dimethyl ether complex, NMP=N-methyl-2-pyrrolidone, OAc=Acetoxy, T₃P=propylphosphonic anhydride, P₂O₅=phosphorus pentoxide, PE=petroleum ether, PG=protective group, Pd—C=palladium on activated carbon, PdCl₂(dppf)-CH₂Cl₂=1,1′-bis(diphenylphosphino)ferrocene-palladium(II)dichloride dichloromethane complex, Pd₂(dba)₃=tris(dibenzylideneacetone)dipalladium(O), Pd(OAc)₂=palladium(II) acetate, Pd(OH)₂=palladium hydroxide, Pd(PPh₃)₄=tetrakis(triphenylphosphine)palladium(O), PTSA=p-toluenesulfonic acid, R=any group, RT=room temperature, SFC=Supercritical Fluid Chromatography, S-PHOS=2-dicyclohexylphosphino-2′,6′-dimethoxybiphenyl, T3P=propylphosphonic anhydride, TBAI=tetra butyl ammonium iodine, TEA=triethylamine, TFA=trifluroacetic acid, THF=tetrahydrofuran, TMEDA=N,N,N′,N′-tetramethylethylenediamine, ZnCl₂=zinc chloride, Xantphos=4,5-Bis(diphenylphosphino)-9,9-dimethylxanthene.

Compounds of formula I can be synthesized in analogy to literature procedures and/or as depicted for example in Scheme 1.

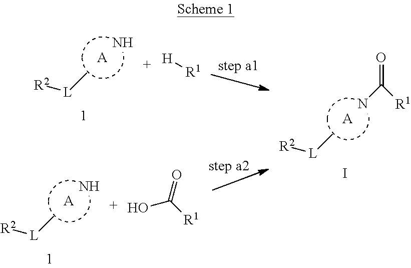

Accordingly, R¹ moieties that contain an N at the point of attachment to the remainder of formula I, are reacted with intermediates 1 in the presence of a urea forming reagent such as bis(trichloromethyl) carbonate using a suitable base and solvent such as, e.g. sodium bicarbonate in DCM, to give compounds of formula I (step al). Further urea forming reagents include but are not limited to phosgene, trichloromethyl chloroformate, (4-nitrophenyl)carbonate, 1,1′-carbonyldiimidazole or 1,1′-carbonyl-di-(1,2,4-triazole). Reactions of this type and the use of these reagents are widely described in literature (e.g. G. Sartori et al., Green Chemistry 2000, 2, 140). A person skilled in the art will acknowledge that the order of the addition of the reagents can be important in this type of reactions due to the reactivity and stability of the intermediary formed carbamoyl reagents, as well as for avoiding formation of undesired symmetrical urea by-products. R¹ moieties that contain a C at the point of attachment to the remainder of formula I are introduced as carboxyl acids that are coupled with intermediate amines 1 to form the respective amides (step a2). Amide couplings of this type can be accomplished by using one of the well-known coupling reagents such as DCC, HATU, EDCI, HOBt, TBTU, T3P, etc. and a base like Huenig's base, triethylamine or DMAP in a suitable solvent like N,N-dimethylformamide, DMA, DCM or 1-4-dioxane, preferably between 0° C. and room temperature.

Fluorescent probes of the general formula I can be obtained by a variety of synthetic routes, depending on their specific structure. Molecules by the structure Ta, where L=O, or probes containing the BODIPY scaffold with L=CH₂ Ic, or their precursors connected by a conjugated double bond Ib, as specified in the claim, may be synthesized as depicted for example in Scheme 2.

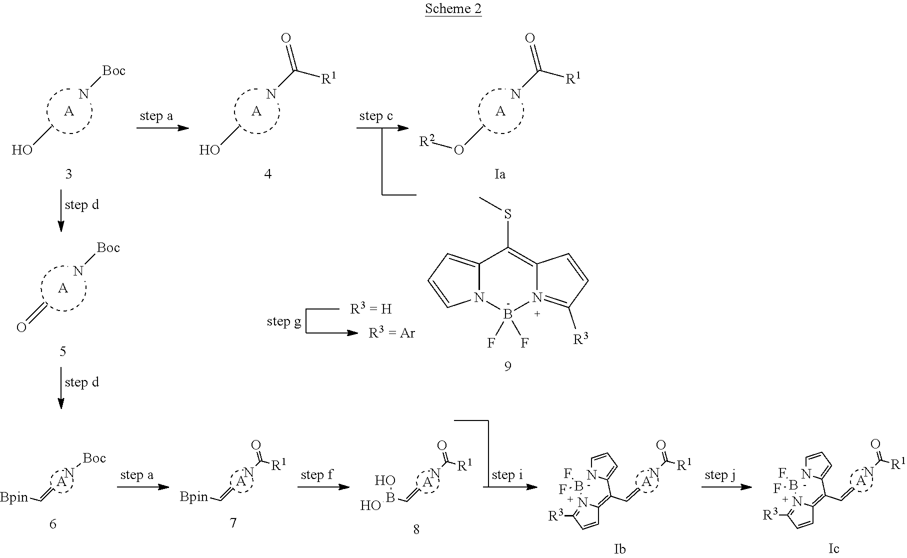

Intermediates 3 and 5 are either commercially available or can be prepared according to literature methods. Intermediates 3 can be attached to R¹ as described in scheme 1 (step a). The functional hydroxyl group can be utilized to attach R² groups by etherification reactions according to literature methods, e.g. under mitsunobo conditions with PPh3 and diethyl azodicarboxylate or in nucleophilic substitution (S_{N}) reactions (step c) to yield compounds Ta where L=0.

N-Boc protected alcohols 3 can be transformed to the respective ketones 5 by oxidation, e.g. by DMP, potassium dichromate or pyridinium chlorochromate in a suitable solvent such as DCM or toluene (step d). These can be readily transformed to the respective vinyl boronates 6 (e.g. vinyl boronic acid pinacol ester) as described in the literature (step e, Kovalenko et al. 2019). Boronic acids and its derivatives are known as versatile building blocks for transition metal mediated cross-coupling reactions. The N-boc protected vinyl boronates 6 can be selectively boc deprotected under widely used conditions such as TFA in DCM at ambient temperature, followed by attachment to R¹ moieties identical as described in scheme 1 step a. After hydrolysis of the boronates 7 to the respective boronic acids 8 by ammonium acetate buffered water/acetone mixtures and sodium periodate (step f) they can be attached to 8-Methylthio-BODIPY 9 and its 3 substituted analogues 10 and 11 by Liebeskind-Srogl reaction conditions with copper (I) salts such as Cu(I)TC, a Palladium catalyst such as, but not limited to, tris(dibenzylidenaceton)dipalladium and a palladium ligand such as tri-(2-furyl)-phosphin in a suitable solvent such as THE to give compounds of the formula Ib. This type of reaction has been described in the literature (Arroyo et al. 2011). The reduction of the conjugated double bond from compounds Ib to the methylene bridged BODIPY analogues Ic greatly enhances the fluorescent quantum yield. This property is essential to fluorescent probes of high quality as is known to those skilled in the art. This reduction can be achieved by widely used hydrogenation techniques with elemental hydrogen or a hydrogen generating reagent such as triethylsilane, in the presence of a suitable catalyst such as Pd or Pd(OH)₂ on charcoal in a suitable solvent such as MeOH, EtOH, EtOAc or mixtures thereof between 0° C. and reflux.

Molecules by the structure Id, where L=NH or NMe, may be synthesized as depicted for example in scheme 3.

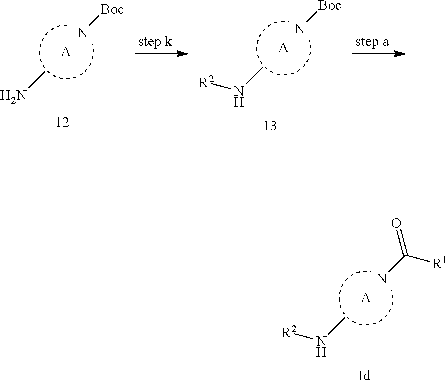

Commercially available N-Boc di-amines 12 can be attached to a fluorescent moiety R² by nucleophilic aromatic substitution, i.e. with 4-chlor-7-nitro-benzofurazan or 8-methylthio-BODIPY in absence or presence of an additional base such as triethylamine or potassium carbonate in a suitable solvent such as MeOH, DCM or THE between 0° C. and reflux. The modification of free NH groups by methylation is a known practice to those skilled in the art and can be performed on either intermediate 12, 13 or Id by methylation agents such as iodomethane or dimethyl sulfate with an additional base such as triethylamine, hunings base, potassium carbonate or cesium carbonate in an suitable solvent such as DMF or DMSO.

Correspondingly, molecules by the structure Ia, where L=O, may be synthesized as depicted for example in scheme 4 as an alternative to what is depicted in scheme 2.

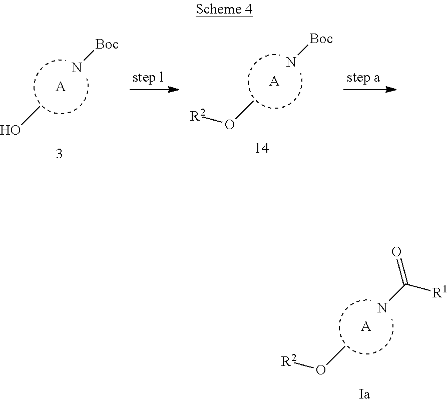

Commercially available intermediates 3 can be attached to R² by etherification reactions according to literature methods, e.g. under mitsunobo conditions with PPh₃ and diethyl azodicarboxylate or in nucleophilic substitution (S_{N}) reactions (step c) to yield Boc protected intermediates 14. Final molecules can be synthesized according to scheme 1 (step a).

In one aspect, the present invention provides a process of manufacturing the compounds of formula (I) as described herein, comprising:
(a) reacting a first amine 1,

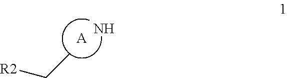

wherein A and R² are as defined herein;
with a second amine selected from:

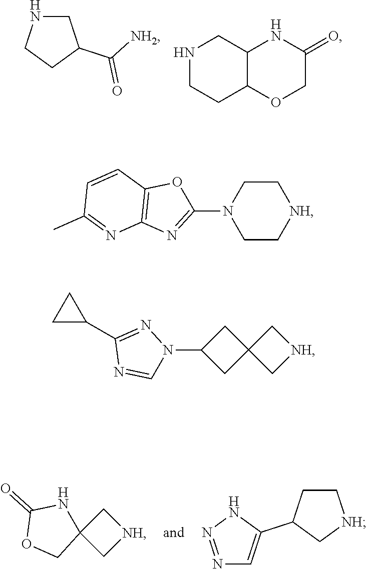 [SMILES: NC(=O)C1CCNC1 ; O=C1COC2CCNCC2N1 ; Cc1ccc2oc(N3CCNCC3)nc2n1 ; c1nc(C2CC2)nn1C1CC2(CNC2)C1 ; O=C1NC2(CNC2)CO1 ; c1nn[nH]c1C1CCNC1]

in the presence of a base and a urea forming reagent,
to form said compound of formula (I); or
(b) reacting an amine 1,

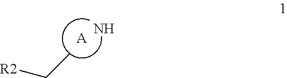

wherein A and R² are as defined herein;
with a carboxylic acid selected from:

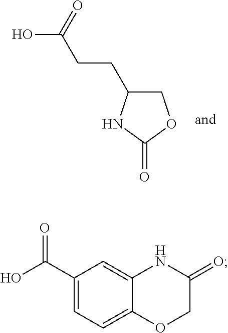 [SMILES: O=C(O)CCC1COC(=O)N1 ; O=C1COc2ccc(C(=O)O)cc2N1]

in the presence of a base and an amide coupling reagent,
to form said compound of formula (I).

In one embodiment, said urea forming reagent is selected from bis(trichloromethyl) carbonate, phosgene, trichloromethyl chloroformate, (4-nitrophenyl)carbonate, 1,1′-carbonyldiimidazole, and 1,1′-carbonyl-di-(1,2,4-triazole). Preferably, said urea forming reagent is bis(trichloromethyl) carbonate.

In one embodiment, said base in step (a) is sodium bicarbonate.

In one embodiment, step (a) is performed in a suitable solvent, preferably an aprotic solvent, more preferably DCM.

In one embodiment, said amide coupling reagent is selected from DCC, HATU, EDCI, HOBt, TBTU, and T3P.

In one embodiment, said base in step (b) is selected from Huenig's base, trimethylamine, and DMAP.

In one embodiment, step (b) is performed in a suitable solvent, preferably in a solvent selected from N,N-dimethylformamide, DMA, DCM, and 1-4-dioxane.

In one embodiment, step (b) is performed between 0° C. and room temperature.

In one aspect, the present invention provides a compound of formula (I) as described herein, when manufactured according to any one of the processes described herein.

### MAGL Inhibitory Activity

Compounds were profiled for MAGL inhibitory activity by determining the enzymatic activity by following the hydrolysis of the natural substrate 2-arachidonoylglycerol (2-AG) resulting in arachidonic acid, which can be followed by mass spectrometry. This assay is hereinafter abbreviated “2-AG assay”.

The 2-AG assay was carried out in 384 well assay plates (PP, Greiner Cat #784201) in a total volume of 20 μL. Compound dilutions were made in 100% DMSO (VWR Chemicals 23500.297) in a polypropylene plate in 3-fold dilution steps to give a final concentration range in the assay from 12.5 μM to 0.8 μM. 0.25 μL compound dilutions (100% DMSO) were added to 9 μL MAGL in assay buffer (50 mM TRIS (GIBCO, 15567-027), 1 mM EDTA (Fluka, 03690-100 mL), 0.01% (v/v) Tween. After shaking, the plate was incubated for 15 min at RT. To start the reaction, 10 μL 2-arachidonoylglycerol in assay buffer was added. The final concentrations in the assay was 50 μM MAGL and 8 μM 2-arachidonoylglyerol. After shaking and 30 min incubation at RT, the reaction was quenched by the addition of 40 μL of ACN containing 4 μM of d8-arachidonic acid. The amount of arachidonic acid was traced by an online SPE system (Agilent Rapidfire) coupled to a triple quadrupole mass spectrometer (Agilent 6460). A C18 SPE cartridge (G9205A) was used in an ACN/water liquid setup. The mass spectrometer was operated in negative electrospray mode following the mass transitions 303.1→259.1 for arachidonic acid and 311.1→267.0 for d8-arachidonic acid. The activity of the compounds was calculated based on the ratio of intensities [arachidonic acid/d8-arachidonic acid].

TABLE 1

| Example No | IC₅₀ hMAGL (nM) |
|---|---|
| 1 | 255.43 |
| 2 | 486.85 |
| 3 | 667.33 |
| 4 | 298.18 |
| 5 | 3311.60 |
| 6 | 3813.11 |
| 7 | 2587.44 |
| 8 | 79.29 |
| 9 | 6323.98 |
| 10 | 3.82 |
| 11 | 1.76 |
| 12 | 45.02 |
| 13 | 3.99 |
| 14 | 499.85 |
| 15 | 43.30 |
| 16 | 0.77 |
| 17 | 1683.16 |
| 18 | 0.64 |
| 19 | 2.35 |
| 20 | 0.67 |
| 21 | 0.69 |
| 22 | 12.58 |
| 23 | 1.26 |
| 24 | 7.50 |
| 25 | 0.34 |
| 26 | 17.17 |
| 27 | 1.98 |
| 28 | 1.01 |
| 29 | 0.66 |
| 30 | 0.05 |
| 31 | 0.59 |

In one aspect, the present invention provides compounds of formula (I) and their pharmaceutically acceptable salts or esters as described herein, wherein said compounds of formula (I) and their pharmaceutically acceptable salts or esters have IC₅₀'s for MAGL inhibition below 25 μM, preferably below 10 μM, more preferably below 5 M as measured in the MAGL assay described herein.

In one embodiment, compounds of formula (I) and their pharmaceutically acceptable salts or esters as described herein have IC₅₀ (MAGL inhibition) values between 0.000001 μM and 25 μM, particular compounds have IC₅₀ values between 0.000005 μM and 10 μM, further particular compounds have IC₅₀ values between 0.00005 μM and 5 μM, as measured in the MAGL assay described herein.

### Using the Compounds of the Invention

The compounds of formula (I) are fluorescent imaging probes with high affinity for MAGL. They may thus be used as high resolution tools to investigate localization, e.g. expression levels and protein distribution in health and disease, structure, dynamics and function of MAGL in living cells. They may also be applied e.g. in flow cytometry fluorescence-activated cell sorting (FACS) experiments or cellular trafficking studies using confocal live cell imaging.

In one aspect, the present invention provides a compound of formula (I) described herein, for use in monoacylglycerol lipase (MAGL) occupancy studies.

In a further aspect, the present invention provides a compound of formula (I) described herein, for use in diagnostic imaging of monoacylglycerol lipase (MAGL) in a mammal.

In a further aspect, the present invention provides a compound of formula (I) described herein, for use in generating monoacylglycerol lipase (MAGL) equilibrium and kinetic binding data.

In a further aspect, the present invention provides using a compound of formula (I) described herein in monoacylglycerol lipase (MAGL) occupancy studies.

In a further aspect, the present invention provides using a compound of formula (I) described herein in diagnostic imaging of monoacylglycerol lipase (MAGL) in a mammal.

In a further aspect, the present invention provides using a compound of formula (I) described herein for generating monoacylglycerol lipase (MAGL) equilibrium and kinetic binding data.

In a further aspect, the present invention provides a method of studying monoacylglycerol lipase (MAGL) occupancy, comprising contacting MAGL with a compound of formula (I) described herein.

In a further aspect, the present invention provides a method of diagnostic imaging of monoacylglycerol lipase (MAGL) in a mammal, comprising contacting MAGL with a compound of formula (I) described herein.

In a further aspect, the present invention provides a method of generating monoacylglycerol lipase (MAGL) equilibrium and kinetic binding data, comprising contacting MAGL with a compound of formula (I) described herein.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

### Example 1 (4aR,8aS)-6-(6-((4,4,5,5-tetramethyl-1,3,2-dioxaborolan-2-yl)methylene)-2-azaspiro[3.3]heptane-2-carbonyl)hexahydro-2H-pyrido[4,3-b][1,4]oxazin-3(4H)-one

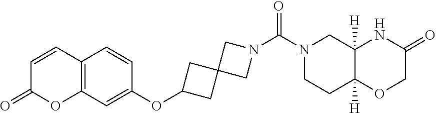 [SMILES: O=C1CO[C@H]2CCN(C(=O)N3CC4(CC(Oc5ccc6ccc(=O)oc6c5)C4)C3)C[C@H]2N1]

### Step 1) tert-butyl 6-((2-oxo-2H-chromen-7-yl)oxy)-2-azaspiro[3.3]heptane-2-carboxylate

Triphenylphosphine and DIAD were dissolved in 5 mL of anhydrous THF under N₂ at 0° C. for 15 minutes, then 7-hydroxy-2H-chromen-2-one (162 mg, 1.0 mmol) (CAS: 93-35-6) and the tert-butyl 6-hydroxy-2-azaspiro[3.3]heptane-2-carboxylate (107 mg, 0.5 mmol) (CAS: 1147557-97-8) each in 2 mL THE were added sequentially at 0° C. The mixture was allowed to warm to r.t. and was stirred for 24 h. The mixture was concentrated and purified via silica gel chromatography with a gradient elution of 0 to 50% ethylacetate in cyclohexane to give the title compound as a colorless solid (110 mg, 62%). LC-MS (ESI): m/z=358.2 [M+H]⁺.

### Step a1) (4aR,8aS)-6-(6-((4,4,5,5-tetramethyl-1,3,2-dioxaborolan-2-yl)methylene)-2-azaspiro[3.3]heptane-2-carbonyl)hexahydro-2H-pyrido[4,3-b][1,4]oxazin-3(4H)-one

tert-butyl 6-((2-oxo-2H-chromen-7-yl)oxy)-2-azaspiro[3.3]heptane-2-carboxylate (28.6 mg, 0.08 mmol) was boc deprotected by stirring in 5 ml TFA:DCM (1:4) at r.t. for 3 h. To remove traces of TFA it was twice coevaporated with 5 mL of toluene under reduced pressure. To a mixture of (4aR,8aS)-3-oxooctahydro-2H-pyrido[4,3-b][1,4]oxazin-6-ium 2,3-bis((4-methylbenzoyl)oxy)succinate (59.5 mg, 0.08 mmol), ACN (1 mL) and TEA (101 μL, 0.56 mmol), CDT (13.1 mg, 0.08 mmol) was added and the mixture stirred at room temperature for 2 h. The deprotected amine TFA salt was dissolved in 1 mL ACN and added dropwise to the mixture which was then warmed to 50° C. for 3 h. The cooled rection mixture was diluted with 20 mL EtOAc, washed with 10 mL saturated NaHCO₃ solution and 10 mL saturated NaCl solution. The organic layer was dried over NaSO₄, filtrated, and concentrated under reduced pressure to give a colorless residue that was purified via RP-HPLC (15-85% ACN:H₂O+0.1% TFA) to give the title compound as a colorless solid (15 mg, 43%). LC-MS (ESI): m/z=440.3 [M+H]⁺.

### Example 2 (4aR,8aS)-6-(3-(((2-oxo-2H-chromen-7-yl)oxy)methyl)azetidine-1-carbonyl)hexahydro-2H-pyrido[4,3-b][1,4]oxazin-3(4H)-one

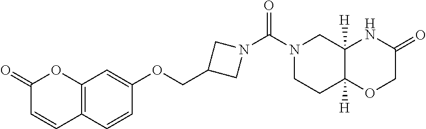 [SMILES: O=C1CO[C@H]2CCN(C(=O)N3CC(COc4ccc5ccc(=O)oc5c4)C3)C[C@H]2N1]

### Step 1) tert-butyl 3-(((2-oxo-2H-chromen-7-yl)oxy)methyl)azetidine-1-carboxylate

In analogy to the procedure described in example 11) 7-hydroxy-2H-chromen-2-one (CAS: 93-35-6) and tert-butyl 3-(hydroxymethyl)azetidine-1-carboxylate (CAS: 142253-56-3) were condensed to give the title compound as a colorless amorphous solid. LC-MS (ESI): m/z=332.3 [M+H]⁺.

### Step a1) (4aR,8aS)-6-(3-(((2-oxo-2H-chromen-7-yl)oxy)methyl)azetidine-1-carbonyl)hexahydro-2H-pyrido[4,3-b][1,4]oxazin-3(4H)-one

In analogy to the procedure described in example 1 a) (4aR,8aS)-hexahydro-2H-pyrido[4,3-b][1,4]oxazin-3(4H)-one (CAS: 2377107-31-6) and tert-butyl 3-(((2-oxo-2H-chromen-7-yl)oxy)methyl)azetidine-1-carboxylate were condensed to give the title compound. LC-MS (ESI): m/z=414.3 [M+H]⁺.

### Example 3 (4aR,8aS)-6-(3-(((2-oxo-2H-chromen-4-yl)oxy)methyl)azetidine-1-carbonyl)hexahydro-2H-pyrido[4,3-b][1,4]oxazin-3(4H)-one

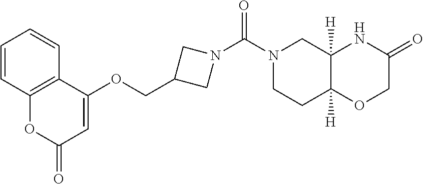 [SMILES: O=C1CO[C@H]2CCN(C(=O)N3CC(COc4cc(=O)oc5ccccc45)C3)C[C@H]2N1]

### Step 1) tert-butyl 3-(((2-oxo-2H-chromen-4-yl)oxy)methyl)azetidine-1-carboxylate

In analogy to the procedure described in example 11) 4-hydroxy-2H-chromen-2-one (CAS: 1076-38-6) and tert-butyl 3-(hydroxymethyl)azetidine-1-carboxylate (CAS: 142253-56-3) were condensed to give the title compound as a colorless amorphous solid. LC-MS (ESI): m/z=332.3 [M+H]⁺.

### Step a1) (4aR,8aS)-6-(3-(((2-oxo-2H-chromen-4-yl)oxy)methyl)azetidine-1-carbonyl)hexahydro-2H-pyrido[4,3-b][1,4]oxazin-3(4H)-one

In analogy to the procedure described in example 1 a(4aR,8aS)-hexahydro-2H-pyrido[4,3-b][1,4]oxazin-3(4H)-one (CAS: 2377107-31-6) and tert-butyl 3-(((2-oxo-2H-chromen-4-yl)oxy)methyl)azetidine-1-carboxylate were condensed to give the title compound as a colorless solid. LC-MS (ESI): m/z=414.3 [M+H]⁺.

### Example 4 (4aR,8aS)-6-(6-((2-oxo-2H-chromen-4-yl)oxy)-2-azaspiro[3.3]heptane-2-carbonyl)hexahydro-2H-pyrido[4,3-b][1,4]oxazin-3(4H)-one

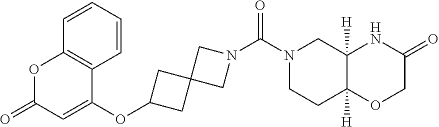 [SMILES: O=C1CO[C@H]2CCN(C(=O)N3CC4(CC(Oc5cc(=O)oc6ccccc56)C4)C3)C[C@H]2N1]

### Step 1) tert-butyl 6-((2-oxo-2H-chromen-4-yl)oxy)-2-azaspiro[3.3]heptane-2-carboxylate

In analogy to the procedure described in example 11) 4-hydroxy-2H-chromen-2-one (CAS: 1076-38-6) tert-butyl 6-hydroxy-2-azaspiro[3.3]heptane-2-carboxylate (CAS: 1147557-97-8) were condensed to give the title compound as a colorless amorphous solid.

LC-MS (ESI): m/z=358.3 [M+H]⁺.

### Step a1) (4aR,8aS)-6-(6-((2-oxo-2H-chromen-7-yl)oxy)-2-azaspiro[3.3]heptane-2-carbonyl)hexahydro-2H-pyrido[4,3-b][1,4]oxazin-3(4H)-one

In analogy to the procedure described in example 1 a(4aR,8aS)-hexahydro-2H-pyrido[4,3-b][1,4]oxazin-3(4H)-one (CAS: 2377107-31-6) and tert-butyl 3-(((2-oxo-2H-chromen-4-yl)oxy)methyl)azetidine-1-carboxylate were condensed to give the title compound as a colorless solid. LC-MS (ESI): m/z=440.2 [M+H]⁺.

### Example 5 (4aR,8aS)-6-(4-((7-methoxy-2-oxo-2H-chromen-4-yl)methyl)piperazine-1-carbonyl)hexahydro-2H-pyrido[4,3-b][1,4]oxazin-3(4H)-one

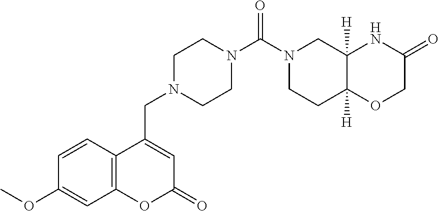 [SMILES: COc1ccc2c(CN3CCN(C(=O)N4CC[C@@H]5OCC(=O)N[C@@H]5C4)CC3)cc(=O)oc2c1]

### Step 1) tert-butyl 4-((7-methoxy-2-oxo-2H-chromen-4-yl)methyl)piperazine-1-carboxylate

A solution of 4-(bromomethyl)-7-methoxy-2H-chromen-2-one (134.5 mg, 0.5 mmol)(CAS: 35231-44-8) in DMF (1.25 mL) was treated with tert-butyl piperazine-1-carboxylate (186.3 mg, 1.0 mmol)(CAS: 57260-71-6), and the reaction mixture was stirred at room temperature for 3 h followed by dilution with water (5 mL). The aqueous layer was extracted with EtOAc (3×5 mL) and combined organic layers were dried over MgSO₄. Concentration of the organic layer under reduced pressure provided crude product, which was used in the following step without further purification. To an above obtained crude was added DCM/TFA (4:1, 5 mL) and the reaction mixture was stirred at room temperature for 3 h. To the reaction mixture was added a saturated solution of NaHCO₃ (4 mL) and extracted with EtOAc (6 mL). The organic layer was dried over MgSO₄ and then concentrated to give a crude product, that was purified via silica gel chromatography with gradient elution of 0 to 10% MeOH in DCM to give 7-methoxy-4-(piperazin-1-ylmethyl)-2H-chromen-2-one 2,2,2-trifluoroacetate (121 mg, 88% over 2 steps) as a colorless solid. LC-MS (ESI): m/z=275.1 [M+H]⁺.

### Step a1) (4aR,8aS)-6-(4-((7-methoxy-2-oxo-2H-chromen-4-yl)methyl)piperazine-1-carbonyl)hexahydro-2H-pyrido[4,3-b][1,4]oxazin-3(4H)-one

The boc deprotected intermediate was condensed with (4aR,8aS)-hexahydro-2H-pyrido[4,3-b][1,4]oxazin-3(4H)-one (CAS: 2377107-31-6) in analogy to the procedure described in example 1 a), to give the title compound as a colorless amorphous solid. LC-MS (ESI): 457.2 ([M+H]⁺).

### Example 6 (4aR,8aS)-6-(4-((7-nitrobenzo[c][1,2,5]oxadiazol-4-yl)amino)piperidine-1-carbonyl)hexahydro-2H-pyrido[4,3-b][1,4]oxazin-3(4H)-one

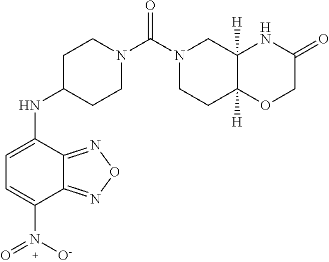

### Step 1) tert-butyl 4-((7-nitrobenzo[c][1,2,5]oxadiazol-4-yl)amino)piperidine-1-carboxylate

To a solution of tert-butyl 4-aminopiperidine-1-carboxylate (200 mg, 1.0 mmol)(CAS: 87120-72-7) in anhydrous ACN (2 mL) sodium hydrogencarbonate (252 mg, 3.0 mmol) was added, followed by 4-chloro-7-nitrobenzo[c][1,2,5]oxadiazole (200 mg, 1.0 mmol)(CAS: 10199-89-0) in 1 mL ACN. The mixture was stirred in darkness for 18 h at ambient temperature before being filtrated and concentrated to give a dark colored crude product (300 mg, 83%) that was used without further purification. LC-MS (ESI): m/z=364.2 [M+H]⁺.

### Step a1) (4aR,8aS)-6-(4-((7-nitrobenzo[c][1,2,5]oxadiazol-4-yl)amino)piperidine-1-carbonyl)hexahydro-2H-pyrido[4,3-b][1,4]oxazin-3(4H)-one

In analogy to the procedure described in example 1 a(4aR,8aS)-hexahydro-2H-pyrido[4,3-b][1,4]oxazin-3(4H)-one (CAS: 2377107-31-6) and tert-butyl 4-((7-nitrobenzo[c][1,2,5]oxadiazol-4-yl)amino)piperidine-1-carboxylate were condensed to give the title compound as an orange amorphous solid. LC-MS (ESI): m/z=468.1 [M+Na]⁺.

### Example 7 (4aR,8aS)-6-(3-(((5,5-difluoro-5H-4λ⁴,5λ⁴-dipyrrolo[1,2-c:2,1′-f][1,3,2]diazaborinin-10-yl)oxy)methyl)azetidine-1-carbonyl)hexahydro-2H-pyrido[4,3-b][1,4]oxazin-3(4H)-one

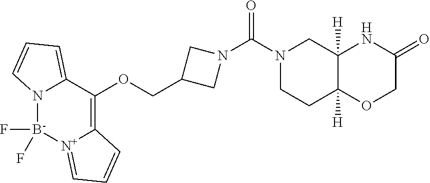 [SMILES: O=C1CO[C@H]2CCN(C(=O)N3CC(COC4=C5C=CC=[N+]5[B-](F)(F)n5cccc54)C3)C[C@H]2N1]

### Step a1) (4aR,8aS)-6-(3-(hydroxymethyl)azetidine-1-carbonyl)hexahydro-2H-pyrido[4,3-b][1,4]oxazin-3(4H)-one

tert-butyl 3-(hydroxymethyl)azetidine-1-carboxylate (15 mg, 0.08 mmol)(CAS: 142253-56-3) was boc deprotected by stirring in 5 ml TFA:DCM (1:4) at r.t. for 3 h. To remove traces of TFA it was twice coevaporated with 5 mL of toluene under reduced pressure. To a mixture of (4aR,8aS)-3-oxooctahydro-2H-pyrido[4,3-b][1,4]oxazin-6-ium 2,3-bis((4-methylbenzoyl)oxy)succinate (CAS: 2377107-31-6) (59.5 mg, 0.08 mmol), ACN (1 mL) and TEA (101 μL, 0.56 mmol), CDT (13.1 mg, 0.08 mmol) was added and the mixture stirred at room temperature for 2 h. The deprotected amine TFA salt was dissolved in 1 mL ACN and added dropwise to the mixture which was then warmed to 50° C. for 18 h. The cooled reaction mixture concentrated under reduced pressure and the residue was purified via RP-HPLC (5-75% ACN:H₂O+0.1% TFA) to give a colorless amorphous solid (12 mg, 56%). LC-MS (ESI): m/z=270.1 [M+H]⁺ and 292.1 [M+Na]⁺.

### Step 1) (4aR,8aS)-6-(3-(((5,5-difluoro-5H-4λ⁴,5λ⁴-dipyrrolo[1,2-c:2′,1′-f][1,3,2]diazaborinin-10-yl)oxy)methyl)azetidine-1-carbonyl)hexahydro-2H-pyrido[4,3-b][1,4]oxazin-3(4H)-one

[2-[(Methylthio)(2H-pyrrol-2-ylidene)methyl]-1H-pyrrole](difluoroborane) (CAS: 892505-41-8) (7.1 mg, 30 μmol) was dissolved in 1.5 mL anhydrous ACN in a dried schlenk tube und was purged with nitrogen for 5 minutes. CuTC (5.7 mg, 30 μmol) and (4aR,8aS)-6-(3-(hydroxymethyl)azetidine-1-carbonyl)hexahydro-2H-pyrido[4,3-b][1,4]oxazin-3(4H)-one were added and the mixture stirred for 5 min, before sodium carbonate (3.2 mg, 30 μmol) was added. The mixture was heated to 55° C. for 22 h. The mixture was concentrated under reduced pressure before being purified via RP-HPLC (15 to 85% ACN:H₂O+0.1% TFA) to give the title compound as an orange powder with green fluorescence in solution (2.4 mg, 17%). LC-MS (ESI): m/z=440.1 [M−F]⁺

### Example 8 (4aR,8aS)-6-(6-((5,5-difluoro-5H-4λ⁴, 5λ⁴-dipyrrolo[1,2-c:2′,1′-f][1,3,2]diazaborinin-10-yl)oxy)-2-azaspiro[3.3]heptane-2-carbonyl)hexahydro-2H-pyrido[4,3-b][1,4]oxazin-3(4H)-one

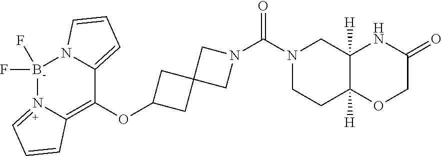

### Step a1) (4aR,8aS)-6-(6-hydroxy-2-azaspiro[3.3]heptane-2-carbonyl)hexahydro-2H-pyrido[4,3-b][1,4]oxazin-3(4H)-one

In analogy to the procedure described in example 7 al) tert-butyl 6-hydroxy-2-azaspiro[3.3]heptane-2-carboxylate (CAS: 1147557-97-8) and (4aR,8aS)-hexahydro-2H-pyrido[4,3-b][1,4]oxazin-3(4H)-one (CAS: 2377107-31-6) were condensed to give the title compound as a colorless amorphous solid. LC-MS (ESI): m/z=296.1 [M+H]⁺.

### Step 1) (4aR,8aS)-6-(6-((5,5-difluoro-5H-4λ⁴,5λ⁴-dipyrrolo[1,2-c:2′,1′-f][1,3,2]diazaborinin-10-yl)oxy)-2-azaspiro[3.3]heptane-2-carbonyl)hexahydro-2H-pyrido[4,3-b][1,4]oxazin-3(4H)-one

In analogy to the procedure described in example 7 1) (4aR,8aS)-6-(6-hydroxy-2-azaspiro[3.3]heptane-2-carbonyl)hexahydro-2H-pyrido[4,3-b][1,4]oxazin-3(4H)-one and [2-[(Methylthio)(2H-pyrrol-2-ylidene)methyl]-1H-pyrrole](difluoroborane) (CAS: 892505-41-8) were condensed to give the title compound as an orange powder with green fluorescence in solution. LC-MS (ESI): m/z=466.1 [M−F]⁺.

### Example 9 (4aR,8aS)-6-(4-((5,5-difluoro-5H-4λ⁴, 5λ⁴-dipyrrolo[1,2-c:2′,1′-f][1,3,2]diazaborinin-10-yl)oxy)piperidine-1-carbonyl)hexahydro-2H-pyrido[4,3-b][1,4]oxazin-3(4H)-one

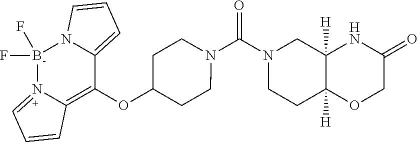

### Step a1 (4aR,8aS)-6-(4-hydroxypiperidine-1-carbonyl)hexahydro-2H-pyrido[4,3-b][1,4]oxazin-3(4H)-one

In analogy to the procedure described in example 7 al) tert-butyl 4-hydroxypiperidine-1-carboxylate (CAS: 109384-19-2) and (4aR,8aS)-hexahydro-2H-pyrido[4,3-b][1,4]oxazin-3(4H)-one (CAS: 2377107-31-6) were condensed to give the title compound as a colorless amorphous solid. LC-MS (ESI): m/z=284.1 [M+H]f.

### Step 1) (4aR,8aS)-6-(4-((5,5-difluoro-5H-4λ⁴,5λ⁴-dipyrrolo[1,2-c:2′,1′-f][1,3,2]diazaborinin-10-yl)oxy)piperidine-1-carbonyl)hexahydro-2H-pyrido[4,3-b][1,4]oxazin-3(4H)-one

In analogy to the procedure described in example 7 1) (4aR,8aS)-6-(4-hydroxypiperidine-1-carbonyl)hexahydro-2H-pyrido[4,3-b][1,4]oxazin-3(4H)-one and [2-[(Methylthio)(2H-pyrrol-2-ylidene)methyl]-1H-pyrrole](difluoroborane) (CAS: 892505-41-8) were condensed to give the title compound as an orange powder with green fluorescence in solution. LC-MS (ESI): m/z=454.2 [M−F]⁺.

### Example 10 (4aR,8aS)-6-(7-((5,5-difluoro-5H-4λ⁴, 5λ⁴-dipyrrolo[1,2-c:2,1′-f][1,3,2]diazaborinin-10-yl)oxy)-2-azaspiro[3.5]nonane-2-carbonyl)hexahydro-2H-pyrido[4,3-b][1,4]oxazin-3(4H)-one

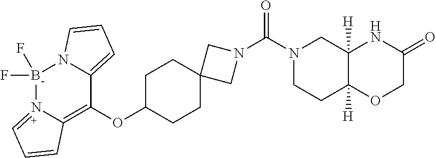

### Step a1) (4aR,8aS)-6-(7-hydroxy-2-azaspiro[3.5]nonane-2-carbonyl)hexahydro-2H-pyrido[4,3-b][1,4]oxazin-3(4H)-one

In analogy to the procedure described in example 7 al) tert-butyl 7-hydroxy-2-azaspiro[3.5]nonane-2-carboxylate (CAS: 1363383-18-9) and (4aR,8aS)-hexahydro-2H-pyrido[4,3-b][1,4]oxazin-3(4H)-one (CAS: 2377107-31-6) were condensed to give the title compound as a colorless amorphous solid. LC-MS (ESI): m/z=324.1 [M+H]⁺.

### Step 1) (4aR,8aS)-6-(7-((5,5-difluoro-5H-4λ⁴,5λ⁴-dipyrrolo[1,2-c:2′,1′-f][1,3,2]diazaborinin-10-yl)oxy)-2-azaspiro[3.5]nonane-2-carbonyl)hexahydro-2H-pyrido[4,3-b][1,4]oxazin-3(4H)-one

In analogy to the procedure described in example 7 1) (4aR,8aS)-6-(4-hydroxypiperidine-1-carbonyl)hexahydro-2H-pyrido[4,3-b][1,4]oxazin-3(4H)-one and [2-[(Methylthio)(2H-pyrrol-2-ylidene)methyl]-1H-pyrrole](difluoroborane) (CAS: 892505-41-8) were condensed to give the title compound as an orange powder with green fluorescence in solution. LC-MS (ESI): m/z=494.1 [M−F]*.

### Example 11 2-(6-((5,5-difluoro-5H-4λ⁴,5λ⁴-dipyrrolo[1,2-c:2′,1′-f][1,3,2]diazaborinin-10-yl)oxy)-2-azaspiro[3.3]heptane-2-carbonyl)-7-oxa-2,5-diazaspiro[3.4]octan-6-one

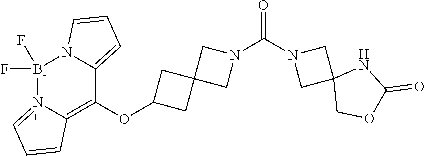

### Step a1) 2-(6-hydroxy-2-azaspiro[3.3]heptane-2-carbonyl)-7-oxa-2,5-diazaspiro[3.4]octan-6-one

In analogy to the procedure described in example 7 al) tert-butyl 6-hydroxy-2-azaspiro[3.3]heptane-2-carboxylate (CAS: 1147557-97-8) and 7-oxa-2,5-diazaspiro[3.4]octan-6-one (CAS: 1780174-72-2) were condensed to give the title compound as a colorless amorphous solid. LC-MS (ESI): m/z=268.1 [M+H]⁺.

### Step 1) 2-(6-((5,5-difluoro-5H-4λ⁴,5λ⁴-dipyrrolo[1,2-c:2′,1′-f][1,3,2]diazaborinin-10-yl)oxy)-2-azaspiro[3.3]heptane-2-carbonyl)-7-oxa-2,5-diazaspiro[3.4]octan-6-one

In analogy to the procedure described in example 7 1) 2-(6-hydroxy-2-azaspiro[3.3]heptane-2-carbonyl)-7-oxa-2,5-diazaspiro[3.4]octan-6-one and [2-[(Methylthio)(2H-pyrrol-2-ylidene)methyl]-1H-pyrrole](difluoroborane) (CAS: 892505-41-8) were condensed to give the title compound as an orange powder with green fluorescence in solution. LC-MS (ESI): m/z=438.1 [M−F]⁺.

### Example 12 (4aR,8aS)-6-(2-((5,5-difluoro-5H-4λ⁴, 5λ⁴-dipyrrolo[1,2-c:2,1′-f][1,3,2]diazaborinin-10-yl)oxy)-7-azaspiro[3.5]nonane-7-carbonyl)hexahydro-2H-pyrido[4,3-b][1,4]oxazin-3(4H)-one

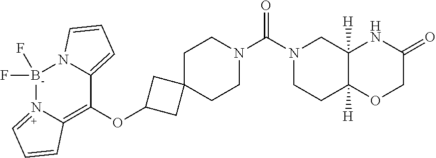

### Step a1) (4aR,8aS)-6-(2-hydroxy-7-azaspiro[3.5]nonane-7-carbonyl)hexahydro-2H-pyrido[4,3-b][1,4]oxazin-3(4H)-one

Tert-butyl 2-hydroxy-7-azaspiro[3.5]nonane-7-carboxylate (72.8 mg, 0.2 mmol)(CAS: 240401-28-9) was deprotected using 5 ml (1:4 TFA:DCM) at r.t. for 3 h. To remove traces of TFA it was twice coevaporated with 5 mL of toluene under reduced pressure To a ice cold suspension of triphosgene (42 mg, 0.14 mmol) (CAS: 32315-10-9) and sodium hydrogen carbonate (67 mg, 0.8 mmol) in 2 mL DCM was the deprotected intermediate in 1 mL anhydrous DCM. The mixture was allowed to warm to r.t overnight. The mixture was filtrated and the filtrate was added dropwise to a solution of (4aR,8aS)-3-oxooctahydro-2H-pyrido[4,3-b][1,4]oxazin-6-ium 2,3-bis((4-methylbenzoyl)oxy)succinate (149 mg, 0.2 mmol) (CAS: 2377107-31-6) and DIPEA (136 μL, 0.8 mmol) in 1 mL DCM at 0° C. The mixture was allowed to warm to r.t. and stirred for an additional 3 h. The mixture is concentrated in vacuo and purified via RP-HPLC (5 to 75% ACN:H₂O+0.1% TFA) to give the title compound as a colorless amorphous solid (32 mg, 49%). LC-MS (ESI): m/z=324.1 [M+H]⁺.

### Step 1) (4aR,8aS)-6-(2-((5,5-difluoro-5H-4λ⁴,5λ⁴-dipyrrolo[1,2-c:2′,1′-f][1,3,2]diazaborinin-10-yl)oxy)-7-azaspiro[3.5]nonane-7-carbonyl)hexahydro-2H-pyrido[4,3-b][1,4]oxazin-3(4H)-one

In analogy to the procedure described in example 7 1) (4aR,8aS)-6-(2-hydroxy-7-azaspiro[3.5]nonane-7-carbonyl)hexahydro-2H-pyrido[4,3-b][1,4]oxazin-3(4H)-one and [2-[(Methylthio)(2H-pyrrol-2-ylidene)methyl]-1H-pyrrole](difluoroborane) (CAS: 892505-41-8) were condensed to give the title compound as an orange powder with green fluorescence in solution. LC-MS (ESI): m/z=494.2 [M−F]⁺.

### Example 13 2-(2-((5,5-difluoro-5H-4λ⁴,5λ⁴-dipyrrolo[1,2-c:2′,1′-f][1,3,2]diazaborinin-10-yl)oxy)-7-azaspiro[3.5]nonane-7-carbonyl)-7-oxa-2,5-diazaspiro[3.4]octan-6-one

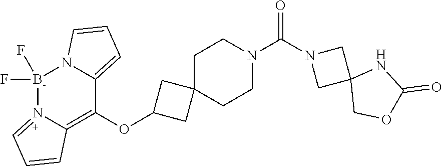

### Step a1) 2-(2-hydroxy-7-azaspiro[3.5]nonane-7-carbonyl)-7-oxa-2,5-diazaspiro[3.4]octan-6-one

In analogy to the procedure described in example 12 al) Tert-butyl 2-hydroxy-7-azaspiro[3.5]nonane-7-carboxylate (CAS: 240401-28-9) and 7-oxa-2,5-diazaspiro[3.4]octan-6-one (CAS: 1780174-72-2) were condensed to give the title compound as a colorless amorphous solid. LC-MS (ESI): m/z=296.1 [M+H]⁺.

### Step 1) 2-(2-((5,5-difluoro-5H-4λ⁴,5λ⁴-dipyrrolo[1,2-c:2′,1′-f][1,3,2]diazaborinin-10-yl)oxy)-7-azaspiro[3.5]nonane-7-carbonyl)-7-oxa-2,5-diazaspiro[3.4]octan-6-one

In analogy to the procedure described in example 7 1) 2-(2-hydroxy-7-azaspiro[3.5]nonane-7-carbonyl)-7-oxa-2,5-diazaspiro[3.4]octan-6-one and [2-[(Methylthio)(2H-pyrrol-2-ylidene)methyl]-1H-pyrrole](difluoroborane) (CAS: 892505-41-8) were condensed to give the title compound as an orange powder with green fluorescence in solution. LC-MS (ESI): m/z=466.2 [M−F]⁺.

### Example 14 (4aR,8aS)-6-((1R,5S,6R)-6-(((5,5-difluoro-5H-4λ⁴,5λ⁴-dipyrrolo[1,2-c:2′,1′-f][1,3,2]diazaborinin-10-yl)oxy)methyl)-3-azabicyclo[3.1.0]hexane-3-carbonyl)hexahydro-2H-pyrido[4,3-b][1,4]oxazin-3(4H)-one

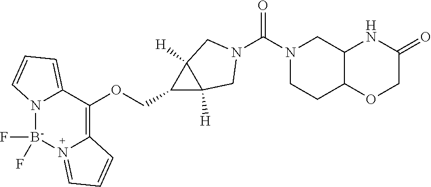

### Step a1) (4aR,8aS)-6-((1R,5S,6R)-6-(hydroxymethyl)-3-azabicyclo[3.1.0]hexane-3-carbonyl)hexahydro-2H-pyrido[4,3-b][1,4]oxazin-3(4H)-one

In analogy to the procedure described in example 12 al) tert-butyl (1R,5S,6r)-6-(hydroxymethyl)-3-azabicyclo[3.1.0]hexane-3-carboxylate (CAS: 419572-18-2) and (4aR,8aS)-hexahydro-2H-pyrido[4,3-b][1,4]oxazin-3(4H)-one (CAS: 2377107-31-6) were condensed to give the title compound as a colorless amorphous solid. LC-MS (ESI): m/z=296.2 [M+H]⁺.

### Step 1) (4aR,8aS)-6-((1R,5S,6R)-6-(((5,5-difluoro-5H-4λ⁴,5λ⁴-dipyrrolo[1,2-c:2′,1′-f][1,3,2]diazaborinin-10-yl)oxy)methyl)-3-azabicyclo[3.1.0]hexane-3-carbonyl)hexahydro-2H-pyrido[4,3-b][1,4]oxazin-3(4H)-one

In analogy to the procedure described in example 7 1) (4aR,8aS)-6-((1R,5S,6R)-6-(hydroxymethyl)-3-azabicyclo[3.1.0]hexane-3-carbonyl)hexahydro-2H-pyrido[4,3-b][1,4]oxazin-3(4H)-one and [2-[(Methylthio)(2H-pyrrol-2-ylidene)methyl]-1H-pyrrole](difluoroborane) (CAS: 892505-41-8) were condensed to give the title compound as an orange powder with green fluorescence in solution. LC-MS (ESI): m/z=466.1 [M-F]⁺.

### Example 15 (4aR,8aS)-6-(6-((5,5-difluoro-5H-4λ⁴, 5λ⁴-dipyrrolo[1,2-c:2,1′-f][1,3,2]diazaborinin-10-yl)methylene)-2-azaspiro[3.3]heptane-2-carbonyl)hexahydro-2H-pyrido[4,3-b][1,4]oxazin-3(4H)-one

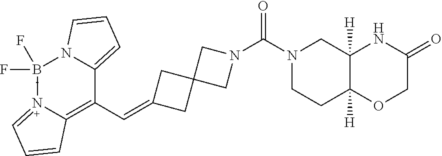

### Step e) tert-butyl 6-((4,4,5,5-tetramethyl-1,3,2-dioxaborolan-2-yl)methylene)-2-azaspiro[3.3]heptane-2-carboxylate

An oven dried vial with a magnetic stir bar was charged with 2,2,6,6-Tetramethylpiperidine (311 mg, 2.2 mmol), which was dissolved in anhydrous THE (20 mL) and cooled to −78° C. under nitrogen atmosphere. nBuLi (2.5 M in hexanes) (880 μL, 2.2 mmol) was added dropwise, and the reaction mixture was stirred at the same temperature for 30 min. Next, a solution of bis(4,4,5,5-tetramethyl-1,3,2-dioxaborolan-2-yl)methane (590 mg, 2.2 mmol) in THE (8 mL) was added dropwise. The reaction was allowed to stir for 5 minutes. Then tert-butyl 6-oxo-2-azaspiro[3.3]heptane-2-carboxylate (232 mg, 1.1 mmol) in THF (12 mL) was added dropwise over 5 min. The reaction vial was slowly allowed warm to r.t. overnight. Upon completion, the reaction was opened to air, and filtered through a silica plug with eluting diethyl ether. The mixture was concentrated under reduced pressure and adsorbed onto isolute. The product was obtained by silica gel chromatography 0 to 20% ethyl acetate in cyclohexane as a white amorphous solid. LC-MS (ESI): m/z=336.1 [M+H]⁺.

### Step a) (4aR,8aS)-6-(6-((4,4,5,5-tetramethyl-1,3,2-dioxaborolan-2-yl)methylene)-2-azaspiro[3.3]heptane-2-carbonyl)hexahydro-2H-pyrido[4,3-b][1,4]oxazin-3(4H)-one

tert-butyl 6-((4,4,5,5-tetramethyl-1,3,2-dioxaborolan-2-yl)methylene)-2-azaspiro[3.3]heptane-2-carboxylate (67 mg, 0.2 mmol) was boc deprotected by stirring in 5 ml TFA:DCM (1:4) at r.t. for 3 h. To remove traces of TFA it was twice coevaporated with 5 mL of toluene under reduced pressure. To a mixture of (4aR,8aS)-3-oxooctahydro-2H-pyrido[4,3-b][1,4]oxazin-6-ium 2,3-bis((4-methylbenzoyl)oxy)succinate (149 mg, 0.2 mmol) (CAS: 2377107-31-6), ACN (2 mL) and TEA (194 μL, 1.4 mmol), CDT was added and the mixture stirred at room temperature for 2 h. The deprotected amine TFA salt was dissolved in 1 mL ACN and added dropwise to the mixture which was then warmed to 50° C. for 3 h. The cooled rection mixture was diluted with 20 mL EtOAc, washed with 10 mL saturated NaHCO₃ solution and 10 mL saturated NaCl solution. The organic layer was dried over NaSO₄, filtrated, and concentrated under reduced pressure to give a colorless oil that was used without further purification. LC-MS (ESI): m/z=418.2 [M+H]⁺.

### Step f) ((2-((4aR,8aS)-3-oxooctahydro-2H-pyrido[4,3-b][1,4]oxazine-6-carbonyl)-2-azaspiro[3.3]heptan-6-ylidene)methyl)boronic acid

(4aR,8aS)-6-(6-((4,4,5,5-tetramethyl-1,3,2-dioxaborolan-2-yl)methylene)-2-azaspiro[3.3]heptane-2-carbonyl)hexahydro-2H-pyrido[4,3-b][1,4]oxazin-3(4H)-one (83.5 mg, 0.2 mmol), sodium periodate (214 mg, 1.0 mmol) and ammonium acetate (77 mg, 1.0 mmol) were dissolved in acetone/water 2:1 (4 mL, 0.05 M) and stirred for 24 h at ambient temperature until LC-MS indicated consumption of the starting material. Acetone was removed in vacuo and 4 mL of ACN/H₂O 1:1 were added and the mixture was filtrated. The filtrate was purified via RP-HPLC 5% to 75% ACN:H₂O (+0.1% TFA) to give the product as colorless amorphous solid after lyophilisation. LC-MS (ESI): m/z=336.1 [M+H]⁺.

### Step i) (4aR,8aS)-6-(6-((5,5-difluoro-5H-4λ⁴,5λ⁴-dipyrrolo[1,2-c:2′,1′-f][1,3,2]diazaborinin-10-yl)methylene)-2-azaspiro[3.3]heptane-2-carbonyl)hexahydro-2H-pyrido[4,3-b][1,4]oxazin-3(4H)-one

An oven-dry Schlenk tube, equipped with a stir bar, was charged with ((2-((4aR,8aS)-3-oxooctahydro-2H-pyrido[4,3-b][1,4]oxazine-6-carbonyl)-2-azaspiro[3.3]heptan-6-ylidene)methyl)boronic acid (33.5 mg, 0.1 mmol), [2-[(methylthio)(2H-pyrrol-2-ylidene)methyl]-1H-pyrrole](difluoroborane) (CAS: 892505-41-8) (24 mg, 0.1 mmol) and anhydrous THF (3 mL) under nitrogen. The stirred solution was sparged with nitrogen for 10 minutes, whereupon CuTC (57 mg, 0.3 mmol), Pd2(dba)3 (7 mg, 7.5 μmol), and TFP (5.2 mg, 22.5 μmol) were added under nitrogen. The reaction mixture was immersed into a pre-heated oil bath at 55° C. The reaction is monitored via LC-MS and upon consumption of the boronic acid after 1 h the heating was stopped. The reaction mixture was carefully concentrated and 4 mL of ACN:H₂O were added, filtered and purified via RP-HPLC (15 to 85% ACN:H₂O+0.1% TFA) to give a intensely red color amorphous solid (22.1 mg, 46%). LC-MS (ESI): m/z=482.2 [M+H]⁺ and 462.2 [M−F]⁺.

### Example 16 (4aR,8aS)-6-(6-((5,5-difluoro-5H-4λ⁴,5λ⁴-dipyrrolo[1,2-c:2,1′-f][1,3,2]diazaborinin-10-yl)methyl)-2-azaspiro[3.3]heptane-2-carbonyl)hexahydro-2H-pyrido[4,3-b][1,4]oxazin-3(4H)-one

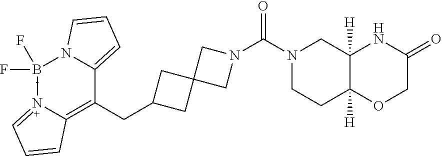

### Step j)

Example 15 was transformed to example 16 by the reduction of the olefinic double bond. Therefore (4aR,8aS)-6-(6-((5,5-difluoro-5H-4λ⁴,5λ⁴-dipyrrolo[1,2-c:2′,1′-f][1,3,2]diazaborinin-10-yl)methylene)-2-azaspiro[3.3]heptane-2-carbonyl)hexahydro-2H-pyrido[4,3-b][1,4]oxazin-3(4H)-one (24 mg, 50 μmol) was dissolved in 5 Ml anhydrous MeOH, purged with nitrogen before adding Pd/C (10 wt %, 5.3 mg, 5 μmol) and cooling to 0° C. Then triethylsilane (120 μL, 750 μmol) were added dropwise and the mixture was allowed to warm to r.t. over 30 min. Upon completion of the reaction, 0.5 mL water were added and the mixture was filtrated and concentrated under reduced pressure. The crude was purified via RP-HPLC (15 to 85% ACN:H₂O+0.1% TFA) to give the title compound as a red amorphous solid with green fluorescence in solution (19 mg, 79%). MS (ESI): m/z=484.2 [M+H]⁺ and 464.1 [M−F]⁺.

### Example 17 (4aR,8aS)-6-(4-((5,5-difluoro-5H-4λ⁴, 5λ⁴-dipyrrolo[1,2-c:2,1′-f][1,3,2]diazaborinin-O-yl)methylene)piperidine-1-carbonyl)hexahydro-2H-pyrido[4,3-b][1,4]oxazin-3(4H)-one

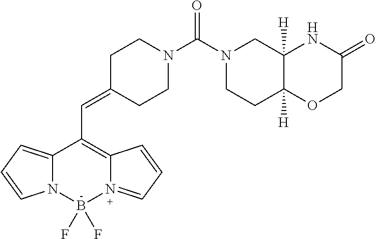

### Step e) tert-butyl 4-((4,4,5,5-tetramethyl-1,3,2-dioxaborolan-2-yl)methylene)piperidine-1-carboxylate

In analogy to the procedures described in example 15 e) tert-butyl 4-oxopiperidine-1-carboxylate (CAS: 79099-07-3) was converted to the respective vinyl boronate, which was obtained as a colorless solid. LC-MS (ESI): m/z=324.2 [M+H]⁺

### Step a1) (4aR,8aS)-6-(4-((4,4,5,5-tetramethyl-1,3,2-dioxaborolan-2-yl)methylene)piperidine-1-carbonyl)hexahydro-2H-pyrido[4,3-b][1,4]oxazin-3(4H)-one

In analogy to the procedures described in example 12 al) tert-butyl 4-((4,4,5,5-tetramethyl-1,3,2-dioxaborolan-2-yl)methylene)piperidine-1-carboxylate and (4aR,8aS)-hexahydro-2H-pyrido[4,3-b][1,4]oxazin-3(4H)-one (CAS: 2377107-31-6) were condensed to give the title compound as a colorless amorphous solid. LC-MS (ESI): m/z=406.2 [M+H]⁺

### Step f) ((1-((4aR,8aS)-3-oxooctahydro-2H-pyrido[4,3-b][1,4]oxazine-6-carbonyl)piperidin-4-ylidene)methyl)boronic acid

The vinyl boronate was hydrolyzed to the respective boronic acid in analogy to the procedures described in example 15 f) to give the title compound as colorless solid. LC-MS (ESI): m/z=324.1 [M+H]⁺

### Step i) (4aR,8aS)-6-(4-((5,5-difluoro-5H-4λ⁴,5λ⁴-dipyrrolo[1,2-c:2′,1′-f][1,3,2]diazaborinin-10-yl)methylene)piperidine-1-carbonyl)hexahydro-2H-pyrido[4,3-b][1,4]oxazin-3(4H)-one

In analogy to the procedures described in example 15 i) ((1-((4aR,8aS)-3-oxooctahydro-2H-pyrido[4,3-b][1,4]oxazine-6-carbonyl)piperidin-4-ylidene)methyl)boronic acid and [2-[(methylthio)(2H-pyrrol-2-ylidene)methyl]-1H-pyrrole](difluoroborane) (CAS: 892505-41-8) were coupled to give the title compound as an intensely red colored solid. LC-MS (ESI): m/z=450.1 [M−F]⁺

### Example 18 (4aR,8aS)-6-(7-((5,5-difluoro-5H-4λ⁴, 5λ⁴-dipyrrolo[1,2-c:2,1′-f][1,3,2]diazaborinin-10-yl)methylene)-2-azaspiro[3.5]nonane-2-carbonyl)hexahydro-2H-pyrido[4,3-b][1, 4]oxazin-3(4H)-one

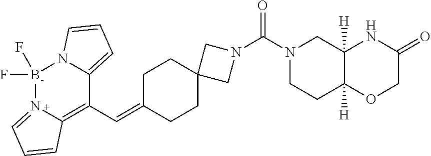

### Step e) tert-butyl 7-((4,4,5,5-tetramethyl-1,3,2-dioxaborolan-2-yl)methylene)-2-azaspiro[3.5]nonane-2-carboxylate

In analogy to the procedures described in example 15 e) tert-butyl 7-oxo-2-azaspiro[3.5]nonane-2-carboxylate (CAS:1363381-22-9) was converted to the respective vinyl boronate, which was obtained as a colorless solid. LC-MS (ESI): m/z=364.2 [M+H]⁺

### Step a1) (4aR,8aS)-6-(7-((4,4,5,5-tetramethyl-1,3,2-dioxaborolan-2-yl)methylene)-2-azaspiro[3.5]nonane-2-carbonyl)hexahydro-2H-pyrido[4,3-b][1,4]oxazin-3(4H)-one

In analogy to the procedures described in example 15 al) tert-butyl 7-((4,4,5,5-tetramethyl-1,3,2-dioxaborolan-2-yl)methylene)-2-azaspiro[3.5]nonane-2-carboxylate and (4aR,8aS)-hexahydro-2H-pyrido[4,3-b][1,4]oxazin-3(4H)-one (CAS: 2377107-31-6) were condensed to give the title compound as a colorless amorphous solid. LC-MS (ESI): m/z=446.2 [M+H]⁺

### Step f) ((2-((4aR,8aS)-3-oxooctahydro-2H-pyrido[4,3-b][1,4]oxazine-6-carbonyl)-2-azaspiro[3.5]nonan-7-ylidene)methyl)boronic acid

The vinyl boronate was hydrolyzed to the respective boronic acid in analogy to the procedures described in example 15 f) to give the title compound as colorless solid. LC-MS (ESI): m/z=364.1 [M+H]⁺

### Step i) (4aR,8aS)-6-(7-((5,5-difluoro-5H-4λ⁴,5λ⁴-dipyrrolo[1,2-c:2′,1′-f][1,3,2]diazaborinin-10-yl)methylene)-2-azaspiro[3.5]nonane-2-carbonyl)hexahydro-2H-pyrido[4,3-b][1,4]oxazin-3(4H)-one

In analogy to the procedures described in example 15 i) ((1-((4aR,8aS)-3-oxooctahydro-2H-pyrido[4,3-b][1,4]oxazine-6-carbonyl)piperidin-4-ylidene)methyl)boronic acid and [2-[(methylthio)(2H-pyrrol-2-ylidene)methyl]-1H-pyrrole](difluoroborane) (CAS: 892505-41-8) were coupled to give the title compound as an intensely red colored solid. LC-MS (ESI): m/z=490.1 [M−F]⁺

### Example 19 (4aR,8aS)-6-(7-((5,5-difluoro-5H-4λ⁴, 5λ⁴-dipyrrolo[1,2-c:2,1′-f][1,3,2]diazaborinin-10-yl)methyl)-2-azaspiro[3.5]nonane-2-carbonyl)hexahydro-2H-pyrido[4,3-b][1,4]oxazin-3(4H)-one

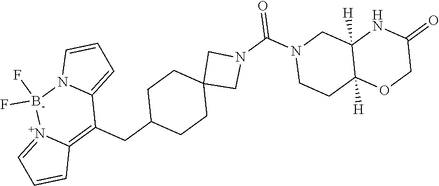

### Step j)

Example 18 was transformed to example 19 by the reduction of the olefinic double bond in analogy to the procedure described in example 16 j) to give a orange solid with green fluorescence in solution. LC-MS (ESI): m/z=492.1 [M−F]f.

### Example 20 2-(7-((5,5-difluoro-5H-4λ⁴,5λ⁴-dipyrrolo[1,2-c:2′,1′-f][1,3,2]diazaborinin-10-yl)methylene)-2-azaspiro[3.5]nonane-2-carbonyl)-7-oxa-2,5-diazaspiro[3.4]octan-6-one

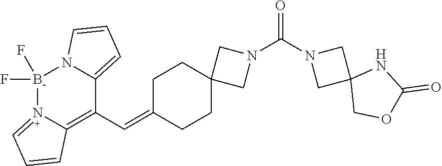

### Step a1) 2-(7-((4,4,5,5-tetramethyl-1,3,2-dioxaborolan-2-yl)methylene)-2-azaspiro[3.5]nonane-2-carbonyl)-7-oxa-2,5-diazaspiro[3.4]octan-6-one

In analogy to the procedures described in example 15 al) tert-butyl 7-((4,4,5,5-tetramethyl-1,3,2-dioxaborolan-2-yl)methylene)-2-azaspiro[3.5]nonane-2-carboxylate and 7-oxa-2,5-diazaspiro[3.4]octan-6-one (CAS: 1780174-72-2) were condensed to give the title compound as a colorless amorphous solid. LC-MS (ESI): m/z=418.1 [M+H]⁺

### Step f) ((2-(6-oxo-7-oxa-2,5-diazaspiro[3.4]octane-2-carbonyl)-2-azaspiro[3.5]nonan-7-ylidene)methyl)boronic acid

The vinyl boronate was hydrolyzed to the respective boronic acid in analogy to the procedures described in example 15 f) to give the title compound as colorless solid. LC-MS (ESI): m/z=336.1 [M+H]f

### Step i) 2-(7-((5,5-difluoro-5H-4λ⁴,5λ⁴-dipyrrolo[1,2-c:2′,1′-f][1,3,2]diazaborinin-10-yl)methylene)-2-azaspiro[3.5]nonane-2-carbonyl)-7-oxa-2,5-diazaspiro[3.4]octan-6-one

In analogy to the procedures described in example 15 i) ((1-((4aR,8aS)-3-oxooctahydro-2H-pyrido[4,3-b][1,4]oxazine-6-carbonyl)piperidin-4-ylidene)methyl)boronic acid and [2-[(methylthio)(2H-pyrrol-2-ylidene)methyl]-1H-pyrrole](difluoroborane) (CAS: 892505-41-8) were coupled to give the title compound as an intensely red colored solid. LC-MS (ESI): m/z=461.2 [M−F]⁺

### Example 21 2-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonane-2-carbonyl)-7-oxa-2,5-diazaspiro[3.4]octan-6-one

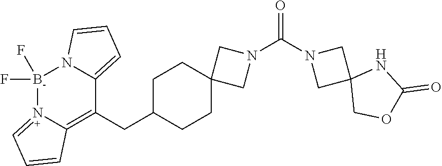

### Step j)

Example 20 was transformed to example 21 by the reduction of the olefinic double bond in analogy to the procedure described in example 16 j) to give a orange solid with green fluorescence in solution. LC-MS (ESI): m/z=463.2 [M−F]⁺.

### Example 22 2-(6-((5,5-difluoro-5H-4λ⁴,5λ⁴-dipyrrolo[1,2-c:2′,1′-f][1,3,2]diazaborinin-10-yl)methylene)-2-azaspiro[3.3]heptane-2-carbonyl)-7-oxa-2,5-diazaspiro[3.4]octan-6-one

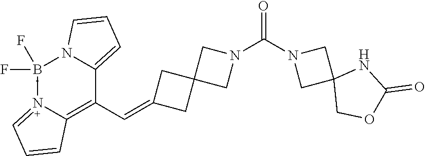

### Step a1) 2-(6-((4,4,5,5-tetramethyl-1,3,2-dioxaborolan-2-yl)methylene)-2-azaspiro[3.3]heptane-2-carbonyl)-7-oxa-2,5-diazaspiro[3.4]octan-6-one

In analogy to the procedures described in example 15 al) tert-butyl 6-((4,4,5,5-tetramethyl-1,3,2-dioxaborolan-2-yl)methylene)-2-azaspiro[3.3]heptane-2-carboxylate and 7-oxa-2,5-diazaspiro[3.4]octan-6-one (CAS: 1780174-72-2) were condensed to give the title compound as a colorless amorphous solid. LC-MS (ESI): m/z=390.2 [M+H]⁺.

### Step f) ((2-(6-oxo-7-oxa-2,5-diazaspiro[3.4]octane-2-carbonyl)-2-azaspiro[3.3]heptan-6-ylidene)methyl)boronic acid

The vinyl boronate was hydrolyzed to the respective boronic acid in analogy to the procedures described in example 15 f) to give the title compound as colorless solid. LC-MS (ESI): m/z=308.0 [M+H]⁺

### Step i) 2-(6-((5,5-difluoro-5H-4λ⁴,5λ⁴-dipyrrolo[1,2-c:2′,1′-f][1,3,2]diazaborinin-10-yl)methylene)-2-azaspiro[3.3]heptane-2-carbonyl)-7-oxa-2,5-diazaspiro[3.4]octan-6-one

In analogy to the procedures described in example 15 i) ((1-((4aR,8aS)-3-oxooctahydro-2H-pyrido[4,3-b][1,4]oxazine-6-carbonyl)piperidin-4-ylidene)methyl)boronic acid and [2-[(methylthio)(2H-pyrrol-2-ylidene)methyl]-1H-pyrrole](difluoroborane) (CAS: 892505-41-8) were coupled to give the title compound as an intensely red colored solid. LC-MS (ESI): m/z=461.2 [M−F]⁺

### Example 23 2-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)-7-oxa-2,5-diazaspiro[3.4]octan-6-one

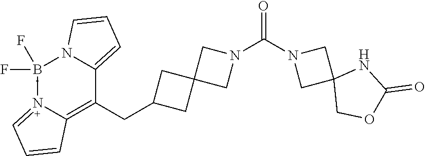

### Step j)

Example 22 was transformed to example 23 by the reduction of the olefinic double bond in analogy to the procedure described in example 16 j) to give a orange solid with green fluorescence in solution. LC-MS (ESI): m/z=436.1 [M−F]⁺.

### Example 24 6-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)-2H-benzo[b][1,4]oxazin-3(4H)-one

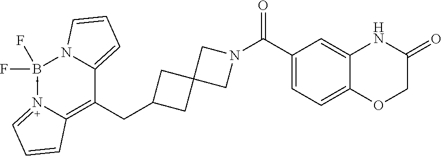

### Step a2) 6-(6-((4,4,5,5-tetramethyl-1,3,2-dioxaborolan-2-yl)methylene)-2-azaspiro[3.3]heptane-2-carbonyl)-2H-benzo[b][1,4]oxazin-3(4H)-one

tert-butyl 6-((4,4,5,5-tetramethyl-1,3,2-dioxaborolan-2-yl)methylene)-2-azaspiro[3.3]heptane-2-carboxylate (33.5 mg, 0.1 mmol) was boc deprotected by stirring in 5 ml TFA:DCM (1:4) at r.t. for 3 h. To remove traces of TFA it was twice coevaporated with 5 mL of toluene under reduced pressure. 3-oxo-3,4-dihydro-2H-benzo[b][1,4]oxazine-6-carboxylic acid (29.0 mg, 0.15 mmol)(CAS: 134997-87-8), HATU (57.0 mg, 0.15 mmol) and triethylamine were dissolved in anhydrous DCM (1 mL) and stirred at r.t. for 30 minutes. The deprotected amine TFA salt, dissolved in 0.5 mL DCM was added and the mixture was stirred at r.t. for 2 h. The mixture was concentrated under reduced pressure and purified via RP-HPLC (18 to 85% ACN:H₂O+0.1% TFA) to give the title compound as colorless solid (19 mg, 46%) LC-MS (ESI): m/z=411.2 [M+H]⁺.

### Step f) ((2-(3-oxo-3,4-dihydro-2H-benzo[b][1,4]oxazine-6-carbonyl)-2-azaspiro[3.3]heptan-6-ylidene)methyl)boronic acid

The vinyl boronate was hydrolyzed to the respective boronic acid in analogy to the procedures described in example 15 f) to give the title compound as colorless solid. LC-MS (ESI): m/z=329.2 [M+H]⁺

### Step i) 6-(6-((5,5-difluoro-5H-4λ⁴,5λ⁴-dipyrrolo[1,2-c:2′,1′-f][1,3,2]diazaborinin-10-yl)methylene)-2-azaspiro[3.3]heptane-2-carbonyl)-2H-benzo[b][1,4]oxazin-3(4H)-one

In analogy to the procedures described in example 15 i) ((1-((4aR,8aS)-3-oxooctahydro-2H-pyrido[4,3-b][1,4]oxazine-6-carbonyl)piperidin-4-ylidene)methyl)boronic acid and [2-[(methylthio)(2H-pyrrol-2-ylidene)methyl]-1H-pyrrole](difluoroborane) (CAS: 892505-41-8) were coupled to give the title compound as an intensely red colored solid. LC-MS (ESI): m/z=455.1 [M−F]

### Step j) 6-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)-2H-benzo[b][1,4]oxazin-3(4H)-one

In analogy to the procedure described in example 16 j) to give a orange solid with green fluorescence in solution. LC-MS (ESI): m/z=457.1 [M−F]f.

### Example 25 (6-(3-cyclopropyl-1H-1,2,4-triazol-1-yl)-2-azaspiro[3.3]heptan-2-yl)(6-((5,5-difluoro-5H-4λ⁴,5λ⁴-dipyrrolo[1,2-c:2,1′-f][1,3,2]diazaborinin-10-yl)methyl)-2-azaspiro[3.3]heptan-2-yl)methanone

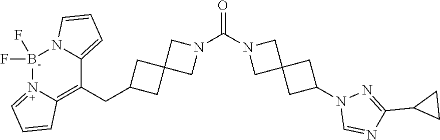

Synthesis of building block 6-(3-cyclopropyl-1H-1,2,4-triazol-1-yl)-2-azaspiro[3.3]heptane 2,2,2-trifluoroacetate (3 steps):

### Step 1) tert-butyl 6-methylsulfonyloxy-2-azaspiro[3.3]heptane-2-carboxylate

To a solution of tert-butyl 6-hydroxy-2-azaspiro[3.3]heptane-2-carboxylate (CAS: 1147557-97-8) (10.0 g, 46.9 mmol) in DCM (200 mL) was added TEA (7.12 g, 70.3 mmol, 9.79 mL) and MsCl (6.90 g, 60.2 mmol, 4.66 mL) dropwise at 0° C., The mixture was stirred at 30° C. for 2 h. The reaction mixture was quenched by addition of aq NaHCO 3 solution (200 mL), and then extracted with DCM (300 mL×2). The combined organic layers were dried over Na 2 SO 4, filtered and concentrated under reduced pressure to give a residue (13.5 g crude, 46.3 mmol, 98.8% yield), which was used into the next step without further purification. MS (ESI): m/z=236.2 [M+H]⁺

### Step 2) tert-butyl 6-(3-cyclopropyl-1,2,4-triazol-1-yl)-2-azaspiro[3.3]heptane-2-carboxylate

To a solution of tert-butyl 6-methylsulfonyloxy-2-azaspiro[3.3]heptane-2-carboxylate (12.0 g, 41.2 mmol, 90.0% purity) in ACN (200 mL) was added 3-cyclopropyl-1H-1,2,4-triazole (CAS: 1211390-33-8) (4.50 g, 41.2 mmol) and Cs 2 CO 3 (26.8 g, 82.4 mmol) at 25° C. The mixture was stirred at 100° C. for 16 h. The reaction mixture was filtered and concentrated under reduced pressure to give a residue. The residue was further separated by SFC to obtain the title compound (6.77 g, 22.2 mmol, 54.0% yield) as a brown solid. MS (ESI): m/z=305.2 [M+H]⁺

### Step 3) 6-(3-cyclopropyl-1H-1,2,4-triazol-1-yl)-2-azaspiro[3.3]heptane 2,2,2-trifluoroacetate

To a solution of tert-butyl 6-(3-cyclopropyl-1,2,4-triazol-1-yl)-2-azaspiro[3.3]heptane-2-carboxylate (6.00 g, 19.7 mmol) in DCM (120 mL) was added TFA (46.2 g, 405 mmol, 30 mL) at 25° C. The mixture was stirred at 30° C. for 16 h. The reaction mixture was filtered and concentrated under reduced pressure to give a residue. The crude product was used into the next step without further purification. The title compound (14.0 g, crude) was used in the next step without further purification. MS (ESI): m/z=205.2 [M+H]⁺

### Step a1) (6-(3-cyclopropyl-1H-1,2,4-triazol-1-yl)-2-azaspiro[3.3]heptan-2-yl)(6-((4,4,5,5-tetramethyl-1,3,2-dioxaborolan-2-yl)methylene)-2-azaspiro[3.3]heptan-2-yl)methanone

In analogy to the procedures described in example 15 al) tert-butyl 6-((4,4,5,5-tetramethyl-1,3,2-dioxaborolan-2-yl)methylene)-2-azaspiro[3.3]heptane-2-carboxylate and 6-(3-cyclopropyl-1H-1,2,4-triazol-1-yl)-2-azaspiro[3.3]heptane 2,2,2-trifluoroacetate were condensed to give the title compound as a colorless amorphous solid. LC-MS (ESI): m/z=466.3 [M+H]⁺.

### Step f) ((2-(6-(3-cyclopropyl-1H-1,2,4-triazol-1-yl)-2-azaspiro[3.3]heptane-2-carbonyl)-2-azaspiro[3.3]heptan-6-ylidene)methyl)boronic acid

The vinyl boronate was hydrolyzed to the respective boronic acid in analogy to the procedures described in example 15 f) to give the title compound as colorless solid. LC-MS (ESI): m/z=329.1 [M+H]⁺

### Step i) (6-(3-cyclopropyl-1H-1,2,4-triazol-1-yl)-2-azaspiro[3.3]heptan-2-yl)(6-((5,5-difluoro-5H-4λ⁴,5λ⁴-dipyrrolo[1,2-c:2′,1′-f][1,3,2]diazaborinin-10-yl)methylene)-2-azaspiro[3.3]heptan-2-yl)methanone

In analogy to the procedures described in example 15 i) ((2-(6-(3-cyclopropyl-1H-1,2,4-triazol-1-yl)-2-azaspiro[3.3]heptane-2-carbonyl)-2-azaspiro[3.3]heptan-6-ylidene)methyl)boronic acid and [2-[(methylthio)(2H-pyrrol-2-ylidene)methyl]-1H-pyrrole](difluoroborane) (CAS: 892505-41-8) were coupled to give the title compound as an intensely red colored solid. LC-MS (ESI): m/z=455.1 [M−F]⁺

### Step j) (6-(3-cyclopropyl-1H-1,2,4-triazol-1-yl)-2-azaspiro[3.3]heptan-2-yl)(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)methanone

In analogy to the procedure described in example 16 j) to give a orange solid with green fluorescence in solution. LC-MS (ESI): m/z=457.1 [M−F]f.

### Example 26 (6-((5,5-difluoro-5H-424, 5λ⁴-dipyrrolo[1,2-c:2′,1′-f][1,3,2]diazaborinin-10-yl)methyl)-2-azaspiro[3.3]heptan-2-yl)(4-(5-methyloxazolo[4,5-b]pyridin-2-yl)piperazin-1-yl)methanone

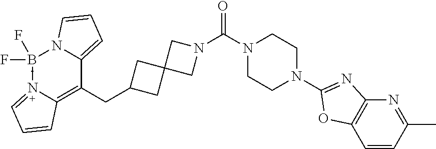

### Step a1) (4-(5-methyloxazolo[4,5-b]pyridin-2-yl)piperazin-1-yl)(6-((4,4,5,5-tetramethyl-1,3,2-dioxaborolan-2-yl)methylene)-2-azaspiro[3.3]heptan-2-yl)methanone

In analogy to the procedures described in example 15 al) tert-butyl 6-((4,4,5,5-tetramethyl-1,3,2-dioxaborolan-2-yl)methylene)-2-azaspiro[3.3]heptane-2-carboxylate and 5-methyl-2-(piperazin-1-yl)oxazolo[4,5-b]pyridine (CAS: 1035840-99-3) were condensed to give the title compound as a colorless amorphous solid. LC-MS (ESI): m/z=480.2 [M+H]⁺.

### Step f) ((2-(3-oxo-3,4-dihydro-2H-benzo[b][1,4]oxazine-6-carbonyl)-2-azaspiro[3.3]heptan-6-ylidene)methyl)boronic acid

The vinyl boronate was hydrolyzed to the respective boronic acid in analogy to the procedures described in example 15 f) to give the title compound as colorless solid. LC-MS (ESI): m/z=398.2 [M+H]⁺

### Step i) (6-((5,5-difluoro-5H-4λ⁴,5λ⁴-dipyrrolo[1,2-c:2′,1′-f][1,3,2]diazaborinin-10-yl)methylene)-2-azaspiro[3.3]heptan-2-yl)(4-(5-methyloxazolo[4,5-b]pyridin-2-yl)piperazin-1-yl)methanone

In analogy to the procedures described in example 15 i) ((1-((4aR,8aS)-3-oxooctahydro-2H-pyrido[4,3-b][1,4]oxazine-6-carbonyl)piperidin-4-ylidene)methyl)boronic acid and [2-[(methylthio)(2H-pyrrol-2-ylidene)methyl]-1H-pyrrole](difluoroborane) (CAS: 892505-41-8) were coupled to give the title compound as an intensely red colored solid. LC-MS (ESI): m/z=524.2 [M−F]⁺

### Step j) (6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)(4-(5-methyloxazolo[4,5-b]pyridin-2-yl)piperazin-1-yl)methanone

In analogy to the procedure described in example 16 j) to give a orange solid with green fluorescence in solution. LC-MS (ESI): m/z=526.2 [M−F]f.

### Example 27 (S)-1-(6-((5,5-difluoro-5H-4λ⁴,5λ⁴-dipyrrolo[1,2-c:2,1′-f][1,3,2]diazaborinin-10-yl)methyl)-2-azaspiro[3.3]heptane-2-carbonyl)pyrrolidine-3-carboxamide

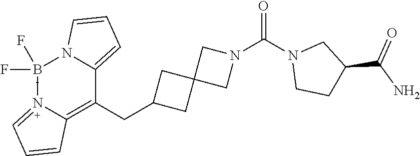

### Step a1) (S)-1-(6-((4,4,5,5-tetramethyl-1,3,2-dioxaborolan-2-yl)methylene)-2-azaspiro[3.3]heptane-2-carbonyl)pyrrolidine-3-carboxamide

In analogy to the procedures described in example 15 al) tert-butyl 6-((4,4,5,5-tetramethyl-1,3,2-dioxaborolan-2-yl)methylene)-2-azaspiro[3.3]heptane-2-carboxylate and (S)-pyrrolidine-3-carboxamide hydrochloride (CAS: 1279048-81-5) were condensed to give the title compound as a colorless amorphous solid. LC-MS (ESI): m/z=376.2 [M+H]⁺.

### Step f) (S)-((2-(3-carbamoylpyrrolidine-1-carbonyl)-2-azaspiro[3.3]heptan-6-ylidene)methyl)boronic acid

The vinyl boronate was hydrolyzed to the respective boronic acid in analogy to the procedures described in example 15 f) to give the title compound as colorless solid. LC-MS (ESI): m/z=294.1 [M+H]⁺

### Step i) (S)-1-(6-((5,5-difluoro-5H-4λ⁴,5λ⁴-dipyrrolo[1,2-c:2′,1′-f][1,3,2]diazaborinin-10-yl)methylene)-2-azaspiro[3.3]heptane-2-carbonyl)pyrrolidine-3-carboxamide

In analogy to the procedures described in example 15 i) ((1-((4aR,8aS)-3-oxooctahydro-2H-pyrido[4,3-b][1,4]oxazine-6-carbonyl)piperidin-4-ylidene)methyl)boronic acid and [2-[(methylthio)(2H-pyrrol-2-ylidene)methyl]-1H-pyrrole](difluoroborane) (CAS: 892505-41-8) were coupled to give the title compound as an intensely red colored solid. LC-MS (ESI): m/z=420.2 [M−F]⁺

### Step j) (S)-1-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)pyrrolidine-3-carboxamide

In analogy to the procedure described in example 16 j) to give a orange solid with green fluorescence in solution. LC-MS (ESI): m/z=442.1 [M+H]⁺.

### Example 28 (R)-4-(3-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)-3-oxopropyl)oxazolidin-2-one

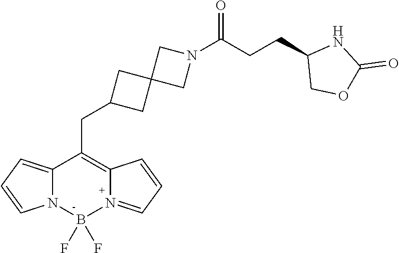

### Synthesis of the building block: 3-[(4R)-2-Oxooxazolidin-4-yl]propanoic acid (3 steps)

### Step 1) Methyl (S)-4-((tert-butoxycarbonyl)amino)-5-hydroxypentanoate

To a solution of (R)-2-((tert-butoxycarbonyl)amino)-5-methoxy-5-oxopentanoic acid (1 g, 3.83 mmol, CAS: 76379-01-6) in THE (15 mL) at −10° C. was added N-methylmorpholine (421 μL, 3.83 mmol), followed by ethyl chloroformate (368 μL, 3.83 mmol) and the reaction mixture was stirred at this temperature for 10 minutes. Addition of NaBH₄ (434 mg, 11.5 mmol) in one portion did not cause a temperature increase. MeOH (35 mL) was added dropwise between −1° C. and 17° C. over 30 min. Stirring was continued in an ice bath for 1 h. A 1 M aqueous KHSO₄ sol. (40 mL) was added dropwise to the reaction mixture and then the organic solvents were evaporated. The aqueous layer was extracted twice with ethyl acetate. The combined organic layers were washed with aqueous 1 M KHSO₄ solution and sat. aqueous NaHCO₃ solution, dried over MgSO₄, filtered, treated with silica gel and evaporated. The crude compound was purified by silica gel chromatography using a MPLC system eluting with a gradient of n-heptane:ethyl acetate (100:0 to 30:70) to get the title compound as a colorless oil (0.70 g, 66%). MS (ESI): m/z=192.1 [M+H]⁺.

### Step 2) Methyl (R)-3-(2-oxooxazolidin-4-yl)propanoate

To a solution of methyl (S)-4-((tert-butoxycarbonyl)amino)-5-hydroxypentanoate (690 mg, 2.79 mmol, 1.0 equiv) in THE (8.8 mL) was added dropwise thionyl chloride (611 μL, 8.37 mmol, 3.0 equiv) and the solution was stirred at RT for 3 h. Silica gel was added and the reaction mixture was evaporated. The compound was purified by silica gel chromatography using a MPLC system eluting with a gradient of n-heptane:ethyl acetate (100:0 to 0:100) to afford the title compound as a colorless oil (404 mg, 79%). MS (ESI): m/z=174.1 [M+H]⁺.

### Step 3) (R)-3-(2-Oxooxazolidin-4-yl)propanoic acid

To a solution of methyl (S)-3-(2-oxooxazolidin-4-yl)propanoate (400 mg, 2.31 mmol, 1.0 equiv) in 1,4-dioxane (2 mL) and water (2 mL) was added lithium hydroxide monohydrate (107 mg, 2.54 mmol, 1.1 equiv) and the reaction mixture was stirred at RT for 2 h. 1,4-Dioxane was evaporated and aqueous HCl (2.54 mL, 2.54 mmol, 1.1 equiv) was added dropwise to the solution. The aqueous layer was extracted five times with ethyl acetate. The combined organic layers were dried over MgSO 4, filtered and evaporated to get the title compound as a colorless solid (330 mg, 86%). MS (ESI): m/z=160.1 [M+H]⁺.

### Step a2) (R)-4-(3-oxo-3-(6-((4,4,5,5-tetramethyl-1,3,2-dioxaborolan-2-yl)methylene)-2-azaspiro[3.3]heptan-2-yl)propyl)oxazolidin-2-one

In analogy to the procedures described in example 25 a2) tert-butyl 6-((4,4,5,5-tetramethyl-1,3,2-dioxaborolan-2-yl)methylene)-2-azaspiro[3.3]heptane-2-carboxylate and 3-[(4R)-2-oxooxazolidin-4-yl]propanoic acid were condensed to give the title compound as a colorless amorphous solid. LC-MS (ESI): m/z=372.2 [M+H]⁺.

### Step f) (R)-((2-(3-(2-oxooxazolidin-4-yl)propanoyl)-2-azaspiro[3.3]heptan-6-ylidene)methyl)boronic acid

The vinyl boronate was hydrolyzed to the respective boronic acid in analogy to the procedures described in example 15 f) to give the title compound as colorless solid. LC-MS (ESI): m/z=295.1 [M+H]⁺

### Step i) (R)-4-(3-(6-((5,5-difluoro-5H-4λ⁴,5λ⁴-dipyrrolo[1,2-c:2′,1′-f][1,3,2]diazaborinin-10-yl)methylene)-2-azaspiro[3.3]heptan-2-yl)-3-oxopropyl)oxazolidin-2-one

In analogy to the procedures described in example 15 i) ((1-((4aR,8aS)-3-oxooctahydro-2H-pyrido[4,3-b][1,4]oxazine-6-carbonyl)piperidin-4-ylidene)methyl)boronic acid and [2-[(methylthio)(2H-pyrrol-2-ylidene)methyl]-1H-pyrrole](difluoroborane) (CAS: 892505-41-8) were coupled to give the title compound as an intensely red colored solid. LC-MS (ESI): m/z=421.2 [M−F]⁺

### Step j) (R)-4-(3-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)-3-oxopropyl)oxazolidin-2-one

In analogy to the procedure described in example 16 j) to give an orange solid with green fluorescence in solution. LC-MS (ESI): m/z=423.2 [M−F]⁺.

### Example 29 (4aR,8aS)-6-(6-((5,5-difluoro-3-phenyl-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one

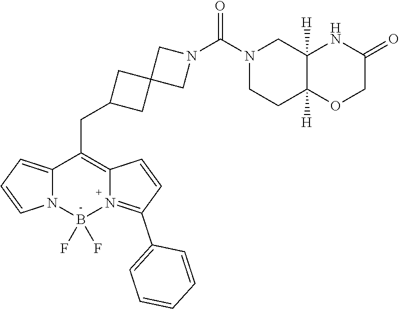

### Step g) 5,5-difluoro-10-(methylthio)-3-phenyl-5H-514,614-dipyrrolo[1,2-c:2′,1′-f][1,3,2]diazaborinine

An oven-dried flask equipped with a stir bar was charged with [2-[(methylthio)(2H-pyrrol-2-ylidene)methyl]-1H-pyrrole](difluoroborane) (71.4 mg, 0.3 mmol) (CAS: 892505-41-8), aniline (55.9 mg, 0.6 mmol) (CAS: 62-53-3) and anhydrous ACN. The mixture was stirred until the solids dissolved. tert-Butyl nitrite (107 μL, 0.9 mmol) (CAS: 540-80-7) was then added with a syringe. A fine bubbling was observed thereafter. The reaction was heated to 40° C. for 18 h. Excess of solvent was removed under vacuum and the crude material was dissolved ACN:H₂O (4:1) and purified via RP-HPLC (25 to 95% ACN:H₂O+0.1% TFA) to give the title compound as a dark red solid(18.6 mg, 30%). LC-MS (ESI): m/z=295.0 [M−F]⁺.

### Step i) (4aR,8aS)-6-(6-((5,5-difluoro-3-phenyl-5H-4λ⁴,5λ⁴-dipyrrolo[1,2-c:2′,1′-f][1,3,2]diazaborinin-10-yl)methylene)-2-azaspiro[3.3]heptane-2-carbonyl)hexahydro-2H-pyrido[4,3-b][1,4]oxazin-3(4H)-one

In analogy to the procedures described in example 15 i) ((2-((4aR,8aS)-3-oxooctahydro-2H-pyrido[4,3-b][1,4]oxazine-6-carbonyl)-2-azaspiro[3.3]heptan-6-ylidene)methyl)boronic acid and 5,5-difluoro-10-(methylthio)-3-phenyl-5H-514,614-dipyrrolo[1,2-c:2′,1′-f][1,3,2]diazaborinine were coupled to give the title compound as an intensely red colored solid. LC-MS (ESI): m/z=538.2 [M−F]⁺.

### Step j) (4aR,8aS)-6-(6-((5,5-difluoro-3-phenyl-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one

In analogy to the procedure described in example 16 j) to give a red solid with orange fluorescence in solution. LC-MS (ESI): m/z=540.2 [M−F]⁺.

### Example 30 (4aR,8aS)-6-(6-((5,5-difluoro-3-(1H-pyrrol-2-yl)-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one

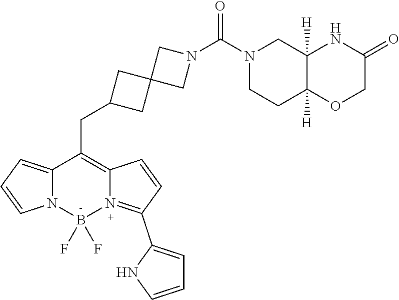

### Step g) 5,5-difluoro-10-(methylthio)-3-(1H-pyrrol-2-yl)-5H-5λ⁴,6λ⁴-dipyrrolo[1,2-c:2′,1′-f][1,3,2]diazaborinine

A microwave vial with a stir bar was charged with [2-[(methylthio)(2H-pyrrol-2-ylidene)methyl]-1H-pyrrole](difluoroborane) (71.4 mg, 0.3 mmol) (CAS: 892505-41-8) and 1H-pyrrole (1.04 mL, 15 mmol) (CAS: 109-97-7). The vial was sealed and heated to 150° C. via microwave irradiation for 3 h. Excess 1H-pyrrol was evaporated under reduced pressure and the residue was purified via RP-HPLC (25 to 95% ACN:H₂O+0.1% TFA) to give the title compound as a dark purple solid (8.0 mg, 9%). LC-MS (ESI): m/z=304.1 [M+H]⁺.

### Step i) (4aR,8aS)-6-(6-((5,5-difluoro-3-(1H-pyrrol-2-yl)-5H-4λ⁴,5λ⁴-dipyrrolo[1,2-c:2′,1′-f][1,3,2]diazaborinin-10-yl)methylene)-2-azaspiro[3.3]heptane-2-carbonyl)hexahydro-2H-pyrido[4,3-b][1,4]oxazin-3(4H)-one

In analogy to the procedures described in example 15 i) ((2-((4aR,8aS)-3-oxooctahydro-2H-pyrido[4,3-b][1,4]oxazine-6-carbonyl)-2-azaspiro[3.3]heptan-6-ylidene)methyl)boronic acid and 5,5-difluoro-10-(methylthio)-3-(1H-pyrrol-2-yl)-5H-5λ⁴,6λ⁴-dipyrrolo[1,2-c:2′,1′-f][1,3,2]diazaborinine were coupled to give the title compound as an intensely red colored solid. LC-MS (ESI): m/z=527.2 [M−F]⁺.

### Step j) (4aR,8aS)-6-(6-((5,5-difluoro-3-(1H-pyrrol-2-yl)-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one

In analogy to the procedure described in example 16 j) to give a purple solid with red fluorescence in solution. LC-MS (ESI): m/z=529.3 [M−F]⁺.

### Example 31 (6-(3-cyclopropyl-1H-1,2,4-triazol-1-yl)-2-azaspiro[3.3]heptan-2-yl)(6-((5,5-difluoro-3-(1H-pyrrol-2-yl)-5H-5λ⁴,6λ⁴-dipyrrolo[1,2-c:2′,1′-f][1,3,2]diazaborinin-10-yl)methyl)-2-azaspiro[3.3]heptan-2-yl)methanone

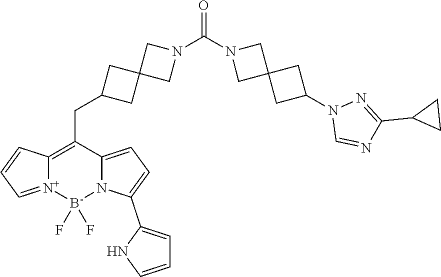 [SMILES: O=C(N1CC2(CC(CC3=C4C=CC=[N+]4[B-](F)(F)n4c3ccc4-c3ccc[nH]3)C2)C1)N1CC2(CC(n3cnc(C4CC4)n3)C2)C1]

### Step i) (6-(3-cyclopropyl-1H-1,2,4-triazol-1-yl)-2-azaspiro[3.3]heptan-2-yl)(6-((5,5-difluoro-3-(1H-pyrrol-2-yl)-5H-5λ⁴,6λ⁴-dipyrrolo[1,2-c:2′,1′-f][1,3,2]diazaborinin-10-yl)methylene)-2-azaspiro[3.3]heptan-2-yl)methanone

In analogy to the procedures described in example 15 i) ((2-(6-(3-cyclopropyl-1H-1,2,4-triazol-1-yl)-2-azaspiro[3.3]heptane-2-carbonyl)-2-azaspiro[3.3]heptan-6-ylidene)methyl)boronic acid and 5,5-difluoro-10-(methylthio)-3-(1H-pyrrol-2-yl)-5H-5λ⁴,6λ⁴-dipyrrolo[1,2-c:2′,1′-f][1,3,2]diazaborinine were coupled to give the title compound as an intensely purple colored solid. LC-MS (ESI): m/z=595.2 [M+H]⁺.

### Step j) (6-(3-cyclopropyl-1H-1,2,4-triazol-1-yl)-2-azaspiro[3.3]heptan-2-yl)(6-((5,5-difluoro-3-(1H-pyrrol-2-yl)-5H-5λ⁴,6λ⁴-dipyrrolo[1,2-c:2′,1′-f][1,3,2]diazaborinin-10-yl)methyl)-2-azaspiro[3.3]heptan-2-yl)methanone

The title compound was synthesized in analogy to the procedure described in example 16 j) from the intermediate above. The title compound was obtained as a purple solid with red fluorescence in solution. LC-MS (ESI): m/z=597.2 [M+H]⁺.

The following examples can be prepared in analogy to the examples described above:

### Example 32 (S)-(3-(1H-1,2,3-triazol-5-yl)pyrrolidin-1-yl)(6-((5,5-difluoro-5H-4λ⁴,5λ⁴-dipyrrolo[1,2-c:2,1′-f][1,3,2]diazaborinin-10-yl)methyl)-2-azaspiro[3.3]heptan-2-yl)methanone

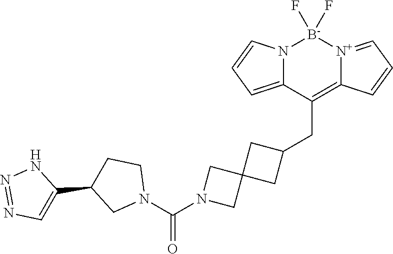 [SMILES: O=C(N1CC[C@H](c2cnn[nH]2)C1)N1CC2(CC(CC3=C4C=CC=[N+]4[B-](F)(F)n4cccc43)C2)C1]

### Example 33 (S)-(3-(1H-1,2,3-triazol-5-yl)pyrrolidin-1-yl)(7-((5,5-difluoro-5H-4λ⁴,5λ⁴-dipyrrolo[1,2-c:2,1′-f][1,3,2]diazaborinin-10-yl)methyl)-2-azaspiro[3.5]nonan-2-yl)methanone

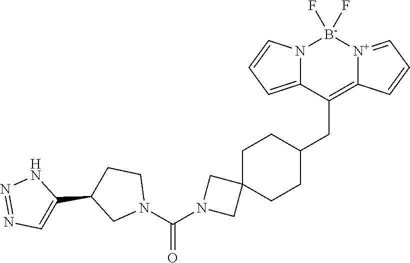 [SMILES: O=C(N1CC[C@H](c2cnn[nH]2)C1)N1CC2(CCC(CC3=C4C=CC=[N+]4[B-](F)(F)n4cccc43)CC2)C1]

### Example 34 (6-(3-cyclopropyl-1H-1,2,4-triazol-1-yl)-2-azaspiro[3.3]heptan-2-yl)(7-((5,5-difluoro-5H-4λ⁴,5λ⁴-dipyrrolo[1,2-c:2,1′-f][1,3,2]diazaborinin-10-yl)methyl)-2-azaspiro[3.5]nonan-2-yl)methanone

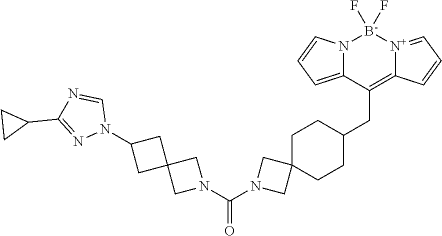 [SMILES: O=C(N1CC2(CCC(CC3=C4C=CC=[N+]4[B-](F)(F)n4cccc43)CC2)C1)N1CC2(CC(n3cnc(C4CC4)n3)C2)C1]

### Example 35 6-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonane-2-carbonyl)-2H-benzo[b][1,4]oxazin-3(4H)-one

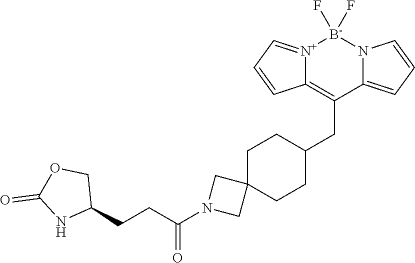 [SMILES: O=C1N[C@H](CCC(=O)N2CC3(CCC(CC4=C5C=CC=[N+]5[B-](F)(F)n5cccc54)CC3)C2)CO1]

### Example 36 (R)-4-(3-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonan-2-yl)-3-oxopropyl)oxazolidin-2-one

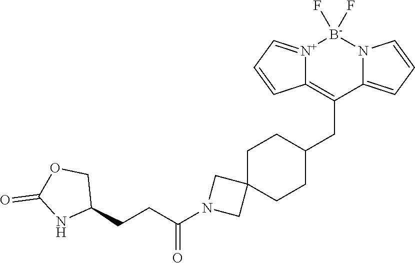 [SMILES: O=C1N[C@H](CCC(=O)N2CC3(CCC(CC4=C5C=CC=[N+]5[B-](F)(F)n5cccc54)CC3)C2)CO1]

### Example 37 (S)-1-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonane-2-carbonyl)pyrrolidine-3-carboxamide

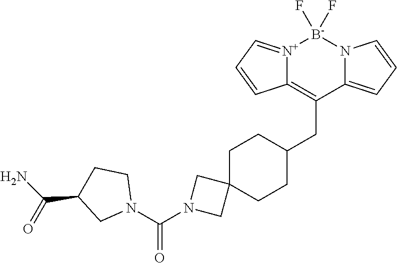 [SMILES: NC(=O)[C@H]1CCN(C(=O)N2CC3(CCC(CC4=C5C=CC=[N+]5[B-](F)(F)n5cccc54)CC3)C2)C1]

### Example 38 (7-((5,5-difluoro-5H-42, 5λ⁴-dipyrrolo[1,2-c:2′,1′-f][1,3,2]diazaborinin-10-yl)methyl)-2-azaspiro[3.5]nonan-2-yl)(4-(5-methyloxazolo[4,5-b]pyridin-2-yl)piperazin-1-yl)methanone

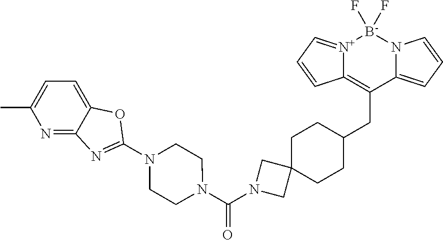 [SMILES: Cc1ccc2oc(N3CCN(C(=O)N4CC5(CCC(CC6=C7C=CC=[N+]7[B-](F)(F)n7cccc76)CC5)C4)CC3)nc2n1]

### Example 39 (4aR,8aS)-6-(2-((5,5-difluoro-5H-4λ⁴,5λ⁴-dipyrrolo[1,2-c:2′,1′-f][1,3,2]diazaborinin-10-yl)methyl)-7-azaspiro[3.5]nonane-7-carbonyl)hexahydro-2H-pyrido[4,3-b][1,4]oxazin-3(4H)-one

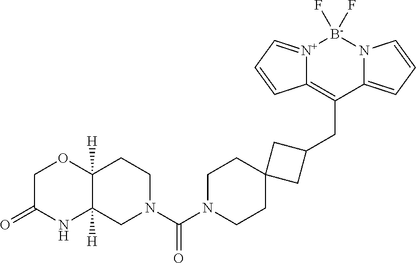 [SMILES: O=C1CO[C@H]2CCN(C(=O)N3CCC4(CC3)CC(CC3=C5C=CC=[N+]5[B-](F)(F)n5cccc53)C4)C[C@H]2N1]

### Example 40 2-(2-((5,5-difluoro-5H-4λ⁴,5λ⁴-dipyrrolo[1,2-c:2′,1′-f][1,3,2]diazaborinin-10-yl)methyl)-7-azaspiro[3.5]nonane-7-carbonyl)-7-oxa-2,5-diazaspiro[3.4]octan-6-one

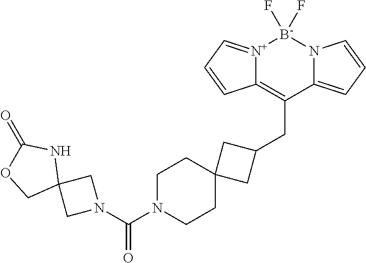 [SMILES: O=C1NC2(CO1)CN(C(=O)N1CCC3(CC1)CC(CC1=C4C=CC=[N+]4[B-](F)(F)n4cccc41)C3)C2]

### Example 41 (S)-(3-(1H-1,2,3-triazol-5-yl)pyrrolidin-1-yl)(2-((5,5-difluoro-5H-4λ⁴,5λ⁴-dipyrrolo[1,2-c:2,1′-f][1,3,2]diazaborinin-10-yl)methyl)-7-azaspiro[3.5]nonan-7-yl)methanone

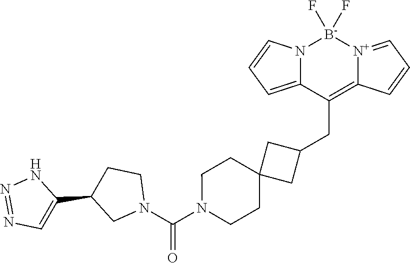 [SMILES: O=C(N1CCC2(CC1)CC(CC1=C3C=CC=[N+]3[B-](F)(F)n3cccc31)C2)N1CC[C@H](c2cnn[nH]2)C1]

### Example 42 (6-(3-cyclopropyl-1H-1,2,4-triazol-1-yl)-2-azaspiro[3.3]heptan-2-yl)(2-((5,5-difluoro-5H-4λ⁴,5λ⁴-dipyrrolo[1,2-c:2,1′-f][1,3,2]diazaborinin-10-yl)methyl)-7-azaspiro[3.5]nonan-7-yl)methanone

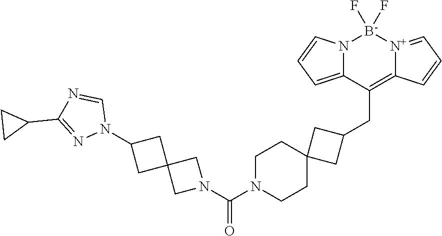 [SMILES: O=C(N1CCC2(CC1)CC(CC1=C3C=CC=[N+]3[B-](F)(F)n3cccc31)C2)N1CC2(CC(n3cnc(C4CC4)n3)C2)C1]

### Example 43 6-(2-((5,5-difluoro-5H-4λ⁴,5λ⁴-dipyrrolo[1,2-c:2′,1′-f][1,3,2]diazaborinin-10-yl)methyl)-7-azaspiro[3.5]nonane-7-carbonyl)-2H-benzo[b][1,4]oxazin-3(4H)-one

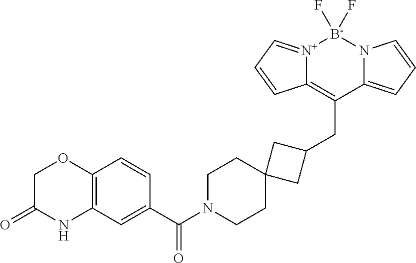 [SMILES: O=C1COc2ccc(C(=O)N3CCC4(CC3)CC(CC3=C5C=CC=[N+]5[B-](F)(F)n5cccc53)C4)cc2N1]

### Example 44 (R)-4-(3-(2-((5,5-difluoro-5H-4λ⁴,5λ⁴-dipyrrolo[1,2-c:2′,1′-f][1,3,2]diazaborinin-10-yl)methyl)-7-azaspiro[3.5]nonan-7-yl)-3-oxopropyl)oxazolidin-2-one

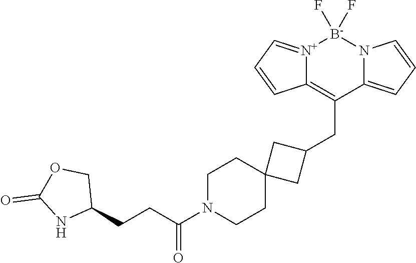 [SMILES: O=C1N[C@H](CCC(=O)N2CCC3(CC2)CC(CC2=C4C=CC=[N+]4[B-](F)(F)n4cccc42)C3)CO1]

### Example 45 (S)-1-(2-((5,5-difluoro-5H-4λ⁴,5λ⁴-dipyrrolo[1,2-c:2′,1′-f][1,3,2]diazaborinin-10-yl)methyl)-7-azaspiro[3.5]nonane-7-carbonyl)pyrrolidine-3-carboxamide

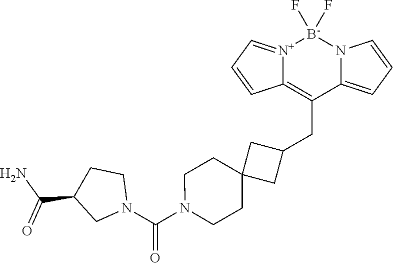 [SMILES: NC(=O)[C@H]1CCN(C(=O)N2CCC3(CC2)CC(CC2=C4C=CC=[N+]4[B-](F)(F)n4cccc42)C3)C1]

### Example 46 (2-((5,5-difluoro-5H-4λ⁴,5λ⁴-dipyrrolo[1,2-c:2′,1′-f][1,3,2]diazaborinin-10-yl)methyl)-7-azaspiro[3.5]nonan-7-yl)(4-(5-methyloxazolo[4,5-b]pyridin-2-yl)piperazin-1-yl)methanone

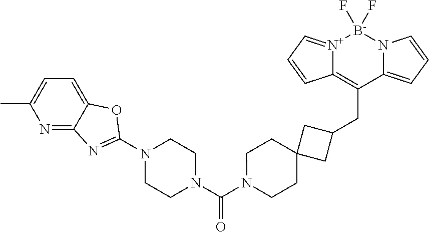

1. A compound of formula (I) 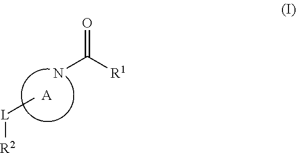
or a pharmaceutically acceptable salt thereof, wherein:
R¹ is selected from: 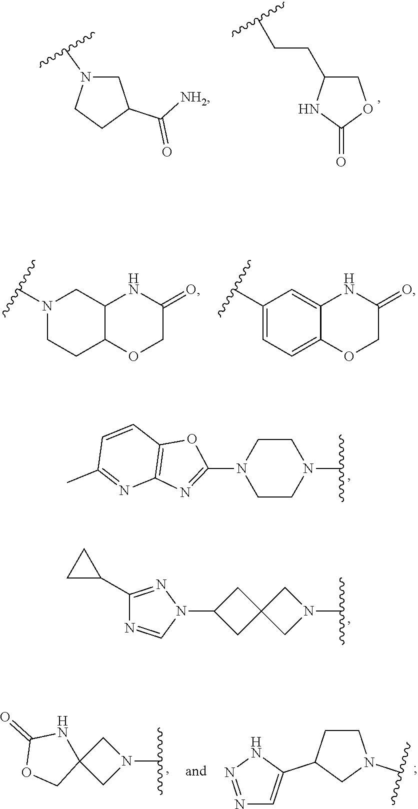
wherein a wavy line indicates the point of attachment to the carbonyl group of formula (I);
R² is selected from: 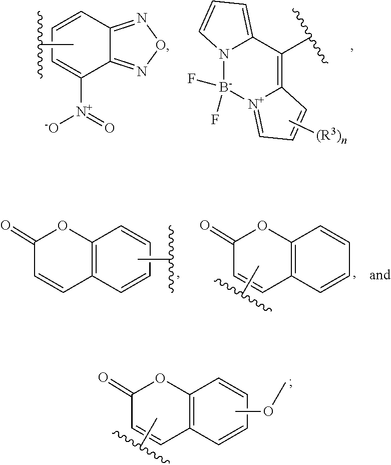
wherein a wavy line indicates the point of attachment to L;
each R³ is independently selected from hydrogen, halogen, C₁-C₆-alkyl, C₁-C₆-alkoxy, phenyl, 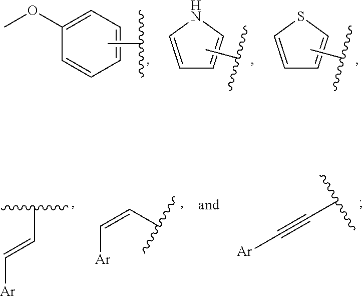
n is 1, 2 or 3;
Ar is C₆-C₁₀-aryl or 5- to 14-membered heteroaryl;
A is selected from:
(i) 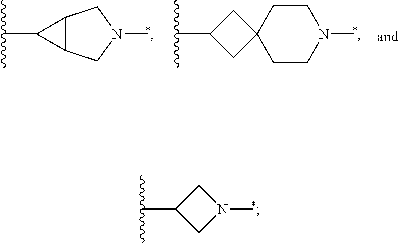
and
L is selected from a covalent bond, —CH₂—, —O—, —OCH₂—, —CH₂O— and —CH₂OCH₂—; and
(ii) 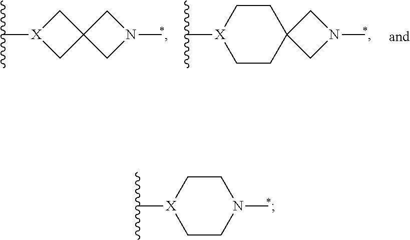
wherein
(i) X is N; and L is selected from a covalent bond, —CH₂—, and —OCH₂—; or
(ii) X is CH; and L is selected from a covalent bond, —CH₂—, —NH—, —NMe-, —O—, —OCH₂—, —CH₂O— and —CH₂OCH₂—; or
(iii) X and L, taken together, form a group 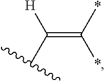
wherein the wavy line indicates the point of attachment of L to R², and the two asterisks indicate the points of attachment of X to the respective neighbouring atoms within ring A;
wherein a wavy line indicates the point of attachment to L; and
an asterisk indicates the point of attachment to the carbonyl group of formula (I).

2. The compound of formula (I) according to claim 1, or a pharmaceutically acceptable salt thereof, wherein R¹ is selected from 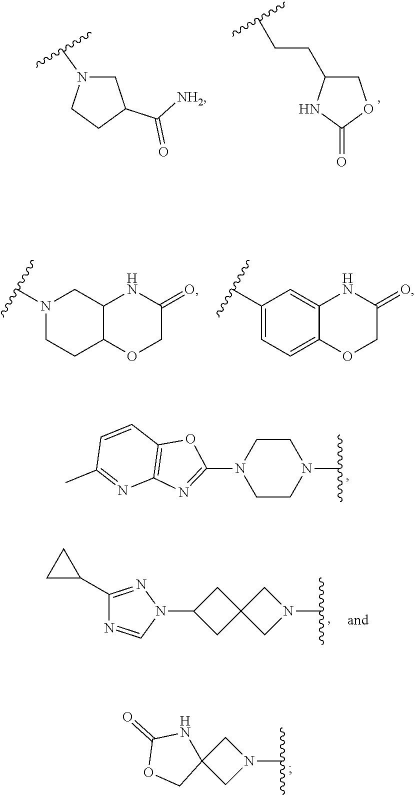
wherein a wavy line indicates the point of attachment to the carbonyl group of formula (I).

3. The compound of formula (I) according to claim 1, or a pharmaceutically acceptable salt thereof, wherein R¹ is selected from 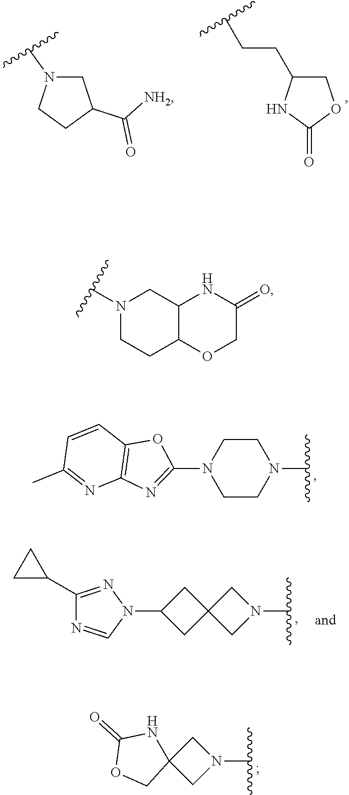 [SMILES: *N1CCC(C(N)=O)C1 ; *CCC1COC(=O)N1 ; *N1CCC2OCC(=O)NC2C1 ; *N1CCN(c2nc3nc(C)ccc3o2)CC1 ; *N1CC2(CC(n3cnc(C4CC4)n3)C2)C1 ; *N1CC2(COC(=O)N2)C1]
wherein a wavy line indicates the point of attachment to the carbonyl group of formula (I).

4. The compound of formula (I) according to claim 2, or a pharmaceutically acceptable salt thereof, wherein R¹ is selected from 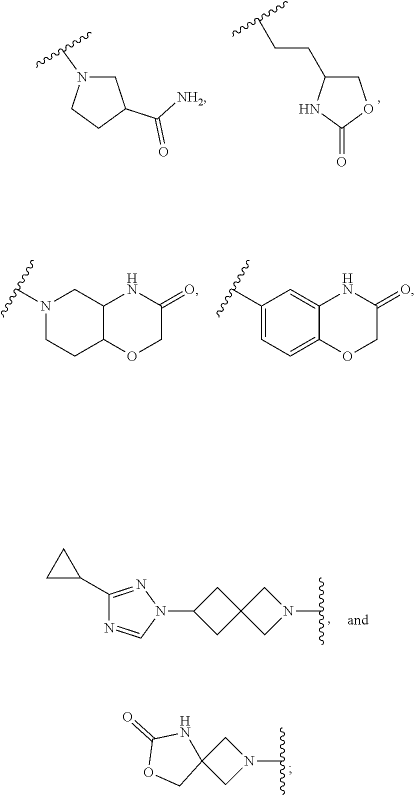
wherein a wavy line indicates the point of attachment to the carbonyl group of formula (I).

5. The compound of formula (I) according to claim 4, or a pharmaceutically acceptable salt thereof, wherein R¹ is selected from 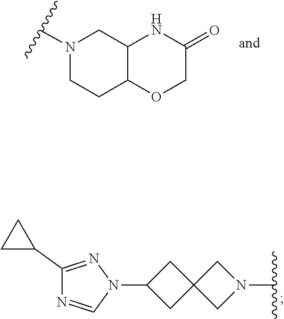 [SMILES: *N1CCC2OCC(=O)NC2C1 ; *N1CC2(CC(n3cnc(C4CC4)n3)C2)C1]
wherein a wavy line indicates the point of attachment to the carbonyl group of formula (I).

6. The compound of formula (I) according to any one of claims 1 to 5, or a pharmaceutically acceptable salt thereof, wherein:
R² is selected from: 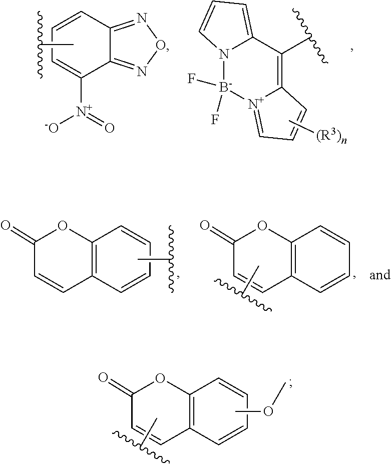
wherein a wavy line indicates the point of attachment to L;
R³ is selected from hydrogen, phenyl, and 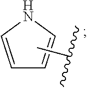
and
n is 1.

7. The compound of formula (I) according to claim 6, or a pharmaceutically acceptable salt thereof, wherein:
R² is selected from: 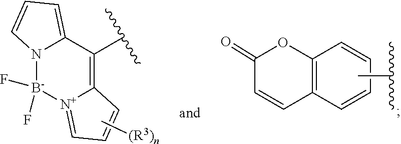
wherein a wavy line indicates the point of attachment to L;
R³ is selected from hydrogen and 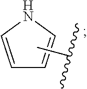
and
n is 1.

8. The compound of formula (I) according to claim 7, or a pharmaceutically acceptable salt thereof, wherein:
R² is: 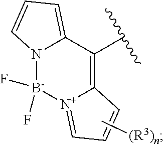
wherein a wavy line indicates the point of attachment to L;
R³ is hydrogen; and
n is 1.

9. The compound of formula (I) according to any one of claims 1 to 8, or a pharmaceutically acceptable salt thereof, wherein:
A is selected from:
(i) 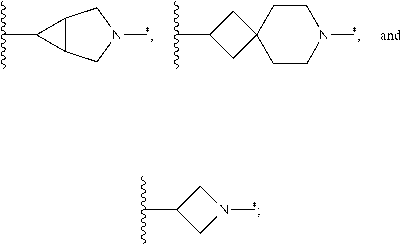
and
L is selected from a covalent bond, —O—, and —OCH₂—; and
(ii) 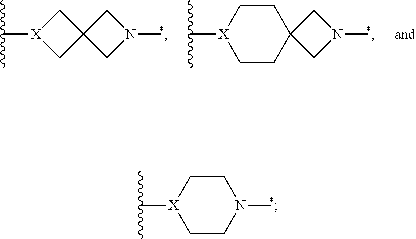
wherein
(i) X is N; and L is —CH₂—; or
(ii) X is CH; and L is selected from —CH₂—, —NH— and —O—; or
(iii) X and L, taken together, form a group 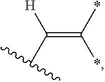
wherein the wavy line indicates the point of attachment of L to R², and the two asterisks indicate the points of attachment of X to the respective neighbouring atoms within ring A;
wherein a wavy line indicates the point of attachment to L; and
an asterisk indicates the point of attachment to the carbonyl group of formula (I).

10. The compound of formula (I) according to claim 9, or a pharmaceutically acceptable salt thereof, wherein:
A is selected from:
(i) 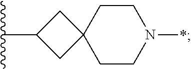
and
L is —O—; and
(ii) 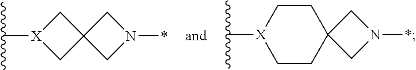
wherein
(i) X is CH; and L is selected from —CH₂— and —O—; or
(ii) X and L, taken together, form a group 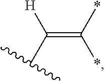
wherein the wavy line indicates the point of attachment of L to R², and the two asterisks indicate the points of attachment of X to the respective neighbouring atoms within ring A;
wherein a wavy line indicates the point of attachment to L; and
an asterisk indicates the point of attachment to the carbonyl group of formula (I).

11. The compound of formula (I) according to claim 10, or a pharmaceutically acceptable salt thereof, wherein:
A is 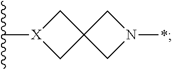
X is CH; and
L is —CH₂—;
wherein the wavy line indicates the point of attachment to L; and
the asterisk indicates the point of attachment to the carbonyl group of formula (I).

12. The compound of formula (I) according to claim 1, or a pharmaceutically acceptable salt thereof, wherein:
R¹ is selected from 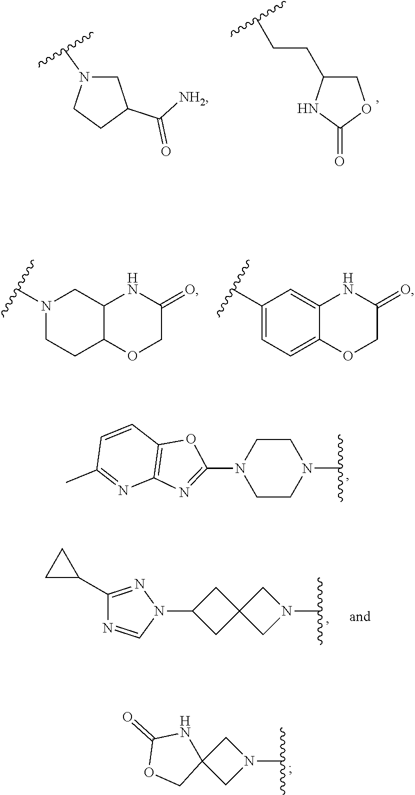
wherein a wavy line indicates the point of attachment to the carbonyl group of formula (I);
R² is selected from: 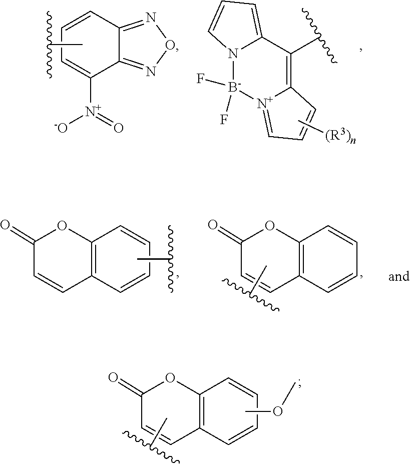
wherein a wavy line indicates the point of attachment to L;
R³ is selected from hydrogen, phenyl, and 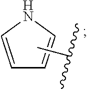
n is 1;
A is selected from:
(i) 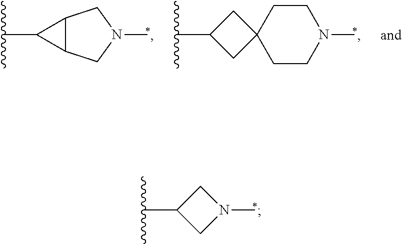
and
L is selected from a covalent bond, —O—, and —OCH₂—; and
(ii) 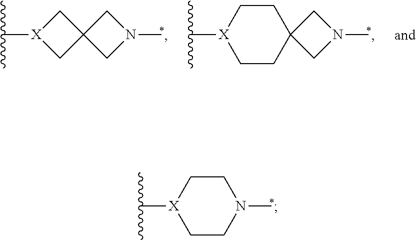
wherein
(i) X is N; and L is —CH₂—; or
(ii) X is CH; and L is selected from —CH₂—, —NH— and —O—; or
(iii) X and L, taken together, form a group 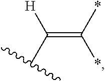
wherein the wavy line indicates the point of attachment of L to R², and the two asterisks indicate the points of attachment of X to the respective neighbouring atoms within ring A;
wherein a wavy line indicates the point of attachment to L; and
an asterisk indicates the point of attachment to the carbonyl group of formula is (I).

13. The compound of formula (I) according to claim 12, or a pharmaceutically acceptable salt thereof, wherein:
R¹ is selected from 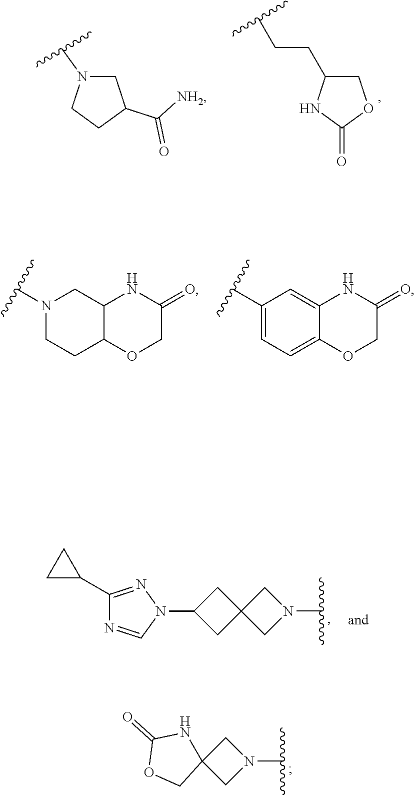
wherein a wavy line indicates the point of attachment to the carbonyl group of formula (I);
R² is selected from: 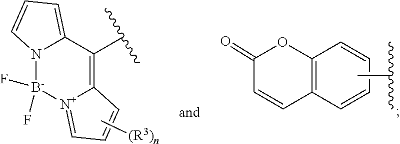
wherein a wavy line indicates the point of attachment to L;
R³ is selected from hydrogen and 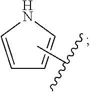
n is 1;
A is selected from:
(i) 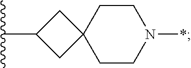
and
L is —O—; and
(ii) 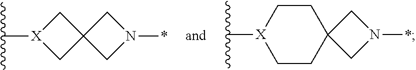
wherein
(i) X is CH; and L is selected from —CH₂— and —O—; or
(ii) X and L, taken together, form a group 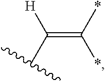
wherein the wavy line indicates the point of attachment of L to R², and the two asterisks indicate the points of attachment of X to the respective neighbouring atoms within ring A;
wherein a wavy line indicates the point of attachment to L; and
an asterisk indicates the point of attachment to the carbonyl group of formula (I).

14. The compound of formula (I) according to claim 13, or a pharmaceutically acceptable salt thereof, wherein:
R¹ is selected from 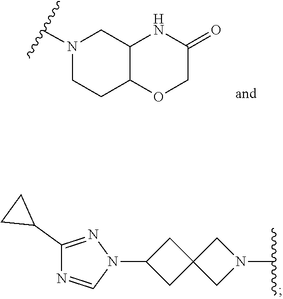 [SMILES: *N1CCC2OCC(=O)NC2C1 ; *N1CC2(CC(n3cnc(C4CC4)n3)C2)C1]
wherein a wavy line indicates the point of attachment to the carbonyl group of formula (I);
R² is: 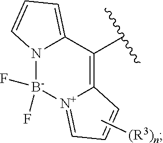
wherein a wavy line indicates the point of attachment to L;
R³ is hydrogen;
n is 1;
A is 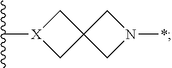
X is CH; and
L is —CH₂—;
wherein the wavy line indicates the point of attachment to L; and
the asterisk indicates the point of attachment to the carbonyl group of formula (I).

15. The compound of formula (I) according to claim 1, or a pharmaceutically acceptable salt thereof, wherein the compound of formula (I) is selected from:
(4aR,8aS)-6-(6-((4,4,5,5-tetramethyl-1,3,2-dioxaborolan-2-yl)methylene)-2-azaspiro[3.3]heptane-2-carbonyl)hexahydro-2H-pyrido[4,3-b][1,4]oxazin-3(4H)-one;
(4aR,8aS)-6-(3-(((2-oxo-2H-chromen-7-yl)oxy)methyl)azetidine-1-carbonyl)hexahydro-2H-pyrido[4,3-b][1,4]oxazin-3(4H)-one;
(4aR,8aS)-6-(3-(((2-oxo-2H-chromen-4-yl)oxy)methyl)azetidine-1-carbonyl)hexahydro-2H-pyrido[4,3-b][1,4]oxazin-3(4H)-one;
(4aR,8aS)-6-(6-((2-oxo-2H-chromen-4-yl)oxy)-2-azaspiro[3.3]heptane-2-carbonyl)hexahydro-2H-pyrido[4,3-b][1,4]oxazin-3(4H)-one;
(4aR,8aS)-6-(4-((7-methoxy-2-oxo-2H-chromen-4-yl)methyl)piperazine-1-carbonyl)hexahydro-2H-pyrido[4,3-b][1,4]oxazin-3(4H)-one;
(4aR,8aS)-6-(4-((7-nitrobenzo[c][1,2,5]oxadiazol-4-yl)amino)piperidine-1-carbonyl)hexahydro-2H-pyrido[4,3-b][1,4]oxazin-3(4H)-one;
(4aR,8aS)-6-(3-(((5,5-difluoro-5H-4λ⁴,5λ⁴-dipyrrolo[1,2-c:2′,1′-f][1,3,2]diazaborinin-10-yl)oxy)methyl)azetidine-1-carbonyl)hexahydro-2H-pyrido[4,3-b][1,4]oxazin-3(4H)-one;
(4aR,8aS)-6-(6-((5,5-difluoro-5H-4λ⁴,5λ⁴-dipyrrolo[1,2-c:2′,1′-f][1,3,2]diazaborinin-10-yl)oxy)-2-azaspiro[3.3]heptane-2-carbonyl)hexahydro-2H-pyrido[4,3-b][1,4]oxazin-3(4H)-one;
(4aR,8aS)-6-(4-((5,5-difluoro-5H-4λ⁴,5λ⁴-dipyrrolo[1,2-c:2′,1′-f][1,3,2]diazaborinin-10-yl)oxy)piperidine-1-carbonyl)hexahydro-2H-pyrido[4,3-b][1,4]oxazin-3(4H)-one;
(4aR,8aS)-6-(7-((5,5-difluoro-5H-4λ⁴,5λ⁴-dipyrrolo[1,2-c:2′,1′-f][1,3,2]diazaborinin-10-yl)oxy)-2-azaspiro[3.5]nonane-2-carbonyl)hexahydro-2H-pyrido[4,3-b][1,4]oxazin-3(4H)-one;
2-(6-((5,5-difluoro-5H-4λ⁴,5λ⁴-dipyrrolo[1,2-c:2′,1′-f][1,3,2]diazaborinin-10-yl)oxy)-2-azaspiro[3.3]heptane-2-carbonyl)-7-oxa-2,5-diazaspiro[3.4]octan-6-one;
(4aR,8aS)-6-(2-((5,5-difluoro-5H-4λ⁴,5λ⁴-dipyrrolo[1,2-c:2′,1′-f][1,3,2]diazaborinin-10-yl)oxy)-7-azaspiro[3.5]nonane-7-carbonyl)hexahydro-2H-pyrido[4,3-b][1,4]oxazin-3(4H)-one;
2-(2-((5,5-difluoro-5H-4λ4,5λ4-dipyrrolo[1,2-c:2′,1′-f][1,3,2]diazaborinin-10-yl)oxy)-7-azaspiro[3.5]nonane-7-carbonyl)-7-oxa-2,5-diazaspiro[3.4]octan-6-one;
(4aR,8aS)-6-((1R,5S,6R)-6-(((5,5-difluoro-5H-4λ⁴,5λ⁴-dipyrrolo[1,2-c:2′,1′-f][1,3,2]diazaborinin-10-yl)oxy)methyl)-3-azabicyclo[3.1.0]hexane-3-carbonyl)hexahydro-2H-pyrido[4,3-b][1,4]oxazin-3(4H)-one;
(4aR,8aS)-6-(6-((5,5-difluoro-5H-4λ⁴,5λ⁴-dipyrrolo[1,2-c:2′,1′-f][1,3,2]diazaborinin-10-yl)methylene)-2-azaspiro[3.3]heptane-2-carbonyl)hexahydro-2H-pyrido[4,3-b][1,4]oxazin-3(4H)-one;
(4aR,8aS)-6-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one;
(4aR,8aS)-6-(4-((5,5-difluoro-5H-4λ⁴,5λ⁴-dipyrrolo[1,2-c:2′,1′-f][1,3,2]diazaborinin-10-yl)methylene)piperidine-1-carbonyl)hexahydro-2H-pyrido[4,3-b][1,4]oxazin-3(4H)-one;
(4aR,8aS)-6-(7-((5,5-difluoro-5H-4λ⁴,5λ⁴-dipyrrolo[1,2-c:2′,1′-f][1,3,2]diazaborinin-10-yl)methylene)-2-azaspiro[3.5]nonane-2-carbonyl)hexahydro-2H-pyrido[4,3-b][1,4]oxazin-3(4H)-one;
(4aR,8aS)-6-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonane-2-carbonyl)hexahydro-2H-pyrido[4,3-b][1,4]oxazin-3(4H)-one;
2-(7-((5,5-difluoro-5H-4λ⁴,5λ⁴-dipyrrolo[1,2-c:2′,1′-f][1,3,2]diazaborinin-10-yl)methylene)-2-azaspiro[3.5]nonane-2-carbonyl)-7-oxa-2,5-diazaspiro[3.4]octan-6-one;
2-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonane-2-carbonyl)-7-oxa-2,5-diazaspiro[3.4]octan-6-one;
2-(6-((5,5-difluoro-5H-4λ⁴,5λ⁴-dipyrrolo[1,2-c:2′,1′-f][1,3,2]diazaborinin-10-yl)methylene)-2-azaspiro[3.3]heptane-2-carbonyl)-7-oxa-2,5-diazaspiro[3.4]octan-6-one;
2-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)-7-oxa-2,5-diazaspiro[3.4]octan-6-one;
6-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)-2H-benzo[b][1,4]oxazin-3(4H)-one;
(6-(3-cyclopropyl-1H-1,2,4-triazol-1-yl)-2-azaspiro[3.3]heptan-2-yl)(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)methanone;
(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)(4-(5-methyloxazolo[4,5-b]pyridin-2-yl)piperazin-1-yl)methanone;
(S)-1-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)pyrrolidine-3-carboxamide;
(R)-4-(3-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)-3-oxopropyl)oxazolidin-2-one;
(4aR,8aS)-6-(6-((5,5-difluoro-3-phenyl-5H-4λ⁴,5)⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one;
(4aR,8aS)-6-(6-((5,5-difluoro-3-(1H-pyrrol-2-yl)-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one;
(S)-(3-(1H-1,2,3-triazol-5-yl)pyrrolidin-1-yl)(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)methanone;
(S)-(3-(1H-1,2,3-triazol-5-yl)pyrrolidin-1-yl)(7-((5,5-difluoro-5H-4λ⁴,5λ⁴-dipyrrolo[1,2-c:2′,1′-f][1,3,2]diazaborinin-10-yl)methyl)-2-azaspiro[3.5]nonan-2-yl)methanone;
(6-(3-cyclopropyl-1H-1,2,4-triazol-1-yl)-2-azaspiro[3.3]heptan-2-yl)(7-((5,5-difluoro-5H-4λ⁴,5λ⁴-dipyrrolo[1,2-c:2′,1′-f][1,3,2]diazaborinin-10-yl)methyl)-2-azaspiro[3.5]nonan-2-yl)methanone;
6-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonane-2-carbonyl)-2H-benzo[b][1,4]oxazin-3(4H)-one;
(R)-4-(3-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonan-2-yl)-3-oxopropyl)oxazolidin-2-one;
(S)-1-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonane-2-carbonyl)pyrrolidine-3-carboxamide;
(7-((5,5-difluoro-5H-4λ⁴,5λ⁴-dipyrrolo[1,2-c:2′,1′-f][1,3,2]diazaborinin-10-yl)methyl)-2-azaspiro[3.5]nonan-2-yl)(4-(5-methyloxazolo[4,5-b]pyridin-2-yl)piperazin-1-yl)methanone;
(4aR,8aS)-6-(2-((5,5-difluoro-5H-4λ⁴,5λ⁴-dipyrrolo[1,2-c:2′,1′-f][1,3,2]diazaborinin-10-yl)methyl)-7-azaspiro[3.5]nonane-7-carbonyl)hexahydro-2H-pyrido[4,3-b][1,4]oxazin-3(4H)-one;
2-(2-((5,5-difluoro-5H-4λ⁴,5λ⁴-dipyrrolo[1,2-c:2′,1′-f][1,3,2]diazaborinin-10-yl)methyl)-7-azaspiro[3.5]nonane-7-carbonyl)-7-oxa-2,5-diazaspiro[3.4]octan-6-one;
(S)-(3-(1H-1,2,3-triazol-5-yl)pyrrolidin-1-yl)(2-((5,5-difluoro-5H-4λ⁴,5λ4-dipyrrolo[1,2-c:2′,1′-f][1,3,2]diazaborinin-10-yl)methyl)-7-azaspiro[3.5]nonan-7-yl)methanone;
(6-(3-cyclopropyl-1H-1,2,4-triazol-1-yl)-2-azaspiro[3.3]heptan-2-yl)(2-((5,5-difluoro-5H-4λ⁴,5λ⁴-dipyrrolo[1,2-c:2′,1′-f][1,3,2]diazaborinin-10-yl)methyl)-7-azaspiro[3.5]nonan-7-yl)methanone;
6-(2-((5,5-difluoro-5H-4λ⁴,5λ⁴-dipyrrolo[1,2-c:2′,1′-f][1,3,2]diazaborinin-10-yl)methyl)-7-azaspiro[3.5]nonane-7-carbonyl)-2H-benzo[b][1,4]oxazin-3(4H)-one;
(R)-4-(3-(2-((5,5-difluoro-5H-4λ⁴,5λ⁴-dipyrrolo[1,2-c:2′,1′-f][1,3,2]diazaborinin-10-yl)methyl)-7-azaspiro[3.5]nonan-7-yl)-3-oxopropyl)oxazolidin-2-one;
(S)-1-(2-((5,5-difluoro-5H-4λ⁴,5λ⁴-dipyrrolo[1,2-c:2′,1′-f][1,3,2]diazaborinin-10-yl)methyl)-7-azaspiro[3.5]nonane-7-carbonyl)pyrrolidine-3-carboxamide;
(2-((5,5-difluoro-5H-4λ⁴,5λ⁴-dipyrrolo[1,2-c:2′,1′-f][1,3,2]diazaborinin-10-yl)methyl)-7-azaspiro[3.5]nonan-7-yl)(4-(5-methyloxazolo[4,5-b]pyridin-2-yl)piperazin-1-yl)methanone; and
(6-(3-cyclopropyl-1H-1,2,4-triazol-1-yl)-2-azaspiro[3.3]heptan-2-yl)(6-((5,5-difluoro-3-(1H-pyrrol-2-yl)-5H-5λ⁴,6λ⁴-dipyrrolo[1,2-c:2′,1′-f][1,3,2]diazaborinin-10-yl)methyl)-2-azaspiro[3.3]heptan-2-yl)methanone.

16. The compound of formula (I) according to claim 15, or a pharmaceutically acceptable salt thereof, wherein the compound of formula (I) is selected from:
(4aR,8aS)-6-(6-((4,4,5,5-tetramethyl-1,3,2-dioxaborolan-2-yl)methylene)-2-azaspiro[3.3]heptane-2-carbonyl)hexahydro-2H-pyrido[4,3-b][1,4]oxazin-3(4H)-one;
(4aR,8aS)-6-(7-((5,5-difluoro-5H-4λ⁴,5λ⁴-dipyrrolo[1,2-c:2′,1′-f][1,3,2]diazaborinin-10-yl)oxy)-2-azaspiro[3.5]nonane-2-carbonyl)hexahydro-2H-pyrido[4,3-b][1,4]oxazin-3(4H)-one;
2-(6-((5,5-difluoro-5H-4λ⁴,5λ⁴-dipyrrolo[1,2-c:2′,1′-f][1,3,2]diazaborinin-10-yl)oxy)-2-azaspiro[3.3]heptane-2-carbonyl)-7-oxa-2,5-diazaspiro[3.4]octan-6-one;
2-(2-((5,5-difluoro-5H-4λ⁴,5λ⁴-dipyrrolo[1,2-c:2′,1′-f][1,3,2]diazaborinin-10-yl)oxy)-7-azaspiro[3.5]nonane-7-carbonyl)-7-oxa-2,5-diazaspiro[3.4]octan-6-one;
(4aR,8aS)-6-(6-((5,5-difluoro-5H-4λ⁴,5λ⁴-dipyrrolo[1,2-c:2′,1′-f][1,3,2]diazaborinin-10-yl)methylene)-2-azaspiro[3.3]heptane-2-carbonyl)hexahydro-2H-pyrido[4,3-b][1,4]oxazin-3(4H)-one;
(4aR,8aS)-6-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one;
(4aR,8aS)-6-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonane-2-carbonyl)hexahydro-2H-pyrido[4,3-b][1,4]oxazin-3(4H)-one;
2-(7-((5,5-difluoro-5H-4λ⁴,5λ⁴-dipyrrolo[1,2-c:2′,1′-f][1,3,2]diazaborinin-10-yl)methyl)-2-azaspiro[3.5]nonane-2-carbonyl)-7-oxa-2,5-diazaspiro[3.4]octan-6-one;
2-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)-7-oxa-2,5-diazaspiro[3.4]octan-6-one;
6-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)-2H-benzo[b][1,4]oxazin-3(4H)-one;
(6-(3-cyclopropyl-1H-1,2,4-triazol-1-yl)-2-azaspiro[3.3]heptan-2-yl)(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)methanone;
(S)-1-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)pyrrolidine-3-carboxamide;
(R)-4-(3-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)-3-oxopropyl)oxazolidin-2-one;
(4aR,8aS)-6-(6-((5,5-difluoro-3-phenyl-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one; and
(4aR,8aS)-6-(6-((5,5-difluoro-3-(1H-pyrrol-2-yl)-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one.

17. The compound of formula (I) according to claim 16, or a pharmaceutically acceptable salt thereof, wherein the compound of formula (I) is selected from:
(4aR,8aS)-6-(6-((5,5-difluoro-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one;
(6-(3-cyclopropyl-1H-1,2,4-triazol-1-yl)-2-azaspiro[3.3]heptan-2-yl)(6-((5,5-difluoro-5H-4λ⁴,5λ⁴-dipyrrolo[1,2-c:2′,1′-f][1,3,2]diazaborinin-10-yl)methyl)-2-azaspiro[3.3]heptan-2-yl)methanone; and
(4aR,8aS)-6-(6-((5,5-difluoro-3-(1H-pyrrol-2-yl)-5H-4λ⁴,5λ⁴-dipyrrolo[1,2-c:2′,1′-f][1,3,2]diazaborinin-10-yl)methyl)-2-azaspiro[3.3]heptane-2-carbonyl)hexahydro-2H-pyrido[4,3-b][1,4]oxazin-3(4H)-one.

18. A process of manufacturing the compounds of formula (I) according to any one of claims 1 to 17, comprising:
(a) reacting a first amine 1, 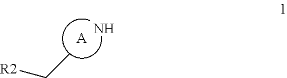
wherein A and R² are as defined in any one of claims 1 to 17;
with a second amine selected from: 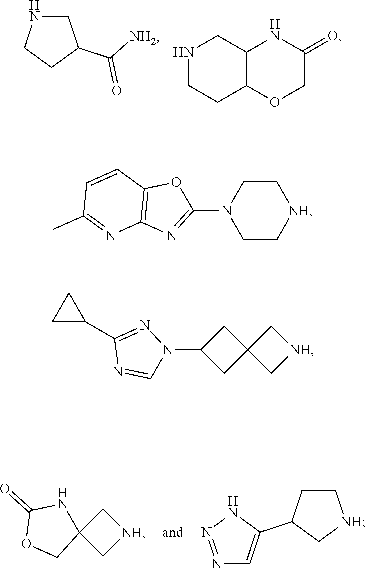 [SMILES: NC(=O)C1CCNC1 ; O=C1COC2CCNCC2N1 ; Cc1ccc2oc(N3CCNCC3)nc2n1 ; c1nc(C2CC2)nn1C1CC2(CNC2)C1 ; O=C1NC2(CNC2)CO1 ; c1nn[nH]c1C1CCNC1]
in the presence of a base and a urea forming reagent,
to form said compound of formula (I); or
(b) reacting an amine 1, 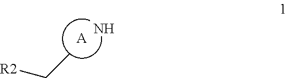
wherein A and R² are as defined in any one of claims 1 to 17;
with a carboxylic acid selected from: 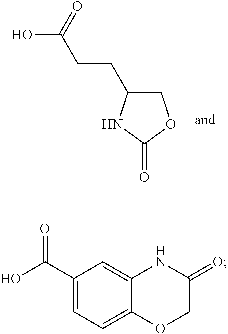 [SMILES: O=C(O)CCC1COC(=O)N1 ; O=C1COc2ccc(C(=O)O)cc2N1]
in the presence of a base and an amide coupling reagent,
to form said compound of formula (I).

19. A compound of formula (I) according to any one of claims 1 to 17, when manufactured according to the process of claim 18.

20. A compound of formula (I) according to any one of claims 1 to 17, for use in monoacylglycerol lipase (MAGL) occupancy studies.

21. A compound of formula (I) according to any one of claims 1 to 17, for use in diagnostic imaging of monoacylglycerol lipase (MAGL) in a mammal.

22. A compound of formula (I) according to any one of claims 1 to 17, for use in generating monoacylglycerol lipase (MAGL) equilibrium and kinetic binding data.

23. Use of a compound of formula (I) according to any one of claims 1 to 17 in monoacylglycerol lipase (MAGL) occupancy studies.

24. Use of a compound of formula (I) according to any one of claims 1 to 17 in diagnostic imaging of monoacylglycerol lipase (MAGL) in a mammal.

25. Use of a compound of formula (I) according to any one of claims 1 to 17 for generating monoacylglycerol lipase (MAGL) equilibrium and kinetic binding data.

26. A method of studying monoacylglycerol lipase (MAGL) occupancy, comprising contacting MAGL with a compound of formula (I) according to any one of claims 1 to 17.

27. A method of diagnostic imaging of monoacylglycerol lipase (MAGL) in a mammal, comprising contacting MAGL with a compound of formula (I) according to any one of claims 1 to 17.

28. A method of generating monoacylglycerol lipase (MAGL) equilibrium and kinetic binding data, comprising contacting MAGL with a compound of formula (I) according to any one of claims 1 to 17.

29. The invention as described hereinbefore.
